(19) United States
(12) Patent Application Publication
(10) Pub. No.: US 2010/0197687 A1
(43) Pub. Date: Aug. 5, 2010

(54) Indoles Useful in the Treatment of Inflammation

(76) Inventors: Benjamin Pelcman, Solna (SE); Kristofer Olofsson, Solna (SE); Martins Katkevics, Riga (LV); Vita Ozola, Riga (LV); Edgars Suna, Riga (LV); Ivars Kalvins, Riga (LV); Peteris Trapencieris, Riga (LV); Dace Katkevica, Riga (LV); Wesley Schaal, Solna (SE)

Correspondence Address: MORGAN LEWIS & BOCKIUS LLP, 1111 PENNSYLVANIA AVENUE NW, WASHINGTON, DC, 20004, US

(21) Appl. No.: 11/795,632

(22) PCT Filed: Dec. 22, 2005
(86) PCT No.: PCT/GB2005/004982

§ 371 Date: Mar. 30, 2009

Related U.S. Application Data
(60) Provisional application No. 60/644,525, filed on Jan. 19, 2005.
(60) Provisional application No. 60/644,520, filed on Jan. 19, 2005.

(51) Int. Cl. A61K 31/5355; C07D 209/12; A61K 31/404; C07D 413/04; C07D 403/06; A61K 31/496; C07D 401/04; A61K 31/4439; A61P 29/00

(52) U.S. Cl. 514/235.2; 548/492; 514/419; 544/143; 544/373; 514/254.09; 546/278.1; 514/339

(57) ABSTRACT

There is provided compounds of formula I,

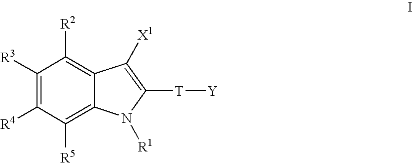

wherein T, Y, X¹, R¹, R², R³, R⁴ and R⁵ have meanings given in the description, and pharmaceutically-acceptable salts thereof, which compounds are useful in the treatment of diseases in which inhibition of the activity of a member of the MAPEG family is desired and/or required, and particularly in the treatment of inflammation.

### FIELD OF THE INVENTION

This invention relates to novel pharmaceutically-useful compounds, which compounds are useful as inhibitors of enzymes belonging to the membrane-associated proteins in the eicosanoid and glutathione metabolism (MAPEG) family. Members of the MAPEG family include the microsomal prostaglandin E synthase-1 (mPGES-1), 5-lipoxygenase-activating protein (FLAP), leukotriene C₄ synthase and microsomal glutathione S-transferases (MGST1, MGST2 and MGST3). The compounds are of potential utility in the treatment of inflammatory diseases including respiratory diseases. The invention also relates to the use of such compounds as medicaments, to pharmaceutical compositions containing them, and to synthetic routes for their production.

### BACKGROUND OF THE INVENTION

There are many diseases/disorders that are inflammatory in their nature. One of the major problems associated with existing treatments of inflammatory conditions is a lack of efficacy and/or the prevalence of side effects (real or perceived).

Inflammatory diseases that affect the population include asthma, inflammatory bowel disease, rheumatoid arthritis, osteoarthritis, rhinitis, conjunctivitis and dermatitis.

Inflammation is also a common cause of pain. Inflammatory pain may arise for numerous reasons, such as infection, surgery or other trauma. Moreover, several diseases including malignancies and cardiovascular diseases are known to have inflammatory components adding to the symptomatology of the patients.

Asthma is a disease of the airways that contains elements of both inflammation and bronchoconstriction. Treatment regimens for asthma are based on the severity of the condition. Mild cases are either untreated or are only treated with inhaled β-agonists which affect the bronchoconstriction element, whereas patients with more severe asthma typically are treated regularly with inhaled corticosteroids which to a large extent are anti-inflammatory in their nature.

Another common disease of the airways with inflammatory and bronchoconstrictive components is chronic obstructive pulmonary disease (COPD). The disease is potentially lethal, and the morbidity and mortality from the condition is considerable. At present, there is no known pharmacological treatment capable of changing the course of the disease.

The cyclooxygenase (COX) enzyme exists in two forms, one that is constitutively expressed in many cells and tissues (COX-1), and one that is induced by pro-inflammatory stimuli, such as cytokines, during an inflammatory response (COX-2).

COXs metabolise arachidonic acid to the unstable intermediate prostaglandin H₂ (PGH₂). PGH₂ is further metabolized to other prostaglandins including PGE₂, PGF_{2α}, PGD₂, prostacyclin and thromboxane A₂. These arachidonic acid metabolites are known to have pronounced physiological and pathophysiological activity including pro-inflammatory effects.

PGE₂ in particular is known to be a strong pro-inflammatory mediator, and is also known to induce fever and pain. Consequently, numerous drugs have been developed with a view to inhibiting the formation of PGE₂, including “NSAIDs” (non-steroidal antiinflammatory drugs) and “coxibs” (selective COX-2 inhibitors). These drugs act predominantly by inhibition of COX-1 and/or COX-2, thereby reducing the formation of PGE₂.

However, the inhibition of COXs has the disadvantage that it results in the reduction of the formation of all metabolites of arachidonic acid, some of which are known to have beneficial properties. In view of this, drugs which act by inhibition of COXs are therefore known/suspected to cause adverse biological effects. For example, the non-selective inhibition of COXs by NSAIDs may give rise to gastrointestinal side-effects and affect platelet and renal function. Even the selective inhibition of COX-2 by coxibs, whilst reducing such gastrointestinal side-effects, is believed to give rise to cardiovascular problems.

An alternative treatment of inflammatory diseases that does not give rise to the above-mentioned side effects would thus be of real benefit in the clinic. In particular, a drug that inhibits (preferably selectively) the transformation of PGH₂ to the pro-inflammatory mediator PGE₂ might be expected to reduce the inflammatory response in the absence of a corresponding reduction of the formation of other, beneficial arachidonic acid metabolites. Such inhibition would accordingly be expected to alleviate the undesirable side-effects mentioned above.

PGH₂ may be transformed to PGE₂ by prostaglandin E synthases (PGES). Two microsomal prostaglandin E synthases (mPGES-1 and mPGES-2), and one cytosolic prostaglandin E synthase (cPGES) have been described.

The leukotrienes (LTs) are formed from arachidonic acid by a set of enzymes distinct from those in the COX/PGES pathway. Leukotriene B4 is known to be a strong proinflammatory mediator, while the cysteinyl-containing leukotrienes C₄, D₄ and E₄ (CysLTs) are mainly very potent bronchoconstrictors and have thus been implicated in the pathobiology of asthma. The biological activities of the CysLTs are mediated through two receptors designated CysLT₁ and CysLT₂. As an alternative to steroids, leukotriene receptor antagonists (LTRas) have been developed in the treatment of asthma. These drugs may be given orally, but do not control inflammation satisfactorily. The presently used LTRas are highly selective for CysLT₁. It may be hypothesised that better control of asthma, and possibly also COPD, may be attained if the activity of both of the CysLT receptors could be reduced. This may be achieved by developing unselective LTRas, but also by inhibiting the activity of proteins, e.g. enzymes, involved in the synthesis of the CysLTs. Among these proteins, 5-lipoxygenase, 5-lipoxygenase-activating protein (FLAP), and leukotriene C₄ synthase may be mentioned. A FLAP inhibitor would also decrease the formation of the proinflammatory LTB₄.

mPGES-1, FLAP and leukotriene C₄ synthase belong to the membrane-associated proteins in the eicosanoid and glutathione metabolism. (MAPEG) family. Other members of this family include the microsomal glutathione S-transferases (MGST1, MGST2 and MGST3). For a review, c.f. P.-J. Jacobsson et al in Am. J. Respir. Crit. Care Med. 161, S20 (2000). It is well known that compounds prepared as antagonists to one of the MAPEGs may also exhibit inhibitory activity towards other family members, c.f. J. H Hutchinson et al in J. Med. Chem. 38, 4538 (1995) and D. Claveau et al in J. Immunol. 170, 4738 (2003). The former paper also describes that such compounds may also display notable cross-reactivity with proteins in the arachidonic acid cascade that do not belong to the MAPEG family, e.g. 5-lipoxygenase.

Thus, agents that are capable of inhibiting the action of mPGES-1, and thus reducing the formation of the specific arachidonic acid metabolite PGE₂, are likely to be of benefit in the treatment of inflammation. Further, agents that are capable of inhibiting the action of the proteins involved in the synthesis of the leukotrienes are also likely to be of benefit in the treatment of asthma and COPD.

### PRIOR ART

Certain specific 1(N)-phenylindole-2-carboxylate derivatives have been disclosed by Rajur et al in Ind. J. Chem Section B: Organic Chemistry Including Medicinal Chemistry, 31B, 551 (1992) as chemical intermediates useful in the synthesis of antiallergic agents.

Indole-based compounds have been disclosed in international patent applications WO 96/03377, WO 01/00197, WO 03/044014 and WO 03/057670, U.S. Pat. Nos. 5,189,054, 5,294,722 and 4,960,786 and European patent applications EP 429 257, EP 483 881, EP 547 556, EP 639 573 and EP 1 314 733. In particular European patent application EP 488 532 and U.S. Pat. Nos. 5,236,916 and 5,374,615 disclose 1(N)-phenylindole-2-carboxylates as antihypertensive agents and as chemical intermediates. However, none of these documents disclose or suggest the use of such compounds in the treatment of inflammation.

Indoles have also been disclosed for potential use in the treatment of inflammation in international patent applications WO 99/43672, WO 98/08818, WO 99/43654, WO 99/43651, WO 99/05104 and WO 03/029212, European patent application EP 986 666 and U.S. Pat. Nos. 6,500,853 and 6,630,496. However, there is no specific disclosure in any of these documents of indole-2-carboxylates in which an aromatic group is directly attached via the indole nitrogen.

International patent application WO 01/30343, and European patent application EP 186 367, also mention indoles for potential use as PPAR-É binding agents, and in the treatment of inflammation, respectively. However, these documents do not mention or suggest compounds in which the benzenoid moiety of the indole is substituted (directly or via a linking group) with an aromatic ring. Further, Dropinski et al, Bioorganic and Medicinal Chemistry Letters, 15 (2005) 5035-5038 discloses various indoles for use as PPAR-É partial agonists. There is no mention or suggestion of the use of such compounds as inhibitors of mPGES.

Various 1(N)-benzylindole-2-carboxylates and derivatives thereof are known from international patent applications WO 99/33800 as Factor Xa inhibitors; WO 99/07678, WO 99/07351, WO 00/46198, WO 00/46197, WO 00/46195 and WO 00/46199 as inhibitors of MCP-1; international patent application WO 96/18393 as inhibitors of IL-8; international patent applications WO 93/25546 and WO 94/13662, European patent application EP 535 924 A1 and U.S. Pat. No. 5,081,138 as inhibitors of leukotriene biosynthesis; international patent application WO 02/30895 as PPAR-É binding agents; and European patent application EP 166 591 as prostaglandin antagonists. Further, unpublished international patent application PCT/GB2004/002996 discloses such compounds for use as inhibitors of mPGES and thus in the treatment of inflammation. However, there is no specific disclosure in any of these documents of indole-2-carboxylates in which an aromatic group is directly attached via the indole nitrogen.

Further, unpublished international patent applications PCT/GB2005/002404, PCT/GB2005/002391 and PCT/GB2005/002396 disclose indoles for use as inhibitors of mPGES and thus in the treatment of inflammation. However, these documents only disclose indoles that are substituted at the 3-position with either H, halo, an aromatic group or an amino group (or derivative thereof), and which indoles are directly substituted at the benzenoid moiety with an aromatic group.

Finally, international patent application WO 94/14434 discloses structurally similar indoles as endothelin receptor antagonists. There is no specific disclosure in this document of compounds with indole-2-carboxylates in which an aromatic group is directly attached via the indole nitrogen, nor of compounds in which aromatic and heteroaromatic moieties are attached to the benzenoid part of the indole via a linking group.

### DISCLOSURE OF THE INVENTION

According to a first aspect of the invention there is provided a compound of formula I,

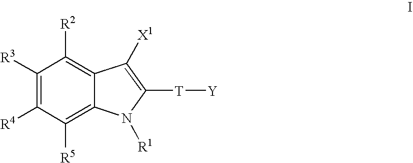

wherein
one of the groups R², R³, R⁴ and R⁵ represents -D-E and:
a) the other groups are independently selected from hydrogen, G¹, an aryl group, a heteroaryl group (which latter two groups are optionally substituted by one or more substituents selected from A), C₁₋₈ alkyl and a heterocycloalkyl group (which latter two groups are optionally substituted by one or more substituents selected from G¹ and/or Z¹); and/or
b) any two other groups which are adjacent to each other are optionally linked to form, along with two atoms of the essential benzene ring in the compound of formula I, a 3- to 8-membered ring, optionally containing 1 to 3 heteroatoms, which ring is itself optionally substituted by one or more substituents selected from halo, —R⁶, —OR⁶ and ═O;
D represents a single bond, —O—, —C(R⁷)(R⁸)—, C₂₋₄ alkylene, —C(O)— or —S(O)ₘ—;
R¹ and E independently represent an aryl group or a heteroaryl group, both of which groups are optionally substituted by one or more substituents selected from A;
R⁷ and R⁸ independently represent H, halo or C₁₋₆ alkyl, which latter group is optionally substituted by halo, or R⁷ and R⁸ may together form, along with the carbon atom to which they are attached, a 3- to 6-membered ring, which ring optionally contains a heteroatom and is optionally substituted by one or more substituents selected from halo and C₁₋₃ alkyl, which latter group is optionally substituted by one or more halo substituents;
X¹ represents H, halo, —N(R^{9a})-J-R^{10a} or -Q-X²;
J represents a single bond, —C(O)— or —S(O)ₘ—;
Q represents a single bond, —O—, —C(O)— or —S(O)ₘ—;
X² represents:
(a) an aryl group or a heteroaryl group, both of which are optionally substituted by one or more substituents selected from A; or
(b) C₁₋₈ alkyl or a heterocycloalkyl group, both of which are optionally substituted by one or more substituents selected from G¹ and/or Z¹; or, when Q is a single bond,
(c) cyano;
T represents:
(a) a single bond;
(b) a C₁₋₈ alkylene or a C₂₋₈ heteroalkylene chain, both of which latter two groups:
(i) optionally contain one or more unsaturations (for example double or triple bonds);
(ii) are optionally substituted by one or more substituents selected from G¹ and/or Z¹; and/or
(iii) may comprise an additional 3- to 8-membered ring formed between any one or more (e.g. one or two) members of the C₁₋₈ alkylene or C₂₋₈ heteroalkylene chain, which ring optionally contains 1 to 3 heteroatoms and/or 1 to 3 unsaturations (for example double or triple bonds) and which ring is itself optionally substituted by one or more substituents selected from G¹ and/or Z¹;
   (c) an arylene group or a heteroarylene group, both of which groups are optionally substituted by one or more substituents selected from A; or

### (d) -T¹-W¹-T²-;

one of T¹ and T² represents a C₁₋₈ alkylene or a C₂₋₈ heteroalkylene chain, both of which latter two groups:
(i) optionally contain one or more unsaturations (for example double or triple bonds);
(ii) are optionally substituted by one or more substituents selected from G¹ and/or Z¹; and/or
(iii) may comprise an additional 3- to 8-membered ring formed between any one or more (e.g. one or two) members of the C₁₋₈ alkylene or C₂₋₈ heteroalkylene chain, which ring optionally contains 1 to 3 heteroatoms and/or 1 to 3 unsaturations (for example double or triple bonds) and which ring is itself optionally substituted by one or more substituents selected from G¹ and/or Z¹;
   and the other represents an arylene group or a heteroarylene group chain, both of which groups are optionally substituted by one or more substituents selected from A;
   W¹ represents —O— or —S(O)ₘ—;
   m represents, on each occasion when mentioned above, 0, 1 or 2;
   Y represents —C(H)(CF₃)OH, —C(O)CF₃, —C(OH)₂CF₃, —C(O)OR^{9b}, —S(O)₃R^{9c}, —P(O)(OR^{9d})₂, —P(O)(OR^{9e})N(R^{10f})R^{9f}, —P(O)(N(R^{10g})R^{9g})₂, —B(OR^{9h})₂, —C(CF₃)₂OH, —S(O)₂N(R¹⁰ⁱ)R⁹ⁱ or any one of the following groups:

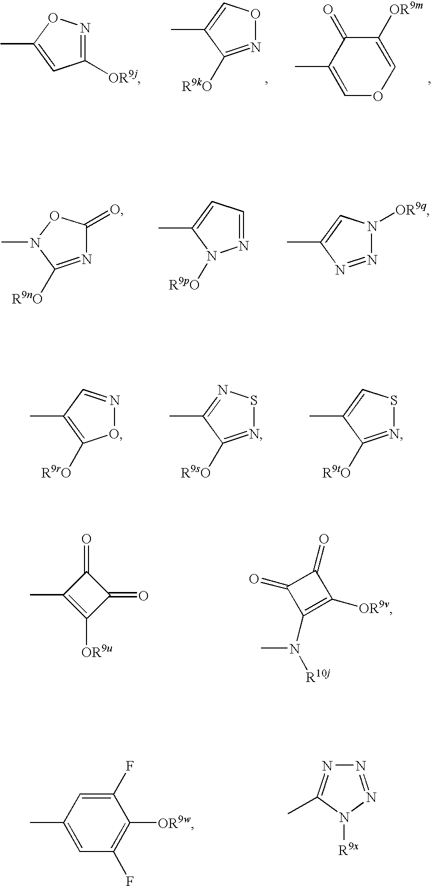

R⁶, R^{9a} to R^{9x}, R^{10a}, R^{10f}, R^{10g}, R¹⁰ⁱ and R^{10j} independently represent, on each occasion when mentioned above:
I) hydrogen;
II) an aryl group or a heteroaryl group, both of which are optionally substituted by one or more substituents selected from B; or
III) C₁₋₈ alkyl or a heterocycloalkyl group, both of which are optionally substituted by one or more substituents selected from G¹ and/or Z¹; or
any pair of R^{9a} to R^{9x} and R^{10a}, R^{10f}, R^{10g}, R¹⁰ⁱ or R^{10j}, may be linked together to form, along with the atom(s) and/or group(s) to which they are attached, a 3- to 8-membered ring, optionally containing 1 to 3 heteroatoms and/or 1 to 3 double bonds, which ring is optionally substituted by one or more substituents selected from G¹ and/or Z¹;
A represents, on each occasion when mentioned above:
I) an aryl group or a heteroaryl group, both of which are optionally substituted by one or more substituents selected from B;
II) C₁₋₈ alkyl or a heterocycloalkyl group, both of which are optionally substituted by one or more substituents selected from G¹ and/or Z¹; or
III) a G¹ group;
G¹ represents, on each occasion when mentioned above, halo, cyano, —N₃, —NO₂, —ONO₂ or -A¹-R^{11a};
wherein A¹ represents a single bond or a spacer group selected from —C(O)A²-, —S(O)₂A³-, —N(R^{12a})A⁴- or —OA⁵-, in which:
A² represents a single bond, —O—, —N(R^{12b})— or —C(O)—;
A³ represents a single bond, —O— or —N(R^{12c})—;
A⁴ and A⁵ independently represent a single bond, —C(O)—, —C(O)N(R^{12d})—, —C(O)O—, —S(O)₂— or —S(O)₂N(R^{12e})—;
Z¹ represents, on each occasion when mentioned above, ═O, ═S, ═NOR^{11b}, ═NS(O)₂N(R^{12f})R^{11c}, ═NCN or ═C(H)NO₂;
B represents, on each occasion when mentioned above:
I) an aryl group or a heteroaryl group, both of which are optionally substituted by one or more substituents selected from G²;
II) C₁₋₈ alkyl or a heterocycloalkyl group, both of which are optionally substituted by one or more substituents selected from G² and/or Z²; or
III) a G² group;
G² represents, on each occasion when mentioned above, halo, cyano, —N₃, —NO₂, —ONO₂ or -A⁶-R^{13a};
wherein A⁶ represents a single bond or a spacer group selected from —C(O)A⁷-, —S(O)₂A⁸-, —N(R^{14a})A⁹- or —OA¹⁰-, in which:
A⁷ represents a single bond, —O—, —N(R^{14b})— or —C(O)—;
A⁸ represents a single bond, —O— or —N(R^{14c})—;
A⁹ and A¹⁰ independently represent a single bond, —C(O)—, —C(O)N(R^{14d})—, —C(O)O—, —S(O)₂— or —S(O)₂N(R^{14e})—;
Z² represents, on each occasion when mentioned above, ═O, ═S, ═NOR^{13b}, ═NS(O)₂N(R^{14f})R^{13c}, ═NCN or ═C(H)NO₂;
R^{11a}, R^{11b}, R^{11c}, R^{12a}, R^{12b}, R^{12c}, R^{12d}, R^{12e}, R^{12f}, R^{13a}, R^{13b}, R^{13c}, R^{14a}, R^{14b}, R^{14c}, R^{14d}, R^{14e} and R^{14f} are independently selected from:
i) hydrogen;
ii) an aryl group or a heteroaryl group, both of which are optionally substituted by one or more substituents selected from G³;
iii) C₁₋₈ alkyl or a heterocycloalkyl group, both of which are optionally substituted by G³ and/or Z³; or
any pair of R^{11a} to R^{11c} and R^{12a} to R^{12f}, and/or R^{13a} to R^{13c} and R^{14a} to R^{14f}, may, for example when present on the same or on adjacent atoms, be linked together to form with those, or other relevant, atoms a further 3- to 8-membered ring, optionally containing 1 to 3 heteroatoms and/or 1 to 3 double bonds, which ring is optionally substituted by one or more substituents selected from G³ and/or Z³;
G³ represents, on each occasion when mentioned above, halo, cyano, —N₃, —NO₂, —ONO₂ or -A¹¹-R^{15a};
wherein A¹¹ represents a single bond or a spacer group selected from —C(O)A^{l2}-, —S(O)₂A¹³-, —N(R^{16a})A¹⁴- or —OA¹⁵-, in which:
A^{l2} represents a single bond, —O—, —N(R^{16b})— or —C(O)—;
A¹³ represents a single bond, —O— or —N(R^{16c})—;
A¹⁴ and A¹⁵ independently represent a single bond, —C(O)—, —C(O)N(R^{16d})—, —C(O)O—, —S(O)₂— or —S(O)₂N(R^{16e})—;
Z³ represents, on each occasion when mentioned above, ═O, ═S, ═NOR^{15b}, ═NS(O)₂N(R^{16f})R^{15c}, ═NCN or ═C(H)NO₂;
R^{15a}, R^{15b}, R^{15c}, R^{16a}, R^{16b}, R^{16c}, R^{16d}, R^{16e} and R^{16f} are independently selected from:
i) hydrogen;
ii) C₁₋₆ alkyl or a heterocycloalkyl group, both of which groups are optionally substituted by one or more substituents selected from halo, C₁₋₄ alkyl, —N(R^{17a})R^{18a}, —OR^{17b} and ═O; and
iii) an aryl or heteroaryl group, both of which are optionally substituted by one or more substituents selected from halo, C₁₋₄ alkyl, —N(R^{17c})R^{18b} and —OR^{17d}; or
any pair of R^{15a} to R^{15c} and R^{16a} to R^{16f} may, for example when present on the same or on adjacent atoms, be linked together to form with those, or other relevant, atoms a further 3- to 8-membered ring, optionally containing 1 to 3 heteroatoms and/or 1 to 3 double bonds, which ring is optionally substituted by one or more substituents selected from halo, C₁₋₄ alkyl, —N(R^{17e})R^{18c}, —OR^{17f} and ═O;
R^{17a}, R^{17b}, R^{17c}, R^{17d}, R^{17e}, R^{17f}, R^{18a}, R^{18b} and R^{18c} are independently selected from hydrogen and C₁₋₄ alkyl, which latter group is optionally substituted by one or more halo groups;
wherein:
(I) when X¹ represents H, halo, —N(R^{9a})-J-R^{10a} or -Q-X² in which Q is a single bond and X² is an aryl or heteroaryl group (both of which are optionally substituted by one or more substituents selected from A), then T does not represent a single bond when Y is —C(O)OR^{9b}; and
(II) when T represents a single bond and Y represents —C(O)OR^{9b}, then D represents a single bond,
or a pharmaceutically-acceptable salt thereof,
provided that, when X¹ represents -Q-X², R², R⁴ and R⁵ all represent H, R³ represents -D-E, E represents unsubstituted phenyl, T represents a single bond, Y represents —C(O)OR^{9b}, R^{9b} represents ethyl, and R¹ represents 2,4-dinitrophenyl, then:
(a) when Q represents a single bond, X² does not represent methyl; and
(b) when Q represents —O—, X² does not represent methyl or ethyl,
   which compounds and salts are referred to hereinafter as “the compounds of the invention”.

According to a second aspect of the invention, there is a provided a compound of formula I as hereinbefore defined, or a pharmaceutically-acceptable salt thereof, provided that T does not represent a single bond when Y represents —C(O)OR^{9b}.

According to a third aspect of the invention, there is provided a compound of formula I as hereinbefore defined, or a pharmaceutically-acceptable salt thereof, in which T represents a single bond, Y represents —C(O)OR^{9b} and X¹ represents -Q-X² in which X² represents:
(a) C₁₋₈ alkyl or a heterocycloalkyl group, both of which are optionally substituted by one or more substituents selected from G¹ and/or Z¹;
(b) provided that Q does not represent a single bond, an aryl group or a heteroaryl group, both of which are optionally substituted by one or more substituents selected from A; or, when Q is a single bond;
(c) cyano.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl, and C_{1-q} alkylene, groups (where q is the upper limit of the range) defined herein may be straight-chain or, when there is a sufficient number (i.e. a minimum of two or three, as appropriate) of carbon atoms, be branched-chain, and/or cyclic (so forming, in the case of alkyl, a C_{3-q}-cycloalkyl group or, in the case of alkylene, a C_{3-q} cycloalkylene group). Further, when there is a sufficient number (i.e. a minimum of four) of carbon atoms, such groups may also be part cyclic. When one of the groups R² to R⁵ represents -D-E, and the other groups are C₁₋₈ alkyl, then it is preferred that such an alkyl group is not cyclic. Such alkyl and alkylene groups may also be saturated or, when there is a sufficient number (i.e. a minimum of two) of carbon atoms, be unsaturated (forming, for example, in the case of alkyl, a C_{2-q} alkenyl or a C_{2-q} alkynyl group or, in the case of alkylene, a C_{2-q} alkenylene or a C_{2-q} alkynylene group).

C_{3-q} cycloalkyl groups (where q is the upper limit of the range) that may be mentioned may be monocyclic or bicyclic alkyl groups, which cycloalkyl groups may further be bridged (so forming, for example, fused ring systems such as three fused cycloalkyl groups). Such cycloalkyl groups may be saturated or unsaturated containing one or more double or triple bonds (forming for example a C_{3-q} cycloalkenyl or a C_{8-q} cycloalkynyl group). Substituents may be attached at any point on the cycloalkyl group. Further in the case where the substituent is another cyclic compound, then the cyclic substituent may be attached through a single atom on the cycloalkyl group, forming a so-called “spiro”-compound.

C₂₋₈ heteroalkylene chains include C₂₋₈ alkylene chains that are interrupted by one or more heteroatom groups selected from —O—, —S— or —N(R²⁵)—, in which R²⁵ represents C₁₋₄ alkyl, optionally substituted by one or more halo (e.g. fluoro) groups.

The term “halo”, when used herein; includes fluoro, chloro, bromo and iodo.

Heterocycloalkyl groups that may be mentioned include non-aromatic monocyclic and bicyclic heterocycloalkyl groups (which groups may further be bridged) in which at least one (e.g. one to four) of the atoms in the ring system is other than carbon (i.e. a heteroatom), and in which the total number of atoms in the ring system is between three and twelve (e.g. between five and ten). Further, such heterocycloalkyl groups may be saturated or unsaturated containing one or more double and/or triple bonds, forming for example a C_{2-q} heterocycloalkenyl (where q is the upper limit of the range) or a C_{3-q} heterocycloalkynyl group. C_{2-q} heterocycloalkyl groups that may be mentioned include 7-azabicyclo-[2.2.1]heptanyl, 6-azabicyclo[3.1.1]hept-anyl, 6-azabicyclo[3.2.1]-octanyl, 8-azabicyclo[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo[3.2.1]-octanyl, oxetanyl, oxiranyl, piperazinyl, piperidinyl, pyranyl; pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like. Substituents on heterocycloalkyl groups may, where appropriate, be located on any atom in the ring system including a heteroatom. Further, in the case where the substituent is another cyclic compound, then the cyclic compound may be attached through a single atom on the heterocycloalkyl group, forming a so-called “spiro”-compound. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N- or S-oxidised form.

For the avoidance of doubt, the term “bicyclic”, when employed in the context of cycloalkyl and heterocycloalkyl groups refers to such groups in which the second ring is formed between two adjacent atoms of the first ring. The term “bridged”, when employed in the context of cycloalkyl or heterocycloalkyl groups refers to monocyclic or bicyclic groups in which two non-adjacent atoms are linked by either an alkylene or heteroalkylene chain (as appropriate).

Aryl groups that may be mentioned include C₆₋₁₄ (such as C₆₋₁₃ (e.g. C₆₋₁₀)) aryl groups. Such groups may be monocyclic or bicyclic and have between 6 and 14 ring carbon atoms, in which at least one ring is aromatic. C₆₋₁₄ aryl groups include phenyl, naphthyl and the like, such as 1,2,3,4-tetrahydronaphthyl, indanyl, indenyl and fluorenyl. The point of attachment of aryl groups may be via any atom of the ring system. However, when aryl groups are bicyclic or tricyclic, they are linked to the rest of the molecule via an aromatic ring.

Heteroaryl groups that may be mentioned include those which have between 5 and 14 (e.g. 10) members. Such groups may be monocyclic, bicyclic or tricyclic, provided that at least one of the rings is aromatic and wherein at least one (e.g. one to four) of the atoms in the ring system is other than carbon (i.e. a heteroatom). Heterocyclic groups that may be mentioned include benzothiadiazolyl (including 2,1,3-benzothiadiazolyl), isothiochromanyl and, more preferably, acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form.

Heteroatoms that may be mentioned include phosphorus, silicon, boron, tellurium, selenium and, preferably, oxygen, nitrogen and sulphur.

For the avoidance of doubt, “heterocycloalkylene”, “arylene”, “heteroarylene” and “cycloalkylene” groups as defined herein comprise “linking” groups in which a heterocycloalkyl, an aryl, a heteroaryl, or a cycloalkyl, group (each of which are as defined hereinbefore), serves the purpose of linking two different parts of a compound of the invention together, in exactly the same way as an alkylene group can be said to constitute a “linking” (i.e. a divalent) alkyl group. Thus, for example, a phenyl group that serves the purpose of linking two substituents within, or parts of, a compound of the invention together would be classified in the context of the present invention as a “phenylene” group.

For the avoidance of doubt, in cases in which the identity of two or more substituents in a compound of the invention may be the same, the actual identities of the respective substituents are not in any way interdependent. For example, in the situation in which R¹ and X² are both aryl groups substituted by one or more C₁₋₈ alkyl groups, the alkyl groups in question may be the same or different. Similarly, when groups are substituted by more than one substituent as defined herein, the identities of those individual substituents are not to be regarded as being interdependent. For example, when X² and/or R¹ represents e.g. an aryl group substituted by G¹ in addition to, for example, C₁₋₈ alkyl, which latter group is substituted by G¹, the identities of the two G¹ groups are not to be regarded as being interdependent.

For the avoidance of doubt, when a term such as “R^{9a} to R^{9x}” is employed herein, this will be understood by the skilled person to mean R^{9a}, R^{9b}, R^{9c}, R^{9d}, R^{9e}, R^{9f}, R^{9g}, R^{9h}, R⁹ⁱ, R^{9j}, R^{9k}, R^{9m}, R⁹ⁿ, R^{9p}, R^{9q}, R^{9r}, R^{9s}, R^{9t}, R^{9u}, R^{9v}, R^{9w} and R^{9x} inclusively.

Any pair of R^{9a} to R^{9x} and lea, R^{10a}, R^{10f}, R^{10g}, R¹⁰ⁱ or R^{10j}, may be linked together to form a ring as hereinbefore defined. Thus R^{9a} to R^{9x}, R^{10a}, R^{10f}, R^{10g}, R¹⁰ⁱ and R^{10j} groups may be attached to (a) a single nitrogen atom (e.g. R^{9f} and R^{10f}), or (b) a nitrogen atom and a J group (i.e. R^{9a} and R^{10a}), which also form part of the ring, or two R^{9a} to R^{9x} (e.g. two R^{9d}) groups may be attached to different oxygen atoms (for example in a 1,3-relationship) all of which may form part of the ring.

Compounds of the invention that may be mentioned include those in which Y represents —C(O)OR^{9b}, —S(O)₃R^{9c}, —P(O)(OR^{9d})₂, —P(O)(OR^{9e})N(R^{10f})R^{9f}, —P(O)(N(R^{10g})R^{9g})₂, —B(OR^{9h})₂, —C(CF₃)₂OH, —S(O)₂N(R¹⁰ⁱ)R⁹ⁱ or any one of the following groups:

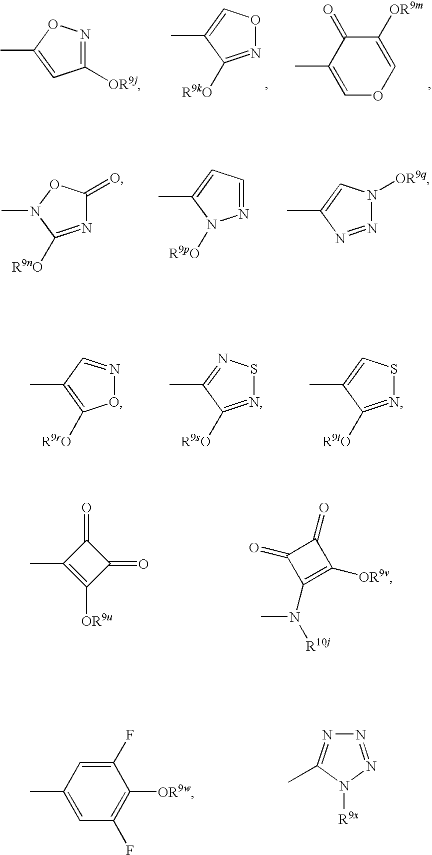

Further compounds of the invention that may be mentioned include those in which:

X² represents:
(a) C₁₋₅ alkyl or a heterocycloalkyl group, both of which are optionally substituted by one or more substituents selected from G¹ and/or Z¹; or
(b) an aryl group or a heteroaryl group, both of which are optionally substituted by one or more substituents selected from A.

Compounds of the invention that may be mentioned also include those in which, when X¹ is -Q-X² and Q is a single bond and X² is either:
(a) an aryl group or a heteroaryl group, which groups are substituted by A in which A is G¹; or
(b) C₁₋₈ alkyl or a heterocycloalkyl group, which groups are substituted by G¹, and G¹ is -A¹-R^{11a}, then A¹ represents a single bond or a spacer group selected from —C(O)—, —S(O)₂—, —S(O)₂N(R^{12c})—, —N(R^{12a})A⁴- or —OA⁵-.

Further compounds of the invention that may be mentioned include those in which, when X¹ is -Q-X² and Q is a single bond, X² is C₁₋₈ alkyl substituted by G¹, G¹ is -A¹-R^{11a}, A¹ is a single bond, R^{11a} represents an aryl group, a heteroaryl group or a heterocycloalkyl group, all of which groups are substituted by G³, and G³ is -A¹¹-R^{15a}, then A¹¹ represents a single bond or a spacer group selected from —C(O)—, —S(O)₂—, —S(O)₂NR^{16c})—, —N(R^{16a})A¹⁴- or —OA¹⁵.

Further compounds of the invention that may be mentioned include those in which when X¹ is -Q-X², Q is a single bond, and X² represents C₁₋₈ alkyl terminally substituted by both Z¹ and G¹, in which Z¹ represents ═O and G¹ represents -A¹-R^{11a}, then when A¹ represents —N(R^{12a})A⁴-, A⁴ represents —C(O)—, —C(O)N(R^{12d})—, —C(O)O—, or —S(O)₂N(R^{12e}), and when A¹ represents —OA⁵-, A⁵ represents —C(O)—, —C(O)N(R^{12d})—, —C(O)O—, —S(O)₂— or —S(O)₂N(R^{12e}).

Still further compounds the invention that may be mentioned include those in which when Y represents either:

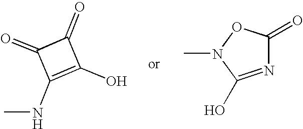

and T represents C₁₋₈ alkylene or C₂₋₈ heteroalkylene, both of which are substituted at the carbon atom that is adjacent to Y by Z¹, then Z¹ represents ═S, ═NOR^{11b}, ═NS(O)₂N(R^{12f})R^{11c}, ═NCN or ═C(H)NO₂.

Preferred compounds of the first and second aspects of the invention include those in which:

X² represents C₁₋₆ (e.g. C₁₋₄) alkyl or heterocycloalkyl, both of which groups are optionally substituted by one or more (e.g. one) groups selected from G¹ and/or Z¹;
R^{9a} to R^{9x} independently represent H or C₁₋₆ alkyl;
R^{10a}, R^{10f}, R^{10g}, R¹⁰ⁱ and R^{10j} independently represent H or C₁₋₆ (e.g. C₁₋₃) alkyl, which latter group is optionally substituted by one or more (e.g. one) groups selected from G¹;
or any pair of R^{9a} to R^{9x} and R^{10a}, R^{10f}, R^{10g}, R¹⁰ⁱ or R^{10j} are linked to form a 4- to 7-membered (e.g. 5- or 6-membered) ring, which ring may, for example preferably, contain (in addition to the nitrogen atom to which R^{9a} to R^{9x} is attached) a further heteroatom (e.g. nitrogen or oxygen) and which ring is optionally substituted by one or more Z¹ groups;
J represents a single bond, —C(O)— or —S(O)₂—.

Preferred compounds of the first and third aspects of the invention include those in which:

X² represents a heterocycloalkyl group, or a C₁₋₇ alkyl group, both of which are optionally substituted with one or more G¹ and/or Z¹ substituents.

Preferred compounds of the invention include those in which:

A represents G¹ or C₁₋₇ alkyl, more preferably, (particularly in the case of compounds of the third aspect of the invention) C₁₋₆ alkyl, which alkyl group is optionally substituted by one or more G¹ groups;
G¹ represents cyano, —NO₂ or (more preferably in the case of compounds of the second aspect of the invention) halo or -A¹-R^{11a};
A¹ represents a single bond, —C(O)A²-, —N(R^{12a})A⁴- or —OA⁵- and, more preferably, (in the case of compounds of the third aspect of the invention) a single bond, —N(R^{12a})A⁴- or —OA⁵- and (in the case of compounds of the second aspect of the invention) —OA⁵-;
A² represents —O—;
A⁴ and A⁵ independently represent —C(O)—, —C(O)N(R^{12d})—, —C(O)O— or (more preferably in the case of compounds of the second aspect of the invention) a single bond;
R^{11a}, R^{11b} and R^{11c} independently represent H, a heterocycloalkyl group (such as C₄₋₈ heterocycloalkyl, which group contains one oxygen or, preferably, nitrogen atom and, optionally, a further nitrogen or oxygen atom, and which heterocycloalkyl group is optionally substituted by one or more G³ and/or Z³ groups) or a heteroaryl group (which heteroaryl group is optionally substituted by one or more G³ groups) or, in the case of compounds of the second aspect of the invention, C₁₋₆ alkyl, which alkyl group is optionally substituted .by one or more G³ and/or Z³ groups;
R^{12a}, R^{12b}, R^{12c}, R^{12d}, R^{12e} and R^{12f} independently represent H or (preferably in the case of compounds of the second aspect of the invention) C₁₋₃ (e.g. C₁₋₂) alkyl;
or, for example, in the case of compounds of the third aspect of the invention, any pair of R^{11a} to R^{11c} and R^{12a} to R^{12f}, together with the atom(s) to which they are attached, represent a nitrogen-containing heterocycloalkyl group optionally substituted by one or more G³ and/or Z³ groups;
Z¹ represents ═NOR^{11b}, ═NCN or, preferably, ═O;
G² represents cyano, —N₃ or, more preferably, halo, —NO₂ or -A⁶-R^{13a};
A⁶ represents —N(R^{14a})A⁹- or —OA¹⁰-;
A⁹ represents —C(O)N(R^{14d})—, —C(O)O— or, more preferably, a single bond or —C(O)—;
A¹⁰ represents a single bond;
Z² represents ═NOR^{13b}, ═NCN or, more preferably, ═O;
R^{13a}, R^{13b}, R^{13c}, R^{14a}, R^{14b}, R^{14c}, R^{14d}, R^{14e} and R^{14f} independently represent H or C₁₋₃ alkyl;
G³ represents halo, —NO₂ or -A¹¹-R^{15a};
A¹¹ represents —N(R^{16a})A¹⁴- or —OA¹⁵- or, particularly so in the case of compounds of the third aspect of the invention, a single bond or —C(O)A^{l2}-,
A¹² represents —O—;
A¹⁴ and A¹⁵ independently represent a single bond;
R^{15a}, R^{15b} and R^{15c} independently represent H, C₁₋₃ alkyl or heteroaryl;
R^{16a}, R^{16b}, R^{16c}, R^{16d}, R^{16e} and R^{16f} independently represent H or C₁₋₃ alkyl;
Z³ represents ═O;
when any one of R^{15a}, R^{15b}, R^{15c}, R^{16a}, R^{16b}, R^{16c}, R^{16d}, R^{16e} and R^{16f} represents optionally substituted C₁₋₆ alkyl, the optional substituent is one or more halo groups;
when any one of R^{17a}, R^{17b}, R^{17c}, R^{17d}, R^{17e}, R^{17f}, R^{18a}, R^{18b} and R^{18c} represents optionally substituted C₁₋₄ alkyl, the optional substituent is one or more fluoro groups.

Preferred aryl and heteroaryl groups that R¹, E, and X² (when X² represents an aryl or heteroaryl group) may represent include optionally substituted phenyl, naphthyl, pyrrolyl, furanyl, thienyl, pyrazolyl, imidazolyl (e.g 1-imidazolyl, 2-imidazolyl or 4-imidazolyl), oxazolyl, isoxazolyl, thiazolyl, pyridyl (e.g. 2-pyridyl, 3-pyridyl or 4-pyridyl), indazolyl, indolyl, indolinyl, isoindolinyl, quinolinyl, 1,2,3,4-tetrahydroquinolinyl, 5,6,7,8-tetrahydroquinolinyl, isoquinolinyl, 1,2,3,4-tetrahydroisoquinolinyl, 5,6,7,8-tetrahydroiso-quinolinyl, quinolizinyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, and/or benzodioxanyl, groups.

Preferred values of R¹ include optionally substituted phenyl, pyridyl and imidazolyl.

Preferred values of E (for example, in compounds of the second aspect of the invention) include optionally substituted phenyl, pyridyl and imidazolyl.

Preferred values of R², R⁴, R⁵ and, particularly, R³ (for example in compounds of the third aspect of the invention) include optionally substituted phenyl, pyridyl (e.g. 2-pyridyl), tetrahydroquinolinyl (e.g. 5,6,7,8-tetrahydroquinolin-2-yl) or imidazolyl (e.g. 4-imidazolyl).

Optional substituents on R¹, X² (particularly so in the case of compounds of the third aspect of the invention, when X² represents an aryl or heteroaryl group) and E groups are preferably selected from:

halo (e.g. fluoro, chloro or bromo);
cyano;

### —NO₂;

C₁₋₆ alkyl, which alkyl group may be linear or branched (e.g. C₁₋₄ alkyl (including ethyl, n-propyl, isopropyl, n-butyl or, preferably, methyl or t-butyl), n-pentyl, isopentyl, n-hexyl or isohexyl), cyclic (e.g. cyclopropyl, cyclobutyl, cyclopentyl or cyclohexyl), part-cyclic (e.g. cyclopropylmethyl), unsaturated (e.g. 1-propenyl, 2-propenyl, 1-butenyl, 2-butenyl, 3-butenyl, 1-pentenyl, 2-pentenyl, 4-pentenyl or 5-hexenyl) and/or optionally substituted with one or more halo (e.g. fluoro) group (so forming, for example, fluoromethyl, difluoromethyl or, preferably, trifluoromethyl);
heterocycloalkyl, such as a C₄₋₅ heterocycloalkyl group, preferably containing a nitrogen atom and, optionally, a further nitrogen or oxygen atom, so forming for example morpholinyl (e.g. 4-morpholinyl), piperazinyl (e.g. 4-piperazinyl) or piperidinyl (e.g. 1-piperidinyl and 4-piperidinyl) or pyrrolidinyl (e.g. 1-pyrrolidinyl), which heterocycloalkyl group is optionally substituted by one or more (e.g. one or two) substituents selected from C₁₋₃ alkyl (e.g. methyl) and ═O; —OR¹⁹; and

### —N(R¹⁹)R²⁰;

wherein R¹⁹ and R²⁰ independently represent, on each occasion when mentioned above, H or C₁₋₆ alkyl, such as, in the case of compounds of the third aspect of the invention, ethyl, n-propyl, n-butyl, 1-butyl or, preferably, methyl or isopropyl (which alkyl groups are optionally cyclic (e.g. cyclopentyl or cyclohexyl) and/or are optionally substituted by one or more halo (e.g. fluoro) groups (to form e.g. a trifluoromethyl group)), or, in the case of compounds of the second aspect of the invention, methyl, ethyl, n-propyl, n-butyl, t-butyl, cyclopropyl, cyclobutyl, cyclohexyl or, preferably, isopropyl or cyclopentyl (which alkyl groups are optionally substituted by one or more halo (e.g. fluoro) groups (to form e.g. a trifluoromethyl group)).

When X² represents C₁₋₇ alkyl or a heterocycloalkyl group, optional substituents on such groups are preferably selected from:

halo (e.g. fluoro or chloro);
cyano;

### ═O;

a heterocycloalkyl group, such as a 4- to 8-membered heterocycloalkyl group containing one nitrogen atom and, optionally, a further nitrogen and or oxygen atom (which heterocycloalkyl group may be optionally further substituted by one or more substituents selected from ═O and C₁₋₃ alkyl, which alkyl group is itself optionally substituted by one or more fluoro groups);
a heteroaryl group, such as a 5- or 6-membered heteroaryl group;

### —OR²¹; and

### —N(R²¹)R²²;

wherein R²¹ represents H or C₁₋₆ (e.g. C₁₋₃) alkyl, such as ethyl or, preferably, methyl; and
R²² represents H or, preferably, C₁₋₆ (e.g. C₁₋₃) alkyl (e.g. methyl, ethyl or isopropyl), which latter group is optionally substituted by one or two substituents selected from —OR²³ and —N(R²³)R²⁴, in which R²³ and R²⁴ independently represents H or C₁₋₃ alkyl (e.g. methyl).

Such compounds are particularly preferred in the case of compounds of the third aspect of the invention.

Preferred values of R^{9a} to R^{9x} include C₁₋₄ alkyl (e.g. particularly so for compounds of the second aspect of the invention, ethyl) and, particularly, H. Preferred values (e.g. particularly so for compounds of the second aspect of the invention) of lea, R^{10a}, R^{10f}, R^{10g}, R¹⁰ⁱ and R^{10j} include C₁₋₃ alkyl and H.

More preferred compounds include those in which:

one of R⁴ and, more preferably, R³ represent an optionally substituted aryl or heteroaryl group and the other (more preferably) represents H;
R² and/or R⁵ represent H;
X² represents cyano, or more preferably, a 5- or 6-membered nitrogen-containing heterocycloalkyl group (e.g. piperidinyl, such as piperidin-3-yl), or optionally unsaturated linear, branched or cyclic C₁₋₆ alkyl (e.g. n-propyl, t-butyl or, preferably, methyl, ethyl, ethenyl, isopropyl, cyclopentyl or cyclohexyl), which latter two groups are optionally substituted with one or more G¹ and/or Z¹ substituents;
Q represents —C(O)—, —S(O)— or —S(O)₂— or, preferably, —O—, —S— or, more preferably, a single bond;
A represents G¹ or optionally branched C₁₋₄ alkyl (e.g. methyl or t-butyl) optionally substituted by one or more G¹ groups;
G¹ represents halo (e.g. fluoro or chloro), cyano or -A¹-R^{11a};
A¹ represents a single bond, —N(R^{12a})A⁴- or —OA⁵-;
A⁴ and A⁵ independently represent a single bond;
Z¹ represents ═O;
R^{11a}, R^{11b} and R^{11c} independently represent H or, preferably, a heteroaryl group (such as tetrazolyl (e.g. 5-tetrazolyl), imidazolyl (e.g. 4-imidazolyl and/or 2-imidazolyl) or, more preferably, pyridyl (e.g. 2-pyridyl, 3-pyridyl and, especially, 4-pyridyl) or thiazolyl (e.g. 5-thiazolyl)), an optionally branched, optionally unsaturated and/or optionally cyclic C₁₋₆ alkyl group (e.g. n-propyl, n-butyl, t-butyl, n-pentyl or, preferably, methyl, ethyl, isopropyl or cyclopentyl), both of which groups are optionally substituted by one or more G³ groups;
R^{12a}, R^{12b}, R^{12c}, R^{12d}, R^{12e} and R^{12f} independently represent H or C₁₋₂ alkyl (e.g. methyl);
when A¹ represents —N(R^{12a})A⁴- and A⁴ represents a single bond, R^{11a} and R^{12a}, together with the nitrogen to which they are both attached, represent a 5- to 7-membered nitrogen-containing heterocycloalkyl group (which heterocycloalkyl group optionally contains a further nitrogen or oxygen atom so forming, for example, a morpholinyl (e.g. 1-morpholinyl) or a piperazinyl (e.g. 1-piperazinyl) group), optionally substituted by one or more G³ and/or ═O groups;
G³ represents -A¹¹-R^{15a};
A¹¹ represents a single bond, —N(R^{16a})— or —O—;
R^{15a}, R^{15b} and R^{15c} independently represent H, C₁₋₂ alkyl (e.g. methyl) or a nitrogen-containing heteroaryl group (e.g. pyridyl, such as 2-pyridyl);
R^{16a}, R^{16b}, R^{16c}, R^{16d}, R^{16e} and R^{16f} independently represent C₁₋₂ alkyl (e.g. methyl).

Such compounds are particularly preferred in the case of compounds of the third aspect of the invention.

More preferred compounds also include those in which:

T represents C₂₋₄ heteroalkylene (e.g. C₂ heteroalkylene interrupted by —N(R²⁵)— in which R²⁵ represents C₁₋₂ alkyl (e.g. methyl)) or, preferably, a single bond or linear or branched C₁₋₅ (e.g. C₁₋₄) alkylene (such as ethylene (e.g. ethenylene)), which latter group is optionally substituted by one or more (e.g. one) Z¹ substituent;
Y represents —C(O)OR^{9b}, —B(OR^{9b})₂, —S(O)₃R^{9c}, —P(O)(OR^{9d})₂, —S(O)₂N(R¹⁰ⁱ)R⁹ⁱ or a tetrazolyl group (e.g. a 1H-tetrazol-5-yl group);
one of R⁴ and, more preferably, R³ represents -D-E and the other (more preferably) represents H;
D represents a single bond or —O—;
R² and/or R⁵ represent H;
X¹ represents halo (e.g. chloro or fluoro), -Q-X² or H;
Q represents —O—, —S—, and, in particular, a single bond;
X² represents C₁₋₃ alkyl (e.g. methyl) or heterocycloalkyl, both of which are optionally substituted by one or more G¹ groups;
A represents G¹ or C₁₋₆ alkyl (e.g. methyl, t-butyl or cyclohexyl) optionally substituted by one or more G¹ groups;
G¹ represents fluoro, chloro or -A¹-R^{11a};
A⁴ and A⁵ independently represent a single bond;
R^{11a}, R^{11b} and R^{11c} independently represent a heteroaryl group (such as tetrazolyl (e.g. 5-tetrazolyl), imidazolyl (e.g. 4- or 2-imidazolyl) or pyridyl (e.g. 2-pyridyl, 3-pyridyl or 4-pyridyl) or a C₄₋₅ heterocycloalkyl group (e.g. pyrrolidinyl, piperidinyl, piperazinyl and morpholinyl) or, more preferably, C₁₋₅ alkyl (e.g. methyl, isopropyl or cyclopentyl), all of which are optionally substituted by one or more G³ groups;
R^{12a}, R^{12b}, R^{12c}, R^{12d}, R^{12e} and R^{12f} independently represent H or methyl;
G³ represents halo (e.g. fluoro).

Such compounds are particularly preferred in the case of compounds of the second aspect of the invention.

Preferred values of X² include cyano or, preferably, C₁₋₄ (e.g. C₁₋₃) alkyl (e.g. t-butyl or, preferably, n-propyl, isopropyl, ethyl, ethenyl, or, more preferably, methyl), which group is unsubstituted or, preferably, substituted by one or more cyano, ═O, morpholinyl, piperazinyl, (e.g. 4-methylpiperazinyl), —NH₂, —N(CH₃)₂, —N(H)C₂H₄OH, —N(H)CH(CH₂OH)₂, —N(H)CH₂-pyrid-2-yl, —N(H)C₂H₄N(CH₃)₂, thiazolyl (e.g. 4-methylthiazol-5-yl), 2-pyridyl, 4-pyridyl or, more preferably, halo (e.g. fluoro or chloro) groups so forming, for example, a trifluoromethyl group. Such compounds are particularly preferred in the case of compounds of the third aspect of the invention.

Particularly preferred values of R¹ in the compounds of the invention include 4-isopropoxyphenyl, 4-cyclopentoxyphenyl and 4-cyclopropoxyphenyl.

Particularly preferred values of E (e.g. R³, when R³ represents -D-E and D represents a single bond) include 4-tert-butylphenyl, 4-trifluoromethylphenyl, 5-trifluoromethylpyrid-2-yl, 4-trifluormethoxyphenyl, 3-trifluoromethoxy-4-chlorophenyl and 3-trifluoromethoxy-4-isopropoxyphenyl.

Particularly preferred compounds of the invention include those of the examples described hereinafter.

Compounds of the invention may be made in accordance with techniques that are well known to those skilled in the art, for example as described hereinafter.

According to a further aspect of the invention there is provided a process for the preparation of a compound of formula I which process comprises:

(i) reaction of a compound of formula II,

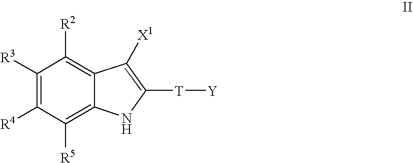

wherein X¹, R², R³, R⁴, R⁵, T and Y are as hereinbefore defined, with a compound of formula III,

R¹L¹ III

wherein L¹ represents a suitable leaving group such as chloro, bromo, iodo, a sulfonate group (e.g. —OS(O)₂CF₃, —OS(O)₂CH₃, —OS(O)₂PhMe or a nonaflate) or —B(OH)₂ and R¹ is as hereinbefore defined, for example optionally in the presence of an appropriate metal catalyst (or a salt or complex thereof) such as Cu, Cu(OAc)₂, CuI (or CuI/diamine complex), Pd(OAc)₂, Pd₂(dba)₃ or NiCl₂ and an optional additive such as Ph₃P, 2,2′-bis(diphenylphosphino)-1,1′-binaphthyl, xantphos, NaI or an appropriate crown ether such as 18-crown-6-benzene, in the presence of an appropriate base such as NaH, Et₃N, pyridine, N,N′-dimethylethylenediamine, Na₂CO₃, K₂CO₃, K₃PO₄, Cs₂CO₃, t-BuONa or t-BuOK (or a mixture thereof), in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof) or in the absence of an additional solvent when the reagent may itself act as a solvent (e.g. when R¹ represents phenyl and L¹ represents bromo, i.e. bromobenzene). This reaction may be carried out at room temperature or above (e.g. at a high temperature, such as the reflux temperature of the solvent system that is employed) or using microwave irradiation;
(ii) for compounds of formula I in which X¹ represents -Q-X², in which Q is a single bond or —C(O)—, reaction of a compound of formula IV,

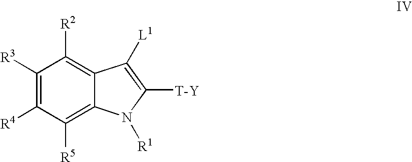

wherein L¹, R¹, R², R³, R⁴, R⁵, T and Y are as hereinbefore defined, with a compound of formula V,

X²-Q^{a}-L² V

wherein Q^{a} represents a single bond or —C(O)—, L² represents a suitable leaving group such as chloro, bromo, iodo, —B(OH)₂ or a protected derivative thereof, for example a 4,4,5,5-tetramethyl-1,3,2-dioxaborolan-2-yl group, 9-borabicyclo-[3.3.1]nonane (9-BBN), —Sn(alkyl)₃ (e.g. —SnMe₃ or —SnBu₃), or a similar group known to the skilled person, and X² is as hereinbefore defined. The skilled person will appreciate that L¹ and L² will be mutually compatible. In this respect, preferred leaving groups for compounds of formula V in which Q^{a} is —C(O)— include chloro or bromo groups, and preferred leaving groups for compounds of formula V in which Q^{a} is a single bond include —B(OH)₂, 4,4,5,5-tetramethyl-1,3,2-dioxaborolan-2-yl, 9-borabicyclo[3.3.1]nonane or —Sn(alkyl)₃. This reaction may be performed, for example in the presence of a suitable catalyst system, e.g. a metal (or a salt or complex thereof) such as CuI, Pd/C, PdCl₂, Pd(OAc)₂, Pd(Ph₃P)₂Cl₂, Pd(Ph₃P)₄, Pd₂(dba)₃ or NiCl₂ and a ligand such as t-Bu₃P, (C₆H₁₁)₃P, Ph₃P, AsPh₃, P(o-Tol)₃, 1,2-bis(diphenylphosphino)ethane, 2,2′-bis(di-tert-butylphosphino)-1,1′-bi-phenyl, 2,2′-bis(diphenylphosphino)-1,1′-bi-naphthyl, 1,1′-bis(diphenylphosphinoferrocene), 1,3-bis(diphenylphosphino)-propane, xantphos, or a mixture thereof, together with a suitable base such as, Na₂CO₃, K₃PO₄, Cs₂CO₃, NaOH, KOH, K₂CO₃, CsF, Et₃N, (i-Pr)₂NEt, t-BuONa or t-BuOK (or mixtures thereof) in a suitable solvent such as dioxane, toluene, ethanol, dimethylformamide, ethylene glycol dimethyl ether, water, dimethylsulfoxide, acetonitrile, dimethylacetamide, N-methylpyrrolidinone, tetrahydrofuran or mixtures thereof. The reaction may also be carried out for example at room temperature or above (e.g. at a high temperature such as the reflux temperature of the solvent system) or using microwave irradiation. In the case where Q^{a} represents a single bond and X² represents either C₂₋₈ alkenyl, cycloalkenyl or heterocycloalkenyl in which the double bond is between the carbon atoms that are α and β to L², the skilled person will appreciate that the double bond may migrate on formation of the compound of formula I to form a double bond that is between the carbon atoms that are β and γ to the indole ring;
(iii) for compounds of formula I in which X¹ represents -Q-X² and Q represents —C(O)—, reaction of a compound of formula I in which X¹ represents H, with a compound of formula V in which Q^{a} represents —C(O)— and L² represents a suitable leaving group such as chloro or bromo, —N(C₁₋₆ alkyl)₂ (e.g. —N(CH₃)₂) or a carboxylate group such as —O—C(O)—X^{2y} in which X^{2y} represents X² or H. In the latter case, X^{2y} and X² are preferably the same, or X^{2y} represents e.g. H, CH₃ or CF₃. This reaction may be performed under suitable conditions known to those skilled in the art, for example in the presence of a suitable Lewis acid (e.g. AlCl₃ or FeCl₃). Reaction of a compound of formula V in which L² represents —N(C₁₋₆ alkyl)₂ and X² represents optionally substituted aryl (e.g. phenyl) or heteroaryl, the reaction may be performed in the presence of a reagent such as POCl₃, for example under reaction conditions described in Bioorg. Med. Chem. Lett., 14, 4741-4745 (2004). The skilled person will appreciate that in the latter instance, POCl₃ may convert the compound of formula V into one in which L² represents chloro and/or Q^{a} represents a derivative of —C(O)— (e.g. an iminium derivative), which group may be transformed back to a —C(O)— group before or after reaction with the compound of formula I in which X¹ represents H;
(iv) for compounds of formula I in which X¹ represents —N(R^{9a})-J-R^{10a} or -Q-X² in which Q represents —O— or —S—, reaction of a compound of formula IV as hereinbefore defined with a compound of formula VI,

X^{1b}H VI

in which X^{1b} represents —N(Z^{9a})-J-R^{10a} or -Q-X² in which Q represents —O— or —S— and R^{9a}, J, R^{10a} and X² are as hereinbefore defined, for example under reaction conditions as hereinbefore described in respect of either process (i) or (ii) above;
(v) for compounds of formula I in which X¹ represents -Q-X² and Q represents —S—, reaction of a compound of formula I in which X¹ represents H, with a compound of formula VI in which X^{1b} represents -Q-X², Q represents —S— and X² is as hereinbefore defined, for example in the presence of N-chlorosuccinimide and a suitable solvent (e.g. dichloromethane), e.g. as described in inter alia Org. Lett., 819-821 (2004). Alternatively, reaction with a compound of formula VI in which X^{1b} represents -Q-X², Q represents —S— and X² represents an optionally substituted aryl (phenyl) or heteroaryl (e.g. 2-pyridyl) group, may be performed in the presence of PIFA (PhI(OC(O)CF₃)₂) in a suitable solvent such as (CF₃)₂CHOH. Introduction of such an —S—X² group is described in inter alia Bioorg. Med. Chem. Lett., 14, 4741-4745 (2004);
(vi) for compounds of formula I in which X¹ represents -Q-X² and Q represents —S(O)— or —S(O)₂—, oxidation of a corresponding compound of formula I in which Q represents —S— under appropriate oxidation conditions, which will be known to those skilled in the art;
(vii) for compounds of formula I in which X¹ represents -Q-X², X² represents C₁₋₈ alkyl substituted by G¹, G¹ represents -A¹-R^{11a}, A¹ represents —N(R^{12a})A⁴- and A⁴ is a single bond (provided that Q represents a single bond when X² represents substituted C₁ alkyl), reaction of a compound of formula VII,

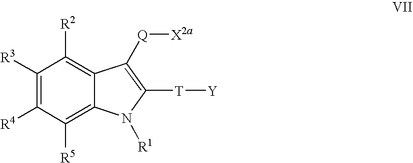

wherein X^{2a} represents a C₁₋₈ alkyl group substituted by a —Z¹ group in which Z¹ represents ═O, Q is as hereinbefore defined, provided that it represents a single bond when X^{2a} represents C₁ alkyl substituted by ═O (i.e. —CHO), and R¹, R², R³, R⁴, R⁵, T and Y are as hereinbefore defined, under reductive amination conditions in the presence of a compound of formula VIII,

R^{11a}(R^{12a})NH VIII

wherein R^{11a} and R^{12a} are as hereinbefore defined, under conditions well known to those skilled in the art;
(viia) for compounds of formula I in which X¹ represents -Q-X², Q represents a single bond, X² represents methyl substituted by G¹, G¹ represents -A¹-R^{11a}, A¹ represents —N(R^{12a})A⁴-, A⁴ is a single bond and R^{11a} and R^{12a} are preferably methyl, reaction of a corresponding compound of formula I in which X¹ represents H, with a mixture of formaldehyde (or equivalent reagent) and a compound of formula VIII as hereinbefore defined (e.g. in which R^{11a} and R^{12a} represent methyl), for example in the presence of solvent such as a mixture of acetic acid and water, under e.g. standard Mannich reaction conditions known to those skilled in the art;
(viii) for compounds of formula I in which X¹ represents -Q-X², Q represents a single bond and X² represents optionally substituted C₂₋₈ alkenyl (in which a point of unsaturation is between the carbon atoms that are É and é to the indole ring), reaction of a corresponding compound of formula IV in which L¹ represents halo (e.g. iodo) with a compound of formula IXA,

H₂C═C(H)X^{2b} IXA

or, depending upon the geometry of the double bond, reaction of a compound of formula VII in which Q represents a single bond and X^{2a} represents —CHO with either a compound of formula IXB,

(EtO)₂P(O)CH₂X^{2b} IXB

or the like, or a compound of formula IXC,

(Ph)₃P═CHX^{2b} IXC

or the like, wherein, in each case, X^{2b} represents H, G¹ or C₁₋₆ alkyl optionally substituted with one of more substituents selected from G¹ and/or Z¹ and G¹ and Z¹ are as hereinbefore defined, for example, in the case of a reaction of a compound of formula IV with compound of formula IXA, in the presence of an appropriate catalyst (such as PdCl₂(PPh₃)₂), a suitable base (e.g. NaOAc and/or triethylamine) and an organic solvent (e.g. DMF) and, in the case of reaction of a compound of formula VII with either a compound of formula IXB, or IXC, under standard Horner-Wadsworth-Emmons, or Wittig, reaction conditions, respectively;
(ix) for compounds of formula I in which X¹ represents -Q-X² and X² represents optionally substituted, saturated C₂₋₈ alkyl, saturated cycloalkyl, saturated heterocycloalkyl, C₂₋₈ alkenyl, cycloalkenyl or heterocycloalkenyl, reduction (e.g. hydrogenation) of a corresponding compound of formula I in which X² represents optionally substituted C₂₋₈ alkenyl, cycloalkenyl, heterocycloalkenyl, C₂₋₈ alkynyl, cycloalkynyl or heterocycloalkynyl (as appropriate) under conditions that are known to those skilled in the art. For example, in the case where an alkynyl group is converted to a alkenyl group, in the presence of an appropriate poisoned catalyst (e.g. Lindlar's catalyst);
(x) for compounds of formula I in which D represents a single bond, —C(O)—, —C(R⁷)(R⁸)—, C₂₋₄ alkylene or —S(O)₂—, reaction of a compound of formula X,

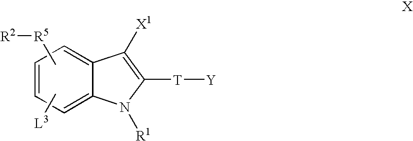

wherein L³ represents L¹ or L² as hereinbefore defined, which group is attached to one or more of the carbon atoms of the benzenoid ring of the indole, R²-R⁵ represents whichever of the three other substituents on the benzenoid ring, i.e. R², R³, R⁴ and R⁵, are already present in that ring, and X¹, R¹, R², R³, R⁴, R⁵, T and Y are as hereinbefore defined, with a compound of formula XI,

E-D^{a}-L⁴ XI

wherein D^{a} represents a single bond, —C(O)—, —C(R⁷)(R⁸)—, C₂₋₄ alkylene or —S(O)₂—, L⁴ represents L¹ (when L³ is L²) or L² (when L³ is L¹), and L¹, L², E, R⁷ and R⁸ are as hereinbefore defined. For example, when D^{a} represents a single bond, —C(O)— or C₂₋₄ alkylene, the reaction may be performed for example under similar conditions to those described hereinbefore in respect of process step (ii) above. Further, when D^{a} represents —C(O)—, —C(R⁷)(R⁸)—, C₂₋₄ alkylene or —S(O)₂—, the reaction may be performed by first activating the compound of formula X. The skilled person will appreciate that when L³ represents halo, compounds of formula X may first be activated by:
(I) forming the corresponding Grignard reagent under standard conditions known to those skilled in the art (e.g. employing magnesium or a suitable reagent such as a mixture of C₁₋₆ alkyl-Mg-halide and ZnCl₂ or LiCl), followed by reaction with a compound of formula XI, optionally in the presence of a catalyst (e.g. FeCl₃) under conditions known to those skilled in the art; or
(II) forming the corresponding lithiated compound under halogen-lithium exchange reaction conditions known to those skilled in the art (e.g. employing n-BuLi or t-BuLi in the presence of a suitable solvent (e.g. a polar aprotic solvent, such as THF)), followed by reaction with a compound of formula XI.

The skilled person will also appreciate that the magnesium of the Grignard reagent or the lithium of the lithiated species may be exchanged to a different metal (i.e. a transmetallation reaction may be performed), for example to zinc (e.g. using ZnCl₂) and the intermediate so formed may then be subjected to reaction with a compound of formula XI under conditions known to those skilled in the art, for example such as those described hereinbefore in respect of process (ii) above;

(xi) for compounds of formula I in which D represents —S—, —O— or C₂₋₄ alkynylene in which the triple bond is adjacent to E, reaction of a compound of formula X as hereinbefore defined in which L³ represents L² as hereinbefore defined (for example —B(OH)₂) with a compound of formula XII,

E-D^{b}-H XII

wherein D^{b} represents —S—, —O— or C₂₋₄ alkynylene in which the triple bond is adjacent to E and E is as hereinbefore defined. Such reactions may be performed under similar conditions to those described hereinbefore in respect of process step (ii) above, for example in the presence of a suitable catalyst system, such as Cu(OAc)₂, a suitable base, such as triethylamine or pyridine, and an appropriate organic solvent, such as DMF or dichloromethane;
(xii) for compounds of formula I in which D represents —S(O)— or —S(O)₂—, oxidation of a corresponding compound of formula I in which D represents —S— under appropriate oxidation conditions, which will be known to those skilled in the art;
(xiii) for compounds of formula I in which D represents —O— or —S—, reaction of a compound of formula XIII,

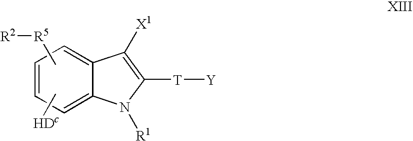

wherein the -D^{c}-H group is attached to one or more of the carbon atoms of the benzenoid ring of the indole, D^{c} represents —O— or —S—, and X¹, R¹, R²-R⁵, T and Y are as hereinbefore defined, with a compound of formula XIV,

E-L² XIV

wherein L² is as hereinbefore defined (for example —B(OH)₂, chloro, bromo or iodo) and E is as hereinbefore defined, for example under conditions such as those described hereinbefore in respect of process step (ii) above;
(xiv) for compounds of formula I in which X¹ represents —N(R^{9a})-J-R^{10a}, reaction of a compound of formula XV,

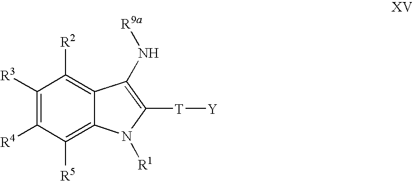

wherein R¹, R², R³, R⁴, R⁵, T, Y and R^{9a} are as hereinbefore defined, with a compound of formula XVI,

R^{10a}-J-L¹ XVI

wherein J, R^{10a} and L¹ are as hereinbefore defined, for example at around room temperature or above (e.g. up to 60-70° C.) in the presence of a suitable base (e.g. pyrrolidinopyridine, pyridine, triethylamine, tributylamine, trimethylamine, dimethylaminopyridine, diisopropylamine, 1,8-diazabicyclo[5.4.0]undec-7-ene, sodium hydroxide, or mixtures thereof) and an appropriate solvent (e.g. pyridine, dichloromethane, chloroform, tetrahydrofuran, dimethylformamide, dimethylsulfoxide, water, triethylamine or mixtures thereof) and, in the case of biphasic reaction conditions, optionally in the presence of a phase transfer catalyst;
(xv) for compounds of formula I in which X¹ represents —N(R^{9a})-J-R^{10a}, J represents a single bond and R^{10a} represents a C₁₋₈ alkyl group, reduction of a corresponding compound of formula I, in which J represents —C(O)— and R^{10a} represents H or a C₁₋₇ alkyl group, in the presence of a suitable reducing agent. A suitable reducing agent may be an appropriate reagent that reduces the amide group to the amine group in the presence of other functional groups (for example an ester or a carboxylic acid). Suitable reducing agents include borane and other reagents known to the skilled person;
(xvi) for compounds of formula I in which X¹ represents halo, reaction of a compound of formula I wherein X¹ represents H, with a reagent or mixture of reagents known to be a source of halide atoms. For example, for bromide atoms, N-bromosuccinimide, bromine or 1,2-dibromotetrachloroethane may be employed, for iodide atoms, iodine, diiodoethane, diiodotetrachloroethane or a mixture of NaI or KI and N-chlorosuccinimide may be employed, for chloride atoms, N-chlorosuccinimide may be employed and for fluoride atoms, 1-(chloromethyl)-4-fluoro-1,4-diazoniabicyclo[2.2.2]octane bis(tetrafluoroborate), 1-fluoropyridinium triflate, xenon difluoride, CF₃OF or perchloryl fluoride may be employed. This reaction may be carried out in a suitable solvent (e.g. acetone, benzene or dioxane) under conditions known to the skilled person;
(xvii) for compounds of formula I in which T and Y are as hereinbefore defined, provided that when Y represents —C(O)OR^{9b}, —S(O)₃R^{9c}, —P(O)(OR^{9d})₂, —P(O)(OR^{9e})N(R^{10f})R^{9f}, —P(O)(N(R^{10g})R^{9g})₂, —B(OR^{9h})₂ or —S(O)₂N(R¹⁰ⁱ)R⁹ⁱ, R^{9b} to R⁹ⁱ, R^{10f}, R^{10g} and R¹⁰ⁱ are other than H, reaction of a compound of formula XVII,

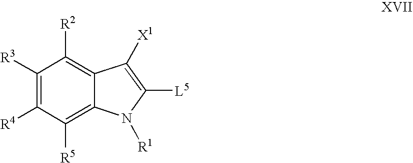

wherein L⁵ represents an appropriate alkali metal group (e.g. sodium, potassium or, especially, lithium), a —Mg-halide, a zinc-based group or a suitable leaving group such as halo or —B(OH)₂, or a protected derivative thereof (the skilled person will appreciate that the compound of formula XVII in which L⁵ represents an alkali metal (e.g. lithium), a Mg-halide or a zinc-based group may be prepared from a corresponding compound of formula XVII in which L⁵ represents halo, for example under conditions such as those hereinbefore described in respect of preparation of compounds of formula I (process step (x) above)), and X¹, R¹, R², R³, R⁴ and R⁵ are as hereinbefore defined, with a compound of formula XVIII,

L⁶-T^{a}-Y^{a} XVIII

wherein T^{a} represents T and Y^{a} represents Y, provided that when Y represents —C(O)OR^{9b}, —S(O)₃R^{9c}, —P(O)(OR^{9d})₂, —P(O)(OR^{9e})N(R^{10f})R^{9f}, —P(O)(N(R^{10g})R^{9g})₂, —B(OR^{9h})₂ or —S(O)₂N(R¹⁰ⁱ)R⁹ⁱ, R^{9b} to R⁹ⁱ, R^{10f}, R^{10g} and R¹⁰ⁱ are other than H, and L⁶ represents a suitable leaving group known to those skilled in the art, such as halo (especially chloro or bromo), for example when Y^{a} represents —C(O)OR^{9b} or —S(O)₃R^{9c}, or C₁₋₃ alkoxy, for example when Y^{a} represents —B(OR^{9h})₂. The reaction may be performed under similar reaction conditions to those described hereinbefore in respect of process (x) above, followed by (if necessary) deprotection under standard conditions. The skilled person will appreciate that compounds of formula XVII in which L⁵ represents —B(OH)₂ are also compounds of formula I;
(xviii) for compounds of formula I in which T represents a single bond, Y represents —B(OR^{9h})₂ and R^{9h} represents H, reaction of a compound of formula XVII as hereinbefore defined with boronic acid or a protected derivative thereof (e.g. bis(pinacolato)diboron or triethyl borate), followed by (if necessary) deprotection under standard conditions;
(xix) for compounds of formula I in which T represents a single bond and Y represents —S(O)₃R^{9c}, reaction of a compound of formula XVII as hereinbefore defined with:
(A) for such compounds in which R^{9c} represents H, either SO₃ (or a suitable source of SO₃ such as a SO₃*pyridine or SO₃*Et₃N complex) or with SO₂ followed by treatment with N-chlorosuccinimide and then hydrolysis. Alternatively, a compound of formula XVII may be reacted with a protected sulfide, followed by deprotection and oxidation, or a compound of formula XVII may be reacted with chlorosulfonic acid (CIS(O)₂OH) followed by hydrolysis;
(B) for such compounds in which R^{9c} is other than H, chlorosulfonic acid followed by reaction with a compound of formula XXIII as defined hereinafter in which R^{9za} represents R^{9c},
all under standard conditions;
   (xx) for compounds of formula I in which T represents a single bond and Y represents

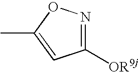

in which R^{9j} represents hydrogen, reaction of a corresponding compound of formula I in which T represents a C₂ alkylene group substituted at the carbon atom that is attached to the indole ring system by Z¹, in which Zⁱ represents ═O and Y represents —C(O)OR^{9b}, in which R^{9b} represents C₁₋₆ alkyl with hydroxylamine or an acid addition salt thereof, for example in the presence of base (e.g. NaOH), e.g. under similar reaction conditions to those described in inter alia J. Med. Chem. 43, 4930 (2000);
(xxi) for compounds of formula I in which T represents a single bond and Y represents

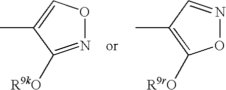

in which R^{9k} and R^{9r} represent hydrogen, reaction of a corresponding compound of formula I in which T represents a C₁ alkylene group substituted with G¹, in which G¹ represents -A¹-R^{11a}, A¹ represents —C(O)A²-, A² represents a single bond and R^{11a} represents H, and Y represents —C(O)OR^{9b}, in which R^{9b} represents methyl, or ethyl, respectively, with hydroxylamine or an acid addition salt thereof, for example in the presence of base (e.g. NaOH, or aniline, respectively) and an appropriate solvent (e.g. methanol, or water, respectively), e.g. under similar reaction conditions to those described in J. Med. Chem. 44, 1051 (2001), or inter alia J. Am. Chem. Soc., 58, 1152 (1936), respectively;
(xxii) for compounds of formula I in which T represents a single bond and Y represents

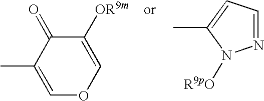

in which R^{9m} and R^{9p} represent hydrogen, reaction of a corresponding compound of formula I in which T represents a single bond, Y represents —B(OR^{9h})₂ and R^{9h} represents H with a compound of formula XVIII in which T^{a} represents a single bond, Y^{a} represents

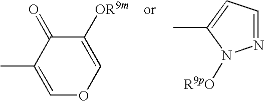

respectively, in which R^{9m} and R^{9p} represent hydrogen, and L⁶ preferably represents e.g. a halo group, such as Br, or I, respectively, or a protected derivative (e.g. at the OH group with, for example, a benzyl group) of either compound, for example under reaction conditions similar to those described hereinbefore in process (ii) above and/or in Heterocycles, 36, 1803 (1993), or in Bioorg. Med. Chem., 11, 1883 (2003), respectively, followed by (if necessary) deprotection under standard conditions;
(xxiii) for compounds of formula I in which T represents a single bond and Y represents

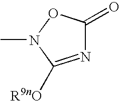

in which R⁹ⁿ represents hydrogen, reaction of a compound of formula XIX,

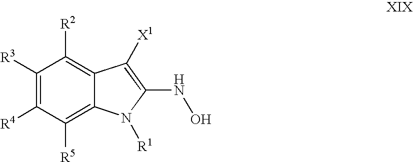

wherein X¹, R¹, R², R³, R⁴ and R⁵ are as hereinbefore defined with ethoxycarbonyl isocyanate in the presence of a suitable solvent (e.g. dichloromethane), followed by refluxing in the presence of Triton B and an alcoholic solvent (e.g. methanol), for example under similar reaction conditions to those described in J. Het. Chem., 19, 971 (1982);
(xxiv) for compounds of formula I in which T represents a single bond and Y represents

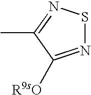

in which R^{9s} represents hydrogen, reaction of a compound of formula I in which T represents a single bond and Y represents —C(O)OR^{9b}, in which R^{9b} represents H with e.g. trimethylsilyl chloride (or the like), followed by reaction of the resultant intermediate with N₄S₄, for example under similar reaction conditions to those described in Heterocycles, 20, 2047 (1983);
(xxv) for compounds of formula I in which T represents a single bond and Y represents

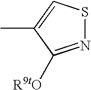

in which R^{9t} represents hydrogen, reaction of a compound of formula XX,

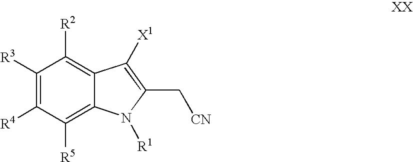

wherein X¹, R¹, R², R³, R⁴ and R⁵ are as hereinbefore defined with a base (e.g. NaH) and CS₂ in the presence of a suitable solvent (e.g. tetrahydrofuran), oxidation of the resultant intermediate in the presence of, for example, hydrogen peroxide, and finally heating the resultant intermediate in the presence of a strong acid, such as HCl, for example under similar reaction conditions to those described in inter alia Bioorg. Med. Chem. Lett., 2, 809 (1992);
(xxvi) for compounds of formula I in which T represents a single bond and Y represents

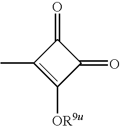

in which R^{9u} represents hydrogen, reaction of a corresponding compound of formula I in which T represents C₁ alkylene, Y represents —C(O)OR^{9b} and R^{9b} represents H or, preferably, an activated (e.g. acid halide) derivative thereof with 1,1,2,2-tetraethoxyethene, for example in the presence of base (e.g. triethylamine), followed by acid (e.g. aqueous HCl), e.g. under similar reaction conditions to those described in J. Am. Chem. Soc., 100, 8026 (1978);
(xxvii) for compounds of formula I in which T represents a single bond and Y represents

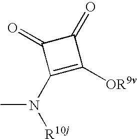

in which R^{9v} and R^{10j} represent H, reaction of a compound of formula XIX as hereinbefore defined with 3,4-dimethoxycyclobutene-1,2-dione, for example in the presence of base (e.g. KOH) and an appropriate solvent (e.g. methanol), followed by acid (e.g. aqueous HCl), e.g. under similar reaction conditions to those described in J. Org. Chem., 68, 9233 (2003);
(xxviii) for compounds of formula I in which T represents a single bond and Y represents

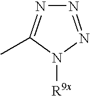

in which R^{9x} represents hydrogen, reaction of a compound of formula XXI,

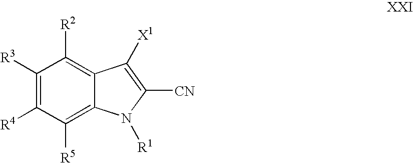

wherein X¹, R¹, R², R³, R⁴ and R⁵ are as hereinbefore defined with NaN₃ under standard conditions;
(xxix) for compounds of formula I in which T represents optionally substituted C₇₋₈ alkenylene or C₂₋₈ heteroalkylene (in which a point of unsaturation is between the carbon atoms that are É and é to the indole ring), reaction of a compound of formula XXII,

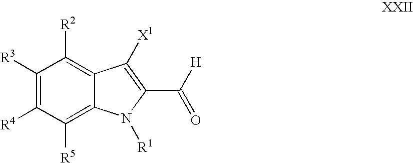

wherein X¹, R¹, R², R³, R⁴ and R⁵ are as hereinbefore defined with a compound of formula XXIIA,

(Ph)₃P═CH-T^{a}-Y XXIIA

or the like (e.g. the corresponding Horner-Wadsworth-Emmons reagent), wherein T^{a} represents a single bond or optionally substituted C₁₋₆ alkylene or C₂₋₆ heteroalkylene and Y is as hereinbefore defined, for example under standard Wittig reaction conditions, e.g. in the presence of a suitable organic solvent (e.g. DMF);
(xxx) for compounds of formula I in which T represents optionally substituted, saturated C₂₋₈ alkylene, saturated cycloalkylene, saturated C₂₋₈ heteroalkylene, saturated heterocycloalkylene, C₂₋₈ alkenylene, cycloalkenylene, C₂₋₈ heteroalkenylene or heterocycloalkenylene, reduction (e.g. hydrogenation) of a corresponding compound of formula I in which T represents optionally substituted C₂₋₈ alkenylene, cycloalkenylene, C₂₋₈ heteroalkenylene, heterocycloalkenylene, C₂₋₈ alkynylene, cycloalkynylene, C₂₋₈ heteroalkynylene or heterocycloalkynylene (as appropriate) under conditions that are known to those skilled in the art;
(xxxi) for compounds of formula I in which Y represents —C(O)OR^{9b}, —S(O)₃R^{9c}, —P(O)(OR^{9d})₂, or —B(OR^{9h})₂, in which R^{9b}, R^{9c}, R^{9d} and R^{9h} represent H, hydrolysis of a corresponding compound of formula I in which R^{9b}, R^{9c}, R^{9d} or R^{9h} (as appropriate) does not represent H, or, for compounds of formula I in which Y represents —P(O)(OR^{9d})₂ or S(O)₃R^{9c}, in which R^{9c} and R^{9d} represent H, a corresponding compound of formula I in which Y represents either —P(O)(OR^{9e})N(R^{10f})R^{9f}, —P(O)(N(R^{10g})R^{9g})₂ or —S(O)₂N(R¹⁰ⁱ)R⁹ⁱ (as appropriate), all under standard conditions;
(xxxii) for compounds of formula I in which Y represents —C(O)OR^{9b}, S(O)₃R^{9c}, —P(O)(OR^{9d})₂, —P(O)(OR^{9e})N(R^{10f})R^{9f} or —B(OR^{9h})₂ and R^{9b} to R^{9e} and R^{9h} (i.e. those R⁹ groups attached to an oxygen atom) do not represent H:
(A) esterification of a corresponding compound of formula I in which R^{9b} to R^{9e} and R^{9h} represent H; or
(B) trans-esterification of a corresponding compound of formula I in which R^{9b} to R^{9e} and R^{9h} do not represent H (and does not represent the same value of the corresponding R^{9b} to R^{9e} and R^{9h} group in the compound of formula I to be prepared),
   under standard conditions in the presence of the appropriate alcohol of formula XXIII,

R^{9za}OH XXIII

in which R^{9za} represents R^{9b} to R^{9e} or R^{9h} provided that it does not represent H;
(xxxiii) for compounds of formula I in which T represents a single bond, Y represents —C(O)OR^{9b} and R^{9b} is other than H, reaction of a compound of formula XXIIIA,

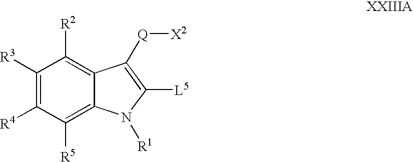

wherein L⁵, Q, X², R¹, R², R³, R⁴ and R⁵ are as hereinbefore defined, with a compound of formula XXIIIB,

L⁶C(O)OR^{9b1} XXIIIB

wherein R^{9b1} represents R^{9b} provided that it does not represent H, and L⁶ is as hereinbefore defined (e.g. L⁶ represents chloro or bromo), under conditions known to those skilled in the art;
(xxxiv) for compounds of formula I in which T represents a single bond, Y represents —C(O)OR^{9b} and R^{9b} is H, reaction of a compound of formula XXIIIA in which L⁵ represents either:
(I) an alkali metal (for example, such as one defined in respect of process step (xvii) above); or
(II) —Mg-halide,
   with carbon dioxide, followed by acidification under standard conditions known to those skilled in the art, for example, in the presence of aqueous hydrochloric acid;
   (xxxv) for compounds of formula I in which T represents a single bond and Y represents —C(O)OR^{9b}, reaction of a corresponding compound of formula XXIIIA in which L⁵ is a suitable leaving group known to those skilled in the art (such as a sulfonate group (e.g. a triflate) or, preferably, a halo (e.g. bromo or iodo) group), with CO (or a reagent that is a suitable source of CO (e.g. Mo(CO)₆ or CO₂(CO)₈)), in the presence of a compound of formula XXIIIC,

R^{9b}OH XXIIIC

wherein R^{9b} is as hereinbefore defined, and an appropriate catalyst system (e.g. a palladium catalyst such as one described hereinbefore in respect of process step (ii)) under conditions known to those skilled in the art;
(xxxvi) for compounds of formula I in which Y represents —C(O)OR^{9b} and R^{9b} represents H, hydrolysis of a corresponding compound of formula I in which R^{9b} does not represent H under standard conditions;
(xxxvii) for compounds of formula I in which Y represents —C(O)OR^{9b} and R^{9b} does not represent H:
(A) esterification of a corresponding compound of formula I in which R^{9b} represents H; or
(B) trans-esterification of a corresponding compound of formula I in which R^{9b} does not represent H (and does not represent the same value of R^{9b} as the compound of formula I to be prepared),
   under standard conditions in the presence of the appropriate alcohol of formula XXIIIC as hereinbefore defined but in which R^{9b} represents R^{9b1} as hereinbefore defined;
   (xxxviii) for compounds of formula I in which X¹ represents -Q-X² and Q represents —O—, reaction of a compound of formula XXIV,

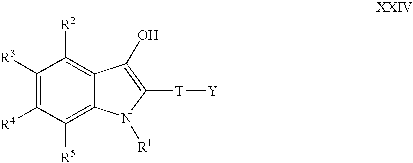

wherein R¹, R², R³, R⁴, R⁵, T and Y are as hereinbefore defined, with a compound of formula XXV,

X²L⁷ XXV

wherein L⁷ represents a suitable leaving group, such as a halo or sulfonate group, and X² is as hereinbefore defined, for example in the presence of a base or under reaction conditions such as those described hereinbefore in respect of process (ii) or process (xiii) above;
(xxxix) for compounds of formula I in which T represents a C₁ alkylene group substituted with G¹, in which G¹ represents -A¹-R^{11a}, A¹ represents —C(O)A²-, A² represents a single bond and R^{11a} represents H, and Y represents —C(O)OR^{9b}, in which R^{9b} is other than H, reaction of a corresponding compound of formula I in which the C_{I} alkylene group that T represents is unsubstituted with a C₁₋₆ alkyl (e.g. ethyl) formate in the presence of a suitable base (e.g. sodium ethoxide), for example under similar conditions to those described in Bioorg. Med. Chem. Lett., 13, 2709 (2003);
(xl) for compounds of formula I in which X¹ represents -Q-X², Q represents a single bond and X² represents C₁₋₈ alkyl or heterocycloalkyl substituted a to the indole ring by a G¹ substituent in which G¹ represents -A¹-R^{11a}, A¹ represents —OA⁵-, A⁵ represents a single bond and R^{11a} represents H, reaction of a corresponding compound of formula I in which X¹ represents H with a compound corresponding to a compound of formula VI, but in which X^{1b} represents -Q-X², Q represents a single bond and X² represents C₁₋₈ alkyl or heterocycloalkyl, both of which groups are substituted by a Z¹ group in which Z¹ represents ═O, under conditions known to those skilled in the art, for example optionally in the presence of an acid, such as a protic acid or an appropriate Lewis acid. Such substitutions are described in inter alia Bioorg. Med. Chem. Lett., 14, 4741-4745 (2004) and Tetrahedron Lett. 34, 1529 (1993);
(xli) for compounds of formula I in which X¹ represents -Q-X², Q represents a single bond and X² represents C₂₋₈ alkyl substituted (e.g. a to the indole ring) by a G¹ substituent in which G¹ represents -A¹-R^{11a}, A¹ represents —OA⁵-, A⁵ represents a single bond and R^{11a} represents H, reaction of a corresponding compound of formula I in which X² represents C₁₋₇ alkyl substituted (e.g. a to the indole ring) by a Z¹ group in which Z¹ represents ═O, with the corresponding Grignard reagent derivative of a compound of formula V in which L² represents chloro, bromo or iodo, Q^{a} is a single bond and X² represents C₁₋₇ alkyl, under conditions known to those skilled in the art;
(xlii) for compounds of formula I in which X¹ represents -Q-X², Q represents a single bond, and X² represents C₁₋₈ alkyl or heterocycloalkyl, both of which are unsubstituted in the position α to the indole ring, reduction of a corresponding compound of formula I in which X² represents C₁₋₈ alkyl substituted a to the indole ring by a G¹ substituent in which G¹ represents -A¹-R^{11a}, A¹ represents —OA⁵-, A⁵ represents a single bond and R^{11a} represents H, in the presence of a suitable reducing agent such as a mixture of triethyl silane and a protic acid (e.g. CF₃COOH) or a Lewis acid (e.g. (CH₃)₃SiOS(O)₂CF₃) for example under conditions described in inter alia Bioorg. Med. Chem. Lett., 14, 4741-4745 (2004);
(xliii) for compounds of formula I in which X¹ represents -Q-X², Q represents a single bond and X² represents C₁₋₈ alkyl or heterocycloalkyl, neither of which are substituted by Z¹ in which Z¹ represents ═O, reduction of a corresponding compound of formula I in which X² represents C₁₋₈ alkyl or heterocycloalkyl, which groups are substituted by one or more Z¹ groups in which Z¹ represents ═O under conditions known to those skilled in the art, for example employing NaBH₄ in the presence of an acid (e.g. CH₃COOH or CF₃COOH), Wolff-Kishner reduction conditions (i.e. by conversion of the carbonyl group to a hydrazone, followed by base induced elimination) or by conversion of the carbonyl to the thioacetal analogue (e.g. by reaction with a dithiane) followed by reduction with e.g. Raney nickel, all under reaction conditions known to those skilled in the art; or
(xliv) for compounds of formula I in which X¹ represents —N(R^{9a})-J-R^{10a}, reaction of a compound of formula XXIV as hereinbefore defined, with a compound of formula VI in which X^{1b} represents —N(R^{9a})-J-R^{10a} and R^{9a}, R^{10a} and J are as hereinbefore defined, for example under reaction conditions known to those skilled in the art (such as those described in Journal of Medicinal Chemistry 1996, Vol. 39, 4044 (e.g. in the presence of MgCl₂)).

Compounds of formula II may be prepared by:
(a) reaction of a compound of formula XXVI,

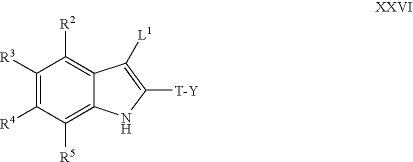

wherein L¹, R², R³, R⁴, R⁵, T and Y are as hereinbefore defined, with, for compounds of formula II in which X¹ represents:
(1) -Q-X² and Q represents a single bond or —C(O)—, a compound of formula V as hereinbefore defined; or
(2) —N(R^{9a})-J-R^{10a} or -Q-X², in which Q represents —O— or —S—, a compound of formula VI as hereinbefore defined,
for example under reaction conditions similar to those described hereinbefore in respect of preparation of compounds of formula I (processes (ii) and (iv), respectively) above;
(b) for compounds of formula II in which X¹ represents -Q-X² and Q represents —C(O)—, reaction of a corresponding compound of formula II in which X¹ represents H, with a compound of formula V in which Q^{a} represents —C(O)— and L² represents a suitable leaving group, for example under conditions such as those described in respect of preparation of compounds of formula I (process (iii)) above;
(c) for compounds of formula II in which X¹ represents -Q-X² and Q represents —S—, reaction of a corresponding compound of formula II in which X¹ represents H with a compound of formula VI in which X^{1b} represents -Q-X² and Q represents —S—, for example under conditions such as those described hereinbefore in respect of preparation of compounds of formula I (process (v)) above;
(d) for compounds of formula II in which Q represents —S(O)— or —S(O)₂—, oxidation a corresponding compound of formula II in which Q represent —S—;
(e) for compounds of formula II in which X¹ represents -Q-X², X² represents C₁₋₈ alkyl substituted by G¹, G¹ represents A¹-R^{11a}, A¹ represents —N(R^{12a})A⁴- and A⁴ is a single bond (provided that Q represents a single bond when X² represents substituted C₁ alkyl), reaction of a compound of formula XXVII,

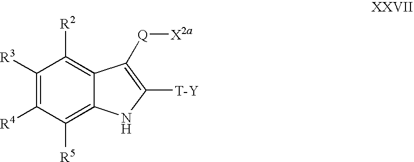

wherein Q, X^{2a}, R², R³, R⁴, R⁵, T and Y are as hereinbefore defined by reductive amination in the presence of a compound of formula VIII as hereinbefore defined;
(ea) for compounds of formula II in which X¹ represents -Q-X², Q represents a single bond, X² represents methyl substituted by G¹, G¹ represents -A¹-R^{11a}, A¹ represents —N(R^{12a})A⁴ is a single bond and R^{11a} and R^{12a} are preferably methyl, reaction of a corresponding compound of formula II in which X¹ represents H, with a mixture of formaldehyde (or equivalent reagent) and a compound of formula VIII as hereinbefore defined, for example under reaction conditions similar to those described hereinbefore in respect of preparation of compounds of formula I (process (viia)) above;
(f) for compounds of formula II in which X¹ represents -Q-X², Q represents a single bond and X² represents optionally substituted C₂₋₈ alkenyl (in which a point of unsaturation is between the carbon atoms that are É and é to the indole ring), reaction of a compound of formula XXVI in which L¹ represents halo (e.g. iodo) with a compound of formula XXVII as hereinbefore defined, or reaction of compound of formula XXIV in which Q represents a single bond and X^{2a} represents —CHO with a compound of formula IXB or a compound of formula IXC as hereinbefore defined, for example under reaction conditions similar to those described hereinbefore in respect of preparation of compounds of formula I (process (viii)) above;
(g) for compounds of formula II in which X¹ represents -Q-X² and X² represents optionally substituted, saturated C₂₋₈ alkyl, saturated cycloalkyl, saturated heterocycloalkyl, C₂₋₄ alkenyl, cycloalkenyl or heterocycloalkenyl, reduction (e.g. hydrogenation) of a corresponding compound of formula II in which X² represents optionally substituted C₂₋₈ alkenyl, cycloalkenyl, heterocycloalkenyl, C₂₋₈ alkynyl, cycloalkynyl or heterocycloalkynyl (as appropriate);
(h) for compounds of formula II in which D represents a single bond, —C(O)—, —C(R⁷)(R⁸)—, C₂₋₈ alkylene or —S(O)₂—, reaction of a compound of formula XXVIII,

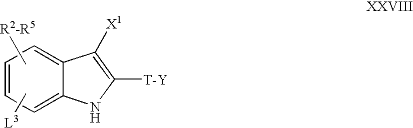

wherein X¹, L³, R²-R⁵, T and Y are as hereinbefore defined with a compound of formula XI as hereinbefore defined, for example under reaction conditions similar to those described hereinbefore in respect of preparation of compounds of formula I (process (x)) above;
(i) for compounds of formula II in which D represents —S—, —O— or C₂₋₄ alkynylene in which the triple bond is adjacent to E, reaction of a compound of formula XXVIII as hereinbefore defined in which L³ represents L² as hereinbefore defined (for example —B(OH)₂) with a compound of formula XII as hereinbefore defined, for example under reaction conditions similar to those described hereinbefore in respect of preparation of compounds of formula I (process (xi)) above;
(j) for compounds of formula II in which D represents —S(O)— or —S(O)₂—, oxidation of a corresponding compound of formula II in which D represents —S—;
(k) for compounds of formula II in which D represents —O— or —S—, reaction of a compound of formula XXIX,

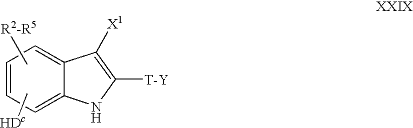

wherein X¹, R²-R⁵, T and Y are as hereinbefore defined, with a compound of formula XIV as hereinbefore defined;
(l) for compounds of formula II in which X¹ represents —N(R^{9a})-J-R^{10a}, reaction of a compound of formula XXX,

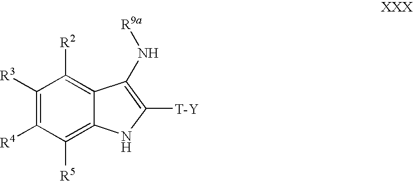

wherein R², R³, R⁴, R⁵, R^{9a}, T and Y are as hereinbefore defined with a compound of formula XVI as hereinbefore defined, for example under reaction conditions similar to those described hereinbefore in respect of preparation of compounds of formula I (process (xiv)) above;
(m) for compounds of formula II in which X¹ represents —N(R^{9a})-J-R^{10a}, J represents a single bond and R^{10a} represents a C₁₋₈ alkyl group, reduction of a corresponding compound of formula II, in which J represents —C(O)— and R^{10a} represents H or a C₁₋₇ alkyl group, for example under reaction conditions similar to those described hereinbefore in respect of preparation of compounds of formula I (process (xv)) above;
(n) for compounds of formula II in which X¹ represents halo, reaction of a compound of formula II wherein X¹ represents H, with a reagent or mixture of reagents known to be a source of halide atoms, for example under reaction conditions similar to those described hereinbefore in respect of preparation of compounds of formula I (process (xvi)) above;
(o) for compounds of formula II in which T and Y are as hereinbefore defined, provided that when Y represents —C(O)OR^{9b}, —S(O)₃R^{9c}, —P(O)(OR^{9d})₂, —P(O)(OR^{9e})N(R^{10f})R^{9f}, —P(O)(N(R^{10g})R^{9g})₂, —B(OR^{9h})₂ or —S(O)₂N(R¹⁰ⁱ)R⁹ⁱ, R^{9b} to R⁹ⁱ, R^{10f}; R^{10g} and R¹⁰ⁱ are other than H, reaction of a compound of formula XXXI,

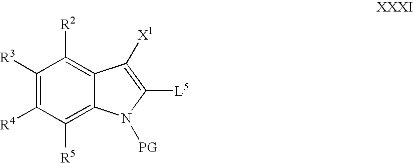

wherein PG represents a suitable protecting group, such as —S(O)₂Ph, —C(O)O⁻, —C(O)OtBu or —C(O)N(Et)₂) and L⁵, X¹, R², R³, R⁴ and R⁵ are as hereinbefore defined, with a compound of formula XVIII as hereinbefore defined, or a protected derivative thereof, for example under similar coupling conditions to those described hereinbefore in respect of process (xvii) above, followed by deprotection of the resultant compound under standard conditions;
(p) for compounds of formula II in which T represents a single bond, Y represents —B(OR^{9h})₂ and R^{9h} represents H, reaction of a compound of formula XXXI as hereinbefore defined with boronic acid or a protected derivative thereof (e.g. bis(pinacolato)diboron or triethyl borate), followed by deprotection of the resultant compound under standard conditions;
(q) for compounds of formula II in which T represents a single bond and Y represents —S(O)₃R^{9c}, reaction of a compound of formula XXXI as hereinbefore defined with:
   (A) for such compounds in which R^{9c} represents H, either SO₃ or with SO₂ followed by treatment with N-chlorosuccinimide and then hydrolysis;
   (B) for such compounds in which R^{9c} is other than H, chlorosulfonic acid followed by reaction with a compound of formula XXIII as defined hereinbefore in which R^{9za} represents R^{9c}, all under standard conditions such as those described hereinbefore in respect of preparation of compounds of formula I (process (xix)) above;
(r) for compounds of formula II in which T represents a single bond and Y represents

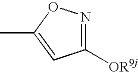

in which R^{9j} represents hydrogen, reaction of a corresponding compound of formula II in which T represents a C₂ alkylene group substituted at the carbon atom that is attached to the indole ring system by Z¹, in which Z¹ represents ═O and Y represents —C(O)OR^{9b}, in which R^{9b} represents C₁₋₆ alkyl with hydroxylamine or an acid addition salt thereof, for example under reaction conditions similar to those described hereinbefore in respect of preparation of compounds of formula I (process (xx)) above;
(s) for compounds of formula II in which T represents a single bond and Y represents

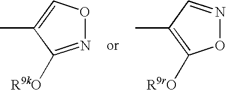

in which R^{9k} and R^{9r} represent hydrogen, reaction of a corresponding compound of formula II in which T represents a C₁ alkylene group substituted with G¹, in which G¹ represents -A¹-R^{11a}, A¹ represents —C(O)A²-, A² represents a single bond and R^{11a} represents H, and Y represents —C(O)OR^{9b}, in which R^{9b} represents methyl, or ethyl, respectively, with hydroxylamine or an acid addition salt thereof, for example under reaction conditions similar to those described hereinbefore in respect of preparation of compounds of formula I (process (xxi)) above;
(t) for compounds of formula II in which T represents a single bond and Y represents

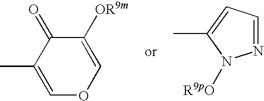

in which R^{9m} and R^{9p} represent hydrogen, reaction of a corresponding compound of formula II in which T represents a single bond, Y represents —B(OR^{9h})₂ and R^{9h} represents H with a compound of formula XVIII in which T^{a} represents a single bond, Y^{a} represents

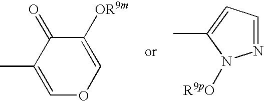

respectively, in which R^{9m} and R^{9p} represent hydrogen, and L⁶ preferably represents e.g. a halo group, such as Br, or I, respectively, or a protected derivative (e.g. at the OH group with, for example, a benzyl group) of either compound, for example under reaction conditions similar to those described hereinbefore in respect of preparation of compounds of formula I (process (xxii)) above;
(u) for compounds of formula II in which T represents a single bond and Y represents

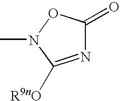

in which R⁹ⁿ represents hydrogen, reaction of a compound of formula XXXII,

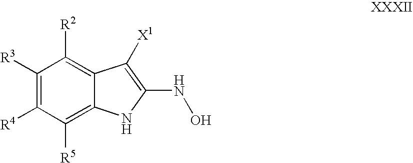

wherein X¹, R¹, R², R³, R⁴ and R⁵ are as hereinbefore defined with ethoxycarbonyl isocyanate, for example under reaction conditions similar to those described hereinbefore in respect of preparation of compounds of formula I (process (xxiii)) above;
(v) for compounds of formula II in which T represents a single bond and Y represents

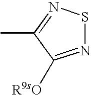

in which R^{9s} represents hydrogen, reaction of a compound of formula II in which T represents a single bond and Y represents —C(O)OR^{9b}, in which R^{9b} represents H with e.g. trimethylsilyl chloride (or the like), followed by reaction of the resultant intermediate with N₄S₄, for example under reaction conditions similar to those described hereinbefore in respect of preparation of compounds of formula I (process (xxiv)) above;
(w) for compounds of formula II in which T represents a single bond and Y represents

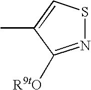

in which R^{9t} represents hydrogen, reaction of a compound of formula XXXIII,

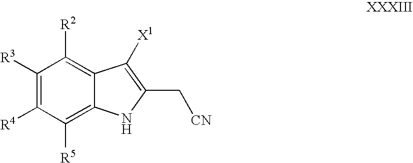

wherein X¹, R², R³, R⁴ and R⁵ are as hereinbefore defined with a base (e.g. NaH) and CS₂ the presence of a suitable solvent (e.g. tetrahydrofuran), oxidation of the resultant intermediate in the presence of, for example, hydrogen peroxide, and finally heating the resultant intermediate in the presence of a strong acid, such as HCl, for example under reaction conditions similar to those described hereinbefore in respect of preparation of compounds of formula I (process (xxv)) above;
(x) for compounds of formula I in which T represents a single bond and Y represents

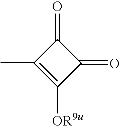

in which R^{9u} represents hydrogen, reaction of a corresponding compound of formula II in which T represents C₁ alkylene, Y represents —C(O)OR^{9b} and R^{9b} represents H or, preferably, an activated (e.g. acid halide) derivative thereof with 1,1,2,2-tetraethoxyethene, for example under reaction conditions similar to those described hereinbefore in respect of preparation of compounds, of formula I (process (xxvi)) above;
(y) for compounds of formula II in which T represents a single bond and Y represents

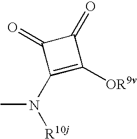

in which R^{9v} and R^{10j} independently represent hydrogen, reaction of a compound of formula XXXII as hereinbefore defined with 3,4-dimethoxycyclobutene-1,2-dione, for example under reaction conditions similar to those described hereinbefore in respect of preparation of compounds of formula I (process (xxvii)) above;
(z) for compounds of formula II in which T represents a single bond and Y represents

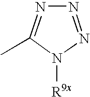

in which R^{9x} represents hydrogen, reaction of a compound of formula XXXIV,

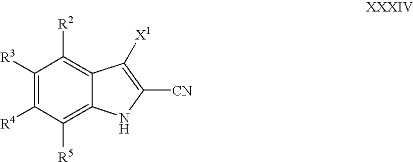

wherein X¹, R², R³, R⁴ and R⁵ are as hereinbefore defined with NaN₃ under standard conditions;
(aa) for compounds of formula II in which T represents optionally substituted C₂₋₈ alkenylene or C₂₋₈ heteroalkylene (in which a point of unsaturation is between the carbon atoms that are É and é to the indole ring), may be prepared by reaction of a corresponding compound of formula XXXV,

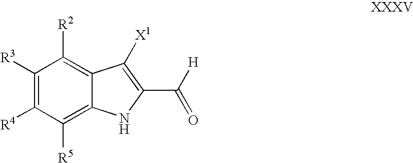

wherein X¹, R², R³, R⁴ and R⁵ are as hereinbefore defined with a compound of formula XXIIA as hereinbefore defined, under standard Wittig reaction conditions;
(ab) for compounds of formula II in which T represents optionally substituted, saturated C₂₋₈ alkylene, saturated cycloalkylene, saturated C₂₋₈ heteroalkylene, saturated heterocycloalkylene, C₂₋₈ alkenylene, cycloalkenylene, C₂₋₈ heteroalkenylene or heterocycloalkenylene, reduction (e.g. hydrogenation) of a corresponding compound of formula II in which T represents optionally substituted C₂₋₈ alkenylene, cycloalkenylene, C₂₋₈ heteroalkenylene, heterocycloalkenylene, C₂₋₈ alkynylene, cycloalkynylene, C₂₋₈ heteroalkynylene or heterocycloalkynylene (as appropriate);
(ac) for compounds of formula II in which Y represents —C(O)OR^{9b}, —S(O)₃R^{9c}, —P(O)(OR^{9d})₂, or —B(OR^{9h})₂, in which R^{9b}, R^{9c}, R^{9d} and R^{9h} represent H, hydrolysis of a corresponding compound of formula II in which R^{9b}, R^{9c}, R^{9d} or R^{9h} (as appropriate) does not represent H, or, for compounds of formula II in which Y represents P(O)(OR^{9d})₂ or S(O)₃R^{9c}, in which R^{9c} and R^{9d} represent H, a corresponding compound of formula II in which Y represents either —P(O)(OR^{9e})N(R^{10f})R^{9f}, —P(O)(N(R^{10g})R^{9g})₂ or —S(O)₂N(R¹⁰ⁱ)R⁹ⁱ (as appropriate);
(ad) for compounds of formula II in which Y represents —C(O)OR^{9b}, —S(O)₃R^{9c}, —P(O)(OR^{9d})₂, —P(O)(OR^{9e})N(R^{10f})R^{9f} or —B(OR^{9h})₂ and R^{9b} to R^{9e} and R^{9h} (i.e. those R⁹ groups attached to an oxygen atom), do not represent H:
   (A) esterification of a corresponding compound of formula II in which R^{9b} to R^{9e} and R^{9h} represents H; or
   (B) trans-esterification of a corresponding compound of formula II in which R^{9b} to R^{9e} and R^{9h} do not represent H (and does not represent the same value of the corresponding R^{9b} to R^{9e} and R^{9h} group in the compound of formula II to be prepared),
under standard conditions in the presence of the appropriate alcohol of formula XXIII as hereinbefore defined;
(ae) for compounds of formula II in which T represents a single bond, Y represents —C(O)OR^{9b} and R^{9b} is other than H, reaction of a compound of formula XXXVA,

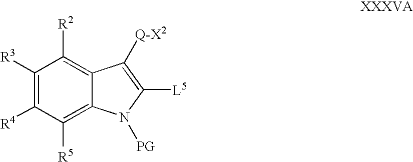

wherein PG represents a suitable protecting group, such as —S(O)₂Ph, —C(O)O⁻, —C(O)OtBu or —C(O)N(Et)₂) and L⁵, Q, X², R², R³, R⁴ and R⁵ are as hereinbefore defined, with a compound of formula XXIIIB as hereinbefore defined, for example under reaction conditions similar to those described hereinbefore in respect of preparation of compounds of formula I (process (xxxiii)) above), followed by deprotection of the resultant compound under standard conditions;
(af) for compounds of formula II in which T represents a single bond, Y represents —C(O)OR^{9b} and R^{9b} is H, reaction of a compound of formula XXXVA in which L⁵ represents an alkali metal, or —Mg-halide, with carbon dioxide, followed by acidification;
(ag) for compounds of formula II in which T represents a single bond, Y represents —C(O)OR^{9b}, reaction of a corresponding compound of formula XXXVA in which L⁵ represents a suitable leaving group known to those skilled in the art (such as a halo (e.g. bromo or iodo) group) with CO (or a suitable reagent that is a source of CO), in the presence of a compound of formula XXIIIC as hereinbefore defined;
(ah) for compounds of formula II in which Y represents —C(O)OR^{9b} and R^{9b} represents H, hydrolysis of a corresponding compound of formula II in which R^{9b} does not represent H;
(ai) for compounds of formula II in which Y represents —C(O)OR^{9b} and R^{9b} does not represent H:
   (A) esterification of a corresponding compound of formula II in which R^{9b} represents H; or
   (B) trans-esterification of a corresponding compound of formula II in which R^{9b} does not represent H (and does not represent the same value of R^{9b} as the compound of formula II to be prepared);
(aj) for compounds of formula II in which X¹ represents -Q-X² and Q represents —O—, reaction of a compound of formula XXXVI,

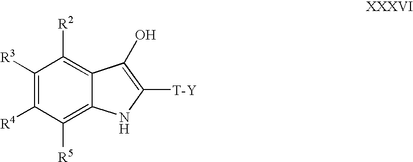

wherein R², R³, R⁴, R⁵, T and Y are as hereinbefore defined, with a compound of formula XXV as hereinbefore defined;
(ak) for compounds of formula II in which T represents a C₁ alkylene group substituted with G¹, in which G¹ represents -A¹-R^{11a}, A¹ represents —C(O)A²-, A² represents a single bond and R^{11a} represents H, and Y represents —C(O)OR^{9b}, in which R^{9b} is other than H, reaction of a corresponding compound of formula II in which the C₁ alkylene group that T represents is unsubstituted with a C₁₋₆ alkyl formate in the presence of a suitable base;
(al) for compounds of formula II in which X¹ represents -Q-X², Q represents a single bond and X² represents C₁₋₈ alkyl or heterocycloalkyl substituted a to the indole ring by a G¹ substituent in which G¹ represents -A¹-R^{11a}, A¹ represents —OA⁵-, A⁵ represents a single bond and R^{11a} represents H, reaction of a corresponding compound of formula II in which X¹ represents H with a compound corresponding to a compound of formula VI, but in which X^{1b} represents -Q-X², Q represents a single bond and X² represents C₁₋₈ alkyl or heterocycloalkyl, both of which groups are substituted by a Z¹ group in which Z¹ represents ═O, for example under reaction conditions similar to those described hereinbefore in respect of preparation of compounds of formula I (process (xl)) above;
(am) for compounds of formula II in which X¹ represents -Q-X², Q represents a single bond and X² represents C₂₋₈ alkyl substituted (e.g. a to the indole ring) by a G¹ substituent in which G¹ represents -A¹-R^{11a}, A¹ represents —OA⁵-, A⁵ represents a single bond and R^{11a} represents H, reaction of a corresponding compound of formula II in which X² represents C₁₋₇ alkyl substituted (e.g. α to the indole ring) by a Z¹ group in which Z¹ represents ═O, with the corresponding Grignard reagent derivative of a compound of formula V in which L² represents chloro, bromo or iodo, Q^{a} is a single bond and X² represents C₁₋₇ alkyl, under conditions known to those skilled in the art;
(an) for compounds of formula II in which X¹ represents -Q-X², Q represents a single bond, and X² represents C₁₋₈ alkyl or heterocycloalkyl, both of which are unsubstituted in the position α to the indole ring, reduction of a corresponding compound of formula II in which X² represents C₁₋₈ alkyl substituted a to the indole ring by a G¹ substituent in which G¹ represents -A¹-R^{11a}, A¹ represents —OA⁵-, A⁵ represents a single bond and R^{11a} represents H, for example under reaction conditions similar to those described hereinbefore in respect of preparation of compounds of formula I (process (xlii)) above;
(ao) for compounds of formula II in which X¹ represents -Q-X², Q represents a single bond and X² represents C₁₋₈ alkyl or heterocycloalkyl, neither of which are substituted by Z¹ in which Z¹ represents ═O, reduction of a corresponding compound of formula II in which X² represents C₁₋₈ alkyl or heterocycloalkyl, which groups are substituted by one or more Z¹ groups in which Z¹ represents ═O, for example under reaction conditions similar to those described hereinbefore in respect of preparation of compounds of formula I (process (xliii)) above; or
(ap) for compounds of formula II in which X¹ represents —N(R^{9a})-J-R^{10a}, reaction of a compound of formula XXXVI as hereinbefore defined, with a compound of formula VI in which X^{1b} represents —N(R^{9a})-J-R^{10a} and R^{9a}, R^{10a} and J are as hereinbefore defined, for example under conditions similar to those described hereinbefore in respect of preparation of compounds of formula I (process (xliv)) above.

Compounds of formula IV may be prepared as follows:
(a) Reaction of a compound of formula XXVI as hereinbefore defined with a compound of formula XXXVII,

R¹L² XXXVII
wherein R¹ and L² are as hereinbefore defined or a compound of formula III as hereinbefore defined, for example under reaction conditions similar to those described hereinbefore in respect of preparation of compounds of formula I (processes (ii) and (i), respectively) above; or
(b) for compounds of formula IV in which L¹ represents a sulfonate group, reaction of a compound of formula XXIV as hereinbefore defined, with an appropriate reagent for the conversion of the hydroxyl group to the sulfonate group (e.g. tosyl chloride, mesyl chloride, triflic anhydride and the like) under conditions known to those skilled in the art.

Compounds of formula VII may be prepared by:
(a) for compounds of formula VII in which D represents a single bond, —C(O)—, —C(R⁷)(R⁸)—, C₂₋₄ alkylene or —S(O)₂—, reaction of a compound of formula XXXVIII,

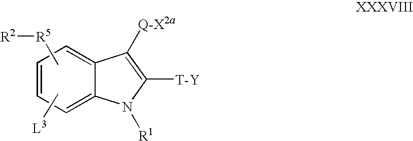

wherein Q, X^{2a}, L³, R¹, R²-R⁵, T and Y are as hereinbefore defined (L³ in particular may represent halo, such as bromo) with a compound of formula XI as hereinbefore defined (in which L⁴ may in particular represent —B(OH₂)), for example under reaction conditions similar to those described hereinbefore in respect of preparation of compounds of formula I (process (x)) above;
(b) reaction of a compound of formula XXVII as hereinbefore defined with a compound of formula III as hereinbefore defined, for example under reaction conditions similar to those described hereinbefore in respect of preparation of compounds of formula I (process (i)) above); or
(c) for compounds of formula VII in which Q represents a single bond and X^{2a} represents —CHO, reaction of a corresponding compound of formula I in which X¹ represents H with a mixture of DMF and, for example, oxalyl chloride, phosgene or P(O)Cl₃ (or the like) in an appropriate solvent system (e.g. DMF or dichloromethane).

Compounds of formula X may be prepared by reaction of a compound of formula XXVIII as hereinbefore defined, with a compound of formula III as hereinbefore defined, for example under reaction conditions similar to those described hereinbefore in respect of preparation of compounds of formula I (process (i)) above.

Compounds of formula X in which L³ represents L² may be prepared by reaction of a compound of formula X in which L³ represents L¹, with an appropriate reagent for the conversion of the L¹ group to the L² group. This conversion may be performed by methods known to those skilled in the art, for example, compounds of formula X, in which L³ is 4,4,5,5-tetramethyl-1,3,2-dioxaborolan-2-yl may be prepared by reaction of the reagent bis(pinacolato)diboron with a compound of formula X in which L³ represents L¹, for example under reaction conditions similar to those described hereinbefore in respect of preparation of compounds of formula I (process (ii)) above).

Compounds of formulae XV and XXX may be prepared by reaction of a corresponding compound of formula IV, or XXVI, respectively, with a compound of formula XXXIX,

R^{9a}NH₂ XXXIX

wherein R^{9a} is as hereinbefore defined, for example under reaction conditions similar to those described hereinbefore in respect of preparation of compounds of formula I (process (ii)) above).

Compounds of formulae XVII and XXXI in which L⁵ represents an appropriate alkali metal, such as lithium may be prepared by reaction of a compound of formula XL,

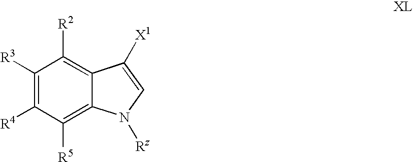

wherein R^{z} represents R¹ (in the case of a compound of formula XVII) or PG (in the case of a compound of formula XXXI), and PG, X¹, R¹, R², R³, R⁴ and R⁵ are as hereinbefore defined, with an appropriate base, such lithium diisopropylamide or BuLi under standard conditions. Compounds of formulae XVII and XXXI in which L⁵ represents —Mg-halide may be prepared from a corresponding compound of formula XVII or XXXI (as appropriate) in which L⁵ represents halo, for example under conditions such as those described hereinbefore in respect of process step (x). Compounds of formulae XVII and XXXI in which L⁵ represents, for example, a zinc-based group, or a halo or boronic acid group a group (such as a zinc-based group, halo or a boronic acid) may be prepared by reacting a corresponding compound of formula XVII or XXXI in which L⁵ represents an alkali metal with an appropriate reagent for introduction of the relevant group, for example by a metal exchange reaction (e.g. a Zn transmetallation), by reaction with a suitable reagent for the introduction of a halo group (for example, a reagent described hereinbefore in respect of preparation of compounds of formula I (process (xvi)) or, for the introduction of a boronic acid group, reaction with, for example, boronic acid or a protected derivative thereof (e.g. bis(pinacolato)diboron or triethyl borate) followed by (if necessary) deprotection under standard conditions.

Compounds of formula XVII in which L⁵ represents halo may alternatively by prepared by reaction of a compound of formula XLI,

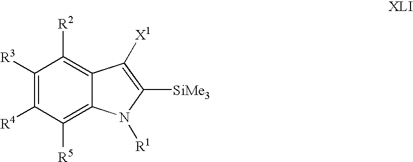

wherein R¹, R², R³, R⁴ and R⁵ are as hereinbefore defined, with an appropriate reagent known to be a suitable source of halide atoms (see for example process (xvi) above in respect of preparation of compounds of formula I).

Compounds of formulae XX and XXXIII, and XXII and XXXV, may be prepared by reduction of a corresponding compound of formula I, or of formula II, respectively, in which T represents a single bond and Y represents —C(O)OR^{9b}, to the corresponding primary alcohol (using e.g. LiAlH₄), followed by reaction of the relevant resultant intermediate with, in the case of preparation of a compound of formula XX or XXXIII, SOCl₂, MeSO₂Cl or bromine followed by a suitable source of cyanide ions (e.g. NaCN or KCN) or, in the case of preparation of a compound of formula XXII or XXXV, oxidation to the aldehyde in the presence of a suitable oxidising agent, such as MnO₂, in all cases under reaction conditions that will be well known to those skilled in the art. In the case of the latter, the skilled person will appreciate that an appropriate reagent for the reduction of the ester group directly to the aldehyde may be employed (e.g. DIBAL).

Compounds of formulae XXI and XXXIV may be prepared by conversion of a corresponding compound of formula I which T represents a single bond and Y represents —C(O)OR^{9b} to the corresponding primary amide (e.g. when R^{9b} is H, by reaction with SOCl₂ followed by ammonia or when R^{9b} is other than H, by reaction with ammonia), followed by dehydration of the resultant intermediate in the presence of a suitable dehydrating agent, such as POCl₃, in all cases under reaction conditions that will be well known to those skilled in the art.

Compounds of formula XXVI may be prepared by standard techniques. For example compounds of formula XXVI in which D represents a single bond, —C(O)—, —C(R⁷)(R⁸)—, C₂₋₄ alkylene or —S(O)₂—, may be prepared by reaction of a compound of formula XLII,

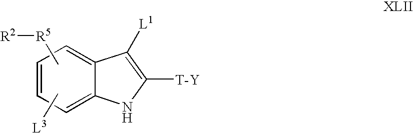

wherein L¹, L³, R²-R⁵ T and Y are as hereinbefore defined with a compound of formula XI as hereinbefore defined, for example under reaction conditions similar to those described hereinbefore in respect of preparation of compounds of formula I (process (x)) above.

Compounds of formulae XXVII and XXXVIII, in which Q represents a single bond and X^{2a} represents —CHO, may be prepared from compounds of formulae II, or X, respectively, in which X¹ represents H, by reaction with a mixture of DMF and, for example, oxalyl chloride, phosgene or P(O)Cl₃ (or the like) in an appropriate solvent system (e.g. DMF or dichloromethane) for example as described hereinbefore.

Compounds of formulae III, V, VI, VIII, IXA, IXB, IXC, XI, XII, XIII, XIV, XVI, XVIII, XIX, XXIIA, XXIII, XXIIIA, XXIIIB, XXIIIC, XXIV, XXV, XXVIII, XXIX, XXXII, XXXVA, XXXVI, XXXVII, XXXIX, XL, XLI and XLII are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991.

Indoles of formulae II, IV, VII, X, XIII, XV, XVII, XIX, XX, XXI, XXII, XXIIIA, XXIV, XXVI, XXVII, XXVIII, XXIX, XXX, XXXI, XXXII, XXXIII, XXXIV, XXXV, XXXVA, XXXVI, XXXVIII, XL, XLI and XLII may also be prepared with reference to a standard heterocyclic chemistry textbook (e.g. “Heterocyclic Chemistry” by J. A. Joule, K. Mills and G. F. Smith, 3^{rd} edition, published by Chapman & Hall or “Comprehensive Heterocyclic Chemistry II” by A. R. Katritzky, C. W. Rees and E. F. V. Scriven, Pergamon Press, 1996) and/or made according to the following general procedures.

For example, compounds of formulae II, XXVIII and XXIX in which X¹ represents H, —N(R^{9a})-J-R^{10a} or -Q-X², may be prepared by reaction of a compound of formula XLIII,

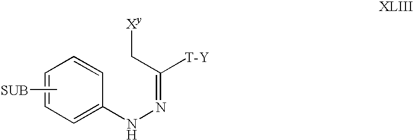

wherein SUB represents the substitution pattern that is present in the relevant compound to be formed (in this case, the compound of formula II, XXVIII or XXIX, respectively), X^{y} represents H, —N(R^{9a})-J-R^{10a} or -Q-X², and R^{9a}, R^{10a}, J, Q, X², T and Y are as hereinbefore defined, under Fischer indole synthesis conditions known to the person skilled in the art.

Compounds of formulae II, XXVIII and XXIX in which X¹ represents H may be prepared by reaction of a compound of formula XLIV,

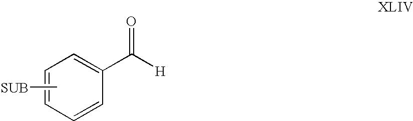

wherein SUB is as hereinbefore defined with a compound of formula XLV,

N₃CH₂-T-Y XLV

wherein T is as hereinbefore defined and preferably a single bond or optionally substituted arylene or heteroarylene, and Y is as hereinbefore defined and, when T represents a single bond, preferably represents —C(O)OR^{9b} in which R^{9b} preferably does not represent hydrogen, under conditions known to the person skilled in the art (i.e. conditions to induce a condensation reaction, followed by a thermally induced cyclisation).

Compounds of formulae XXIV and XXXVI may be prepared by reaction of a compound of formula XLVI,

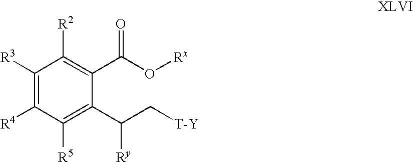

wherein R^{x} represents a C₁₋₆ alkyl group, R^{y} represents either R¹ (as required for the formation of compounds of formula XXIV), hydrogen (as required for the formation of compounds of formula XXXVI) or a nitrogen-protected derivative thereof, and R¹, R², R³, R⁴, R⁵, T and Y are as hereinbefore defined for example under cyclisation conditions known to those skilled in the art.

Compounds of formulae II and XXIX wherein X¹ represents —NH₂, may be prepared by reaction of a compound of formula XLVII,

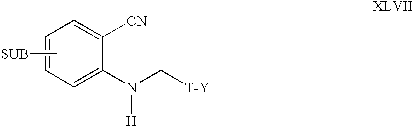

wherein SUB, T and Y are as hereinbefore defined, for example under intramolecular cyclisation conditions known to those skilled in the art.

Compounds of formulae II and XXIX in which X¹ represents H, —N(R^{9a})-J-R^{10a} or -Q-X² in which Q represents a single bond or —C(O)—; may alternatively be prepared by reaction of a compound of formula XLVIII,

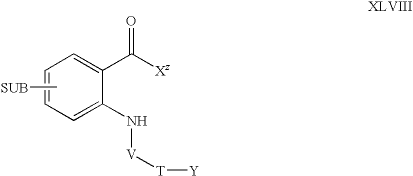

wherein V represents either —C(O)— or —CH₂—, X^{z} represents H, —N(R^{9a})-J-R^{10a} or -Q-X² in which Q represents a single bond or —C(O)— and SUB, R^{9a}, R^{10a}, J, T and Y are as hereinbefore defined. When V represents —C(O)—, the intramolecular cyclisation may be induced by a reducing agent such as TiCl₃/C₈K, TiCl₄/Zn or SmI₂ under conditions known to the skilled person, for example, at room temperature in the presence of a polar aprotic solvent (such as THF). When V represents —CH₂—, the reaction may be performed in the presence of base under intramolecular condensation reaction conditions known to the skilled person.

Compounds of formula XLIII may be prepared by:
(a) reaction of a compound of formula XLIX,

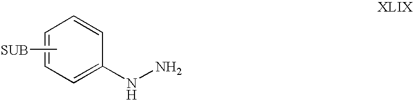

wherein SUB is as hereinbefore defined with a compound of formula L,

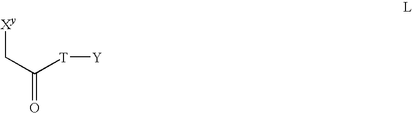

wherein X^{y}, T and Y are as hereinbefore defined under condensation conditions known to the skilled person;
(b) reaction of a compound of formula LI,

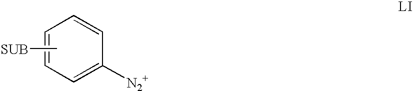

wherein SUB is as hereinbefore defined with a compound of formula LII,

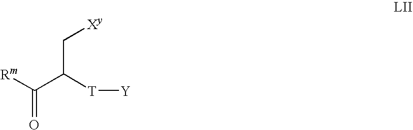

wherein R^{m} represents OH, O—C₁₋₆ alkyl or C₁₋₆ alkyl and X^{y}, T and

Y are as hereinbefore defined, for example under Japp-Klingemann conditions known to the skilled person.

Compounds of formula XLVIII may be prepared by reaction of a compound of LIII,

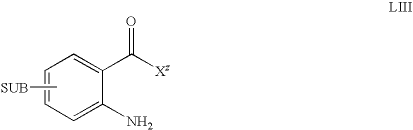

wherein SUB and X^{z} are as hereinbefore defined with a compound of formula LIV,

Y-T-V—Cl LIV

wherein T, Y and V are as hereinbefore defined, under standard coupling conditions.

Compounds of formulae XLIV, XLV, XLVI, XLVII, XLIX, L, LI, LII, LIII and LIV are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991.

The substituents X¹, R¹, R², R³, R⁴, R⁵, T and Y in final compounds of the invention or relevant intermediates may be modified one or more times, after or during the processes described above by way of methods that are well known to those skilled in the art. Examples of such methods include substitutions, reductions, oxidations, alkylations, acylations, hydrolyses, esterifications, and etherifications. The precursor groups can be changed to a different such group, or to the groups defined in formula I, at any time during the reaction sequence. For example, in cases where Y is —C(O)OR^{9b} and R^{9b} does not initially represent hydrogen (so providing an ester functional group), the skilled person will appreciate that at any stage during the synthesis (e.g. the final step), the relevant substituent may be hydrolysed to form a carboxylic acid functional group (in which case R^{9b} will be hydrogen). In this respect, the skilled person may also refer to “Comprehensive Organic Functional Group Transformations” by A. R. Katritzky, O. Meth-Cohn and C. W. Rees, Pergamon Press, 1995.

Compounds of the invention may be isolated from their reaction mixtures using conventional techniques.

It will be appreciated by those skilled in the art that, in the processes described above and hereinafter, the functional groups of intermediate compounds may need to be protected by protecting groups.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Chemistry”, edited by J W F McOmie, Plenum Press (1973), and “Protective Groups in Organic Synthesis”, 3^{rd} edition, T. W. Greene & P. G. M. Wutz, Wiley-Interscience (1999).

### Medical and Pharmaceutical Uses

Compounds of the invention are indicated as pharmaceuticals. According to a further aspect of the invention there is provided a compound of the invention, as hereinbefore defined but without the proviso, for use as a pharmaceutical.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised) may therefore be described as “prodrugs” of compounds of the invention.

By “prodrug of a compound of the invention”, we include compounds that form a compound of the invention, in an experimentally-detectable amount, within a predetermined time (e.g. about 1 hour), following oral or parenteral administration. All prodrugs of the compounds of the invention are included within the scope of the invention.

Furthermore, certain compounds of the invention (including, but not limited to, compounds of formula I in which Y represents —C(O)OR^{9b} and R^{9b} is other than hydrogen) may possess no or minimal pharmacological activity as such, but may be administered parenterally or orally, and thereafter be metabolised in the body to form compounds of the invention that possess pharmacological activity as such (including, but not limited to, corresponding compounds of formula I, in which R^{9b} represents hydrogen). Such compounds (which also includes compounds that may possess some pharmacological activity, but that activity is appreciably lower than that of the “active” compounds of the invention to which they are metabolised), may also be described as “prodrugs”.

Thus, the compounds of the invention are useful because they possess pharmacological activity, and/or are metabolised in the body following oral or parenteral administration to form compounds which possess pharmacological activity.

Compounds of the invention are particularly useful because they may inhibit the activity of a member of the MAPEG family.

Compounds of the invention are particularly useful because they may inhibit (for example selectively) the activity of prostaglandin E synthases (and particularly microsomal prostaglandin E synthase-1 (mPGES-1)), i.e. they prevent the action of mPGES-1 or a complex of which the mPGES-1 enzyme forms a part, and/or may elicit a mPGES-1 modulating effect, for example as may be demonstrated in the test described below. Compounds of the invention may thus be useful in the treatment of those conditions in which inhibition of a PGES, and particularly mPGES-1, is required.

Compounds of the invention may inhibit the activity of leukotriene C₄ (LTC₄), for example as may be shown in a test such as that described in Eur. J. Biochem., 208, 725-734 (1992), and may thus be useful in the treatment of those conditions in which inhibition of LTC₄ is required. Compounds of the invention may also inhibit the activity of 5-lipoxygenase-activating protein (FLAP), for example as may be shown in a test such as that described in Mol. Pharmacol., 41, 873-879 (1992).

Compounds of the invention are thus expected to be useful in the treatment of inflammation.

The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain, pain generally and/or fever.

Accordingly, compounds of the invention may be useful in the treatment of asthma, chronic obstructive pulmonary disease, pulmonary fibrosis, inflammatory bowel disease, irritable bowel syndrome, inflammatory pain, fever, migraine, headache, low back pain, fibromyalgia, myofascial disorders, viral infections (e.g. influenza, common cold, herpes zoster, hepatitis C and AIDS), bacterial infections, fungal infections, dysmenorrhea, burns, surgical or dental procedures, malignancies (e.g. breast cancer, colon cancer, and prostate cancer), hyperprostaglandin E syndrome, classic Bartter syndrome, atherosclerosis, gout, arthritis, osteoarthritis, juvenile arthritis, rheumatoid arthritis, rheumatic fever, ankylosing spondylitis, Hodgkin's disease, systemic lupus erythematosus, vasculitis, pancreatitis, nephritis, bursitis, conjunctivitis, iritis, scleritis, uveitis, wound healing, dermatitis, eczema, psoriasis, stroke, diabetes mellitus, neurodegenerative disorders such as Alzheimer's disease and multiple sclerosis, autoimmune diseases, allergic disorders, rhinitis, ulcers, coronary heart disease, sarcoidosis and any other disease with an inflammatory component.

Compounds of the invention may also have effects that are, not linked to inflammatory mechanisms, such as in the reduction of bone loss in a subject. Conditions that may be mentioned in this regard include osteoporosis, osteoarthritis, Paget's disease and/or periodontal diseases. Compounds the invention may thus also be useful in increasing bone mineral density, as well as the reduction in incidence and/or healing of fractures, in subjects.

Compounds of the invention are indicated both in the therapeutic and/or prophylactic treatment of the above-mentioned conditions.

According to a further aspect of the present invention, there is provided a method of treatment of a disease which is associated with, and/or which can be modulated by inhibition of, a member of the MAPEG family such as a PGES (e.g. mPGES-1), LTC₄ and/or FLAP and/or a method of treatment of a disease in which inhibition of the activity of a member of the MAPEG family such as PGES (and particularly mPGES-1), LTC₄ and/or FLAP is desired and/or required (e.g. inflammation), which method comprises administration of a therapeutically effective amount of a compound of the invention, as hereinbefore defined but without the proviso, to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of the invention will normally be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of the invention, as hereinbefore defined but without the proviso, in admixture with a pharmaceutically acceptable adjuvant, diluent or carrier.

Compounds of the invention may also be combined with other therapeutic agents that are useful in the treatment of inflammation (e.g. NSAIDs and coxibs).

According to a further aspect of the invention, there is provided a combination product comprising:
(A) a compound of the invention, as hereinbefore defined but without the proviso; and
(B) another therapeutic agent that is useful in the treatment of inflammation, wherein each of components (A) and (B) is formulated in admixture with a pharmaceutically-acceptable adjuvant, diluent or carrier.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:

(1) a pharmaceutical formulation including a compound of the invention, as hereinbefore defined but without the proviso, another therapeutic agent that is useful in the treatment of inflammation, and a pharmaceutically-acceptable adjuvant, diluent or carrier; and
(2) a kit of parts comprising components:
(a) a pharmaceutical formulation including a compound of the invention, as hereinbefore defined but without the proviso, in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
   which components (a) and (b) are each provided in a form that is suitable for administration in conjunction with the other.

Compounds of the invention may be administered at varying doses. Oral, pulmonary and topical dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 500 mg, and preferably between about 1 mg to about 100 mg, of the active ingredient. Intravenously, the most preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Compounds of the invention may have the advantage that they are effective, and preferably selective, inhibitors of a member of MAPEG family, e.g. inhibitors of prostaglandin E synthases (PGES) and particularly microsomal prostaglandin E synthase-1 (mPGES-1). The compounds of the invention may reduce the formation of the specific arachidonic acid metabolite PGE₂ without reducing the formation of other COX generated arachidonic acid metabolites, and thus may not give rise to the associated side-effects mentioned hereinbefore.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

### Biological Test

In the assay mPGES-1 catalyses the reaction where the substrate PGH₂ is converted to PGE₂. mPGES-1 is expressed in E. coli and the membrane fraction is dissolved in 20 mM NaPi-buffer pH 8.0 and stored at −80° C. In the assay mPGES-1 is dissolved in 0.1M KPi-buffer pH 7.35 with 2.5 mM glutathione. The stop solution consists of H₂O/MeCN (7/3), containing FeCl₂ (25 mM) and HCl (0.15 M). The assay is performed at room temperature in 96-well plates. Analysis of the amount of PGE₂ is performed with reversed phase HPLC (Waters 2795 equipped with a 3.9×150 mm C18 column). The mobile phase consists of H₂O/MeCN (7/3), containing TFA (0.056%), and absorbance is measured at 195 nm with a Waters 2487 UV-detector.

The following is added chronologically to each well:
1. 100 μL mPGES-1 in KPi-buffer with glutathione. Total protein concentration: 0.02 mg/mL.
2. 1 μL inhibitor in DMSO. Incubation of the plate at room temperature for 25 minutes.
3. 4 μL of a 0.25 mM PGH₂ solution. Incubation of the plate at room temperature for 60 seconds.
4. 100 μL stop solution.
   180 μL per sample is analyzed with HPLC.

### EXAMPLES

The invention is illustrated by way of the following examples, in which the following abbreviations may be employed:

cy cyclohexyl
dba dibenzylideneacetone
DIBAL diisobutylaluminium hydride
DMAP 4,4-dimethylaminopyridine
DMF dimethylformamide
DMSO dimethylsulfoxide
DPEphos bis-(2-diphenylphosphinophenyl)ether
EtOAc ethyl acetate

### HPLC High Pressure Liquid Chromatography

MeCN acetonitrile
MS mass spectrum
NMR nuclear magnetic resonance
rt room temperature
TFA trifluoroacetic acid
THF tetrahydrofuran
TMEDA N,N,N′,N′-tetramethylethylendiamine
xantphos 9,9-dimethyl-4,5-bis(diphenylphosphino)-xanthene

Starting materials and chemical reagents specified in the syntheses described below are commercially available from, e.g. Sigma-Aldrich Fine Chemicals.

### Example 1

### 5-(4-tert-Butylphenyl)-3-formyl-1-(4-isopropoxyphenyl)indole-2-carboxylic acid

### (a) 5-Bromo-3-formylindole-2-carboxylic acid ethyl ester

Oxalyl chloride (3.43 mL, 39.9 mmol) was added to a stirred solution of DMF (30 mL) in CH₂Cl₂ (80 mL) at 0° C. After 20 min at 0° C. for, a solution of 5-bromo-indole-2-carboxylic acid ethyl ester (10 g, 37.3 mmol) in DMF (80 mL) was added. After 24 h at rt the mixture was poured into NaHCO₃ (aq, sat) and extracted with CH₂Cl₂. The combined extracts were washed with water and brine, dried (Na₂SO₄), concentrated and the purified by crystallisation from EtOH to give the sub-title compound (8.9 g, 81%).

### (b) 5-Bromo-3-formyl-1-(4-isopropoxyphenyl)indole-2-carboxylic acid ethyl ester

Anhydrous CH₂Cl₂ (100 mL), Et₃N (3.8 mL, 27.02 mmol), pyridine (2.2 mL, 27.02 mmol) and 3 Å molecular sieves (ca. 5 g) were added to 5-bromo-3-formylindole-2-carboxylic acid ethyl ester (4 g, 13.51 mmol; see step (a) above), Cu(OAc)₂ (4.91 g, 27.02 mmol) and 4-isopropoxyphenylboronic acid (4.86 g, 27.02 mmol). The mixture was stirred vigorously at rt for 30 h and filtered through Celite®. The solids were washed with EtOAc, and the combined filtrates concentrated and purified by chromatography to afford the sub-title compound (4.1 g, 71%).

### (c) 5-(4-tert-Butylphenyl)-3-formyl-1-(4-isopropoxyphenyl)indole-2-carboxylic acid ethyl ester

A mixture of 5-bromo-3-formyl-1-(4-isopropoxyphenyl)indole-2-carboxylic acid ethyl ester (4.07 g, 9.46 mmol; see step (b) above), 4-tert-butylphenylboronic acid (2.53 g, 14.19 mmol), K₃PO₄ (7.03 g, 33.10 mmol), Pd(OAc)₂ (106 mg, 0.47 mmol), tri-o-tolylphosphine (288 mg, 0.95 mmol), EtOH (10 ml) and toluene (40 mL) was stirred under argon for 20 min at rt, and then heated at 100° C. for 50 min. The mixture was cooled to rt, poured into NaHCO₃ (aq, sat) and extracted with EtOAc. The combined extracts were washed with water and brine, dried (Na₂SO₄), concentrated and purified by chromatography to give the sub-title compound (4.16 g, 91%).

### (d) 5-(4-tert-Butylphenyl)-3-formyl-1-(4-isopropoxyphenyl)indole-2-carboxylic acid

5-(4-tert-Butylphenyl)-3-formyl-1-(4-isopropoxyphenyl)indole-2-carboxylic acid ethyl ester (see step (c)) was hydrolysed in accordance with Example 2, step (b).

### Example 2

### 5-(4-tert-Butylphenyl)-1-(4-isopropoxyphenyl)-3-morpholin-4-ylmethylindole-2-carboxylic acid

### (a) 5-(4-tert-Butylphenyl)-1-(4-isopropoxyphenyl)-3-morpholin-4-yl-methylindole-2-carboxylic acid ethyl ester

Morpholine (146 4, 1.66 mmol) was added to a suspension of 5-(4-tert-butylphenyl)-3-formyl-1-(4-isopropoxyphenyl)indole-2-carboxylic acid ethyl ester (400 mg, 0.83 mmol; see Example 1, step (c)) in MeOH (20 mL) and the pH was adjusted to 6 by the dropwise addition of glacial acetic acid. After 1 h at rt, NaCNBH₃ (75 mg, 1.18 mmol) was added and the mixture was stirred at rt for 24 h, poured into water and extracted with EtOAc. The combined extracts were washed with water and brine, dried (Na₂SO₄), concentrated and purified by chromatography to give the sub-title compound (400 mg, 87%).

### (b) 5-(4-tert-Butylphenyl)-1-(4-isopropoxyphenyl)-3-morpholin-4-ylmeth-yl-indole-2-carboxylic acid

A mixture of 5-(4-tert-butylphenyl)-1-(4-isopropoxyphenyl)-3-morpholin-4-yl-methylindole-2-carboxylic acid ethyl ester (198 mg, 0.36 mmol, see step (a)), NaOH (aq, 1 M, 2 mL) and dioxane (3 mL) was heated at 120° C. for 30 min. The mixture was acidified with HCl (1 M) to pH 5 and extracted with EtOAc. The combined extracts were washed with water and brine, dried (Na₂SO), concentrated and purified by chromatography. Crystallisation from MeOH afforded the title compound (110 mg, 59%).

¹H NMR (DMSO-d₆, 200 MHz): δ 8.09-8.05 (1H, m), 7.66-7.58 (2H, m), 7.55-7.44 (3H, m), 7.27-7.18 (2H, m), 7.09-6.97 (3H, m), 4.68 (1H, septet, J=6.0 Hz), 4.37 (2H, s), 3.79-3.66 (4H, m), 3.02-2.89 (4H, m), 1.33 (6H, d, J=6.0 Hz), 1.32 (9H, s).

### Example 3

### 5-(4-tert-Butylphenyl)-1-(4-isopropoxyphenyl)-3-(4-methylpiperazin-1-ylmethyl)-indole-2-carboxylic acid

The title compound was prepared in accordance with Example 2 from 5-(4-tert-butylphenyl)-3-formyl-1-(4-isopropoxyphenyl)indole-2-carboxylic acid ethyl ester and N-methylpiperazine, followed by hydrolysis (see Example 2 (b)).

¹H NMR (DMSO-d₆, 200 MHz): δ17.0-16.0 (1H, br s), 8.07-8.02 (1H, m), 7.65-7.58 (2H, m), 7.53-7.44 (3H, m), 7.24-7.16 (2H, m), 7.08-6.95 (3H, m), 4.67 (1H, septet, J=6.0 Hz), 4.41 (2H, s), 3.18-2.87 (4H, m), 2.70-2.30 (4H, m, overlapped with DMSO signal), 2.23 (3H, s), 1.33 (6H, d, J=6.0 Hz) 1.32 (9H, s).

### Example 4

### 5-(4-tert-Butylphenyl)-1-(4-isopropoxyphenyl)-3-{[(pyridin-2-ylmethyl)-amino]methyl}indole-2-carboxylic acid

The title compound was prepared in accordance with Example 2 from 5-(4-tert-butylphenyl)-3-formyl-1-(4-isopropoxyphenyl)indole-2-carboxylic acid ethyl ester and 2-(aminomethyl)pyridine, followed by hydrolysis (see Example 2 (b)).

¹H NMR (DMSO-d₆, 200 MHz): δ 12.1-11.2 (1H, br s), 8.66-8.60 (1H, m), 7.99-7.95 (1H, m), 7.85 (1H, ddd, J=7.8, 7.8. 1.7 Hz), 7.65-7.56 (2H, m), 7.52-7.36 (5H, m), 7.22-7.13 (2H, m), 7.05 (1H, d, J=8.7 Hz), 7.02-6.95 (2H, m), 4.66 (1H, septet, J=6.0 Hz), 4.50 (2H, s), 4.28 (2H, s), 1.33 (6H, d, J=6.0 Hz), 1.32 (9H, s).

### Example 5

### [5-(4-tert-Butylphenyl)-2-carboxy-1-(4-isopropoxyphenyl)indol-3-ylmethyl]-(2-hydroxyethyl)ammonium chloride

### (a) 5-(4-tert-Butylphenyl)-3-[(2-hydroxyethylamino)methyl]-1-(4-isopropoxyphenyl)-indole-2-carboxylic acid

The sub-title compound was prepared in accordance with Example 2 from 5-(4-tert-butylphenyl)-3-formyl-1-(4-isopropoxyphenyl)indole-2-carboxy-lic acid ethyl ester and 2-aminoethanol, followed by hydrolysis (see Example 2 (b)).

### (b) [5-(4-tert-Butylphenyl)-2-carboxy-1-(4-isopropoxyphenyl)indol-3-ylmethyl]-(2-hydroxyethyl)ammonium chloride

5-(4-tert-Butylphenyl)-3-[(2-hydroxyethylamino)methyl]-1-(4-isopropoxy-phenyl)indole-2-carboxylic acid (189 mg, 0.38 mmol; see step (a) above) was suspended in dioxane (4 mL) and an excess HCl (4 M in dioxane) was added. After 10 min the mixture was concentrated and the residue treated with ether and filtered to give the title compound.

¹H NMR (DMSO-d₆, 200 MHz): δ 13.2-13.8 (1H, br s), 9.1 (2H, br s), 8.32-8.28 (1H, m), 7.73-7.60 (3H, m), 7.53-7.46 (2H, m), 7.31-7.23 (2H, m), 7.12-7.03 (3H, m), 5.39-5.19 (1H, m), 4.73 (2H, s), 4.70 (1H, septet, J=6.0 Hz), 3.78-3.67 (2H, m), 3.19-3.05 (2H, m), 1.34 (6H, d, J=6.0 Hz), 1.33 (9H, s).

### Example 6

### [5-(4-tert-Butylphenyl)-2-carboxy-1-(4-isopropoxyphenyl)indol-3-ylmethyl]-(2-hydroxy-1-hydroxymethylethyl)ammonium chloride

The title compound was prepared in accordance with Example 2 from 5-(4-tert-butylphenyl)-3-formyl-1-(4-isopropoxyphenyl)indole-2-carboxylic acid ethyl ester and 2-aminopropane-1,3-diol followed by hydrolysis (see Example 2 (b)) and followed by salt formation (see Example 5, step (b)).

¹H NMR (DMSO-d₆, 200 MHz): δ 14.1-13.3 (1H, br s), 9.00-8.76 (2H, m), 8.32-8.24 (1H, m), 7.72-7.60 (3H, m), 7.54-7.46 (2H, m), 7.32-7.23 (2H, m), 7.13-7.03 (3H, m), 5.5-5.3 (2H, m), 4.87-4.74 (2H, m), 4.71 (1H, septet, J=6.0 Hz), 3.86-3.64 (4H, m), 3.32-3.16 (1H, m, overlapped with H₂O), 1.34 (6H, d, J=6.0 Hz), 1.33 (9H, s).

### Example 7

### (2-{[5-(4-tert-Butylphenyl)-2-carboxy-1-(4-isopropoxyphenyl)indol-3-ylmethyl]-amino}ethyl)dimethylammonium dichloride

The title compound was prepared in accordance with Example 2 from 5-(4-tert-butylphenyl)-3-formyl-1-(4-isopropoxyphenyl)indole-2-carboxylic acid ethyl ester and N,N-dimethylethylenediamine, followed by hydrolysis (see Example 2 (b)) followed by salt formation (see Example 5, step (b)).

¹H NMR (DMSO-d₆, 200 MHz): δ 14.0-13.0 (1H, br s), 11.5-10.3 (1H, br s), 10.1-9.0 (2H, br s), 8.37-8.31 (1H, m), 7.76-7.66 (2H, m), 7.63 (1H, dd, J=8.9, 1.4 Hz), 7.52-7.43 (2H, m), 7.31-7.22 (2H, m), 7.12-7.02 (3H, m), 4.75 (2H, s), 4.69 (1H, septet, J=6.0 Hz), 3.61-3.45 (4H, m), 2.83 (6H, s), 1.32 (6H, d, J=6.0 Hz), 1.31 (9H, s).

### Example 8

### 5-(4-tert-Butylphenyl)-3-dimethylaminomethyl-1-(4-isopropoxyphenyl)indole-2-carboxylic acid

### (a) 5-(4-tert-Butylphenyl)-3-dimethylaminomethyl-1-(4-isopropoxyphen-yl)-indole-2-carboxylic acid ethyl ester

A mixture of 5-(4-tert-butylphenyl)-3-formyl-1-(4-isopropoxyphenyl)indole-2-carboxylic acid ethyl ester (500 mg, 1.03 mmol; see Example 1, step (c)), dimethyl ammonium chloride (165 mg, 2.03 mmol), sodium acetate (134 mg, 1.63 mmol) and MeOH (20 mL) was stirred for 1 h at rt. NaCNBH₃ (93 mg, 1.48 mmol) was added and the mixture was stirred at rt for 24 h, poured into water and extracted with EtOAc. The combined extracts were washed with water and brine, dried (Na₂SO₄), concentrated and purified by chromatography to give the sub-title compound (410 mg, 78%).

### (b) 5-(4-tert-Butylphenyl)-3-dimethylaminomethyl-1-(4-isopropoxyphenyl)indole-2-carboxylic acid

The title compound was prepared in accordance with Example 2, step (b) from 5-(4-tert-butylphenyl)-3-dimethylaminomethyl-1-(4-isopropoxy-phenyl)indole-2-carboxylic acid ethyl ester.

¹H NMR (DMSO-d₆, 200 MHz): 815.5-14.5 (1H, br s), 8.04-8.00 (1H, m), 7.66-7.58 (2H, m), 7.52-7.43 (3H, m), 7.23-7.15 (2H, m), 7.05 (1H, d, J=8.8 Hz), 7.02-6.95 (2H, m), 4.66 (1H, septet, J=6.0 Hz), 4.44 (2H, s), 2.72 (6H, s), 1.33 (6H, d, J=6.0 Hz), 1.32 (9H, s).

### Example 9

### 3-[(1,3-dihydroxypropan-2-ylamino)methyl]-1-(4-isopropoxyphenyl)-5-(5-trifluoromethylpyridin-2-yl)indole-2-carboxylic acid dihydrochloride

### (a) 3-Formyl-1-(4-isopropoxyphenyl)-5-(4,4,5,5-tetramethyl-[1,3,2]-dioxaborolan-2-yl)indole-2-carboxylic acid ethyl ester

Pd₂(dba)₃ (0.31 g, 0.034 mmol) and tricyclohexylphosphine (57 mg, 0.20 mmol) in dioxane (3.4 mL) were added under argon to a stirred mixture of 5-bromo-3-formyl-1-(4-isopropoxyphenyl)indole-2-carboxylic acid ethyl ester (581 mg, 1.35 mmol, see Example 1, step (b)), KOAc (198 mg, 2.02 mmol), bis(pinacolato)diboron (375 mg, 1.46 mmol) and dioxane (10 mL) at 80° C. The mixture was stirred at 80° C. for 24 h, allowed to cool and filtered through Celite®. The solids were washed with EtOAc and the combined filtrates were concentrated and purified by chromatography to yield the sub-title compound (600 g, 93%).

### (b) 3-Formyl-1-(4-isopropoxyphenyl)-5-(5-trifluoromethylpyridin-2-yl)indole-2-carboxylic acid ethyl ester

A stirred mixture of 3-formyl-1-(4-isopropoxyphenyl)-5-(4,4,5,5-tetramethyl-[1,3,2]-dioxaborolan-2-yl)indole-2-carboxylic acid ethyl ester (600 mg, 1.26 mmol; see step (a)), 2-bromo-5-(trifluoromethyl)pyridine (426 mg, 1.89 mmol), Na₂CO₃ (aq, 2 M, 1.89 mL, 3.78 mmol), Pd(PPh₃)₄ (70 mg, 0.06 mmol), EtOH (5 mL) and toluene (20 mL) was heated at 80° C. for 24 h. The mixture was allowed to cool, poured into water and extracted with EtOAc. The combined extracts were washed with water and brine, dried (Na₂SO₄), concentrated and purified by chromatography to give the sub-title compound (500 mg, 80%).

### (c) 3-[(2-Hydroxy-1-hydroxymethylethylamino)methyl]-1-(4-isopropoxyphenyl)-5-(5-trifluoromethylpyridin-2-yl)indole-2-carboxylic acid ethyl ester

The sub-title compound was prepared in accordance with Example 2 step (a) from 3-formyl-1-(4-isopropoxyphenyl)-5-(5-trifluoromethylpyridin-2-yl)indole-2-carboxylic acid ethyl ester and 2-aminopropane-1,3-diol.

### (d) 3-[(2-Hydroxy-1-hydroxymethylethylamino)methyl]-1-(4-isopropoxyphenyl)-5-(5-trifluoromethylpyridin-2-yl)indole-2-carboxylic acid

The sub-title compound was prepared in accordance with Example 2, step (b) from 3-[(2-hydroxy-1-hydroxymethylethylamino)methyl]-1-(4-isopropoxyphenyl)-5-(5-trifluoromethylpyridin-2-yl)indole-2-carboxylic acid ethyl ester.

### (e) 3-[(2-Hydroxy-1-hydroxymethylethylamino)methyl]-1-(4-isopropoxyphenyl)-5-(5-trifluoromethylpyridin-2-yl)indole-2-carboxylic acid dihydrochloride

The title compound was prepared in accordance with Example 5 step (b) from 3-[(2-hydroxy-1-hydroxymethylethylamino)methyl]-1-(4-isopropoxyphenyl)-5-(5-trifluoromethylpyridin-2-yl)indole-2-carboxylic acid.

¹H NMR (DMSO-d₆, 200 MHz): δ 9.05 (1H, s), 8.9-8.7 (2H, br s), 8.91-8.84 (1H, m), 8.38-8.31 (2H, m), 8.26-8.18 (1H, m), 7.35-7.25 (2H, m), 7.18 (1H, d, J=8.9 Hz), 7.13-7.05 (2H, m), 4.91-4.79 (2H, m), 4.71 (1H, septet, J=6.0 Hz), 3.86-3.08 (7H, m, overlapped with H₂O), 1.34 (6H, d, J=6.0 Hz).

### Example 10

### 1-(4-Isopropoxyphenyl)-3-(4-methylpiperazin-1-ylmethyl)-5-(5-trifluoromethyl-pyridin-2-yl)indole-2-carboxylic acid trihydrochloride

The title compound was prepared in accordance with Example 9 from 3-formyl-1-(4-isopropoxyphenyl)-5-(5-trifluoromethylpyridin-2-yl)indole-2-carboxylic acid ethyl ester (see Example 9, step (b)) and N-methylpiperazine.

¹H NMR (DMSO-d₆, 200 MHz): δ 12.5-11.0 (1H, br s), 9.06-9.00 (1H, m), 8.95 (1H, s), 8.46 (1H, d, J=8.5 Hz), 8.32 (1H, dd, J=8.5, 2.0 Hz), 8.23 (1H, dd, J=8.8, 1.4 Hz), 7.39-7.30 (2H, m), 7.18 (1H, d, J=8.8 Hz), 7.12-7.04 (2H, m), 4.91 (2H, s), 4.71 (1H, septet, J=6.0 Hz), 3.82-3.37 (8H, m), 2.81 (3H, s), 1.34 (6H, d, J=6.0 Hz).

### Example 11

### 3-(2-Cyanoethyl)-1-(4-cyclopentyloxyphenyl)-5-(4-trifluoromethylphenyl)indole-2-carboxylic acid

### (a) 5-(4-Trifluoromethylphenyl)indole-2-carboxylic acid ethyl ester

A mixture of 5-bromoindole-2-carboxylic acid ethyl ester (4.22 g, 16 mmol), 4-trifluoromethylphenylboronic acid (4.50 g, 24 mmol), K₃PO₄ (11.7 g, 55 mmol), Pd(OAc)₂ (176 mg, 0.78 mmol), tri-o-tolylphosphine (478 mg, 1.6 mmol), EtOH (20 ml) and toluene (90 mL) was stirred under argon for 20 min at rt followed by heating at 100° C. for 2 h. The mixture was cooled to rt, poured into NaHCO₃ (aq, sat) and extracted with EtOAc. The combined extracts were washed with water and brine, dried (Na₂SO₄), concentrated and purified by chromatography to yield the sub-title compound (3.91 g, 75%).

### (b) 3-Iodo-5-(4-trifluoromethylphenyl)indole-2-carboxylic acid ethyl ester

A solution of NaI (2.04 g, 14 mmol) in acetone (10 mL) was added dropwise to a stirred solution of N-chlorosuccinimide (1.83 g, 14 mmol) in acetone (10 mL) protected from light. After 15 min, a solution of 5-(4-trifluoromethylphenyl)-indole-2-carboxylic acid ethyl ester (3.80 g, 11 mmol; see step (a) above), in acetone (60 mL) was added dropwise, followed by stirring for 2 h at rt. The mixture was poured into Na₂S₂O₃ (aq, 10%, 250 mL) and extracted with EtOAc (2×200 mL). The combined extracts were washed with NaHCO₃ (aq, sat), water and brine, dried (Na₂SO₄) and concentrated. The residue was washed with petroleum ether to give sub-title compound (4.88 g, 93%).

### (c) 1-(4-Cyclopentyloxyphenyl)-3-iodo-5-(4-trifluoromethylphenyl)indole-2-carboxylic acid ethyl ester

Anhydrous CH₂Cl₂ (110 mL), Et₃N (2.45 mL, 17.4 mmol) and pyridine (1.42 mL, 17.4 mmol) were added to 3-iodo-5-(4-trifluoromethyl-phenyl)indole-2-carboxylic acid ethyl ester (4.00 g, 8.72 mmol; see step (b) above), Cu(OAc)₂ (3.16 g, 17.4 mmol), 3 Å molecular sieves (ca. 8 g) and 4-cyclopentyloxyphenylboronic acid (3.59 g, 17.48 mmol). The mixture was stirred vigorously at rt for 120 h and filtered through Celite®. The solids were washed with EtOAc and the combined filtrates concentrated and purified by chromatography to afford the sub-title compound (3.83 g, 71%).

### (d) 3-(2-Cyanovinyl)-1-(4-cyclopentyloxyphenyl)-5-(4-trifluoromethylphenyl)-indole-2-carboxylic acid ethyl ester

A mixture of 1-(4-cyclopentyloxyphenyl)-3-iodo-5-(4-trifluoromethylphenyl)-indole-2-carboxylic acid ethyl ester (217 mg, 0.35 mmol; see step (c)), acrylonitrile (30 μL, 0.44 mmol), Pd(OAc)₂ (3.9 mg, 0.018 mmol), diisopropylethyl-amine (60 μL, 0.35 mmol) and DMF (1.0 mL) was stirred for 20 min at 120° C. and cooled to rt. The mixture was diluted with EtOAc and washed with NaHCO₃ (aq, 5%), HCl (aq, 0.5 M), water and brine, dried (Na₂SO₄), concentrated and purified by chromatography to give the sub-title compound (124 mg, 65%).

### (e) 3-(2-Cyanoethyl)-1-(4-cyclopentyloxyphenyl)-5-(4-trifluoromethylphenyl)-indole-2-carboxylic acid ethyl ester

3-(2-Cyanovinyl)-1-(4-cyclopentyloxyphenyl)-5-(4-trifluoromethylphenyl)indole-2-carboxylic acid ethyl ester ((118 mg, 0.22 mmol; see step (d)) dissolved in a mixture of MeOH and THF was hydrogenated (rt, 5 bar) over 10% Pd/C. The mixture was filtered through Celite®, concentrated and purified by chromatography to give the sub-title compound (100 mg, 84%).

### (f) 3-(2-Cyanoethyl)-1-(4-cyclopentyloxyphenyl)-5-(4-trifluoromethylphenyl)-indole-2-carboxylic acid

A mixture of 3-(2-cyanoethyl)-1-(4-cyclopentyloxyphenyl)-5-(4-trifluoromethylphenyl)indole-2-carboxylic acid ethyl ester (94 mg, 0.17 mmol; see step (e) above), NaOH (69 mg, 1.7 mmol, in 1.0 mL water) and MeCN (2 mL) was heated for 20 min at 120° C., cooled, acidified with HCl (1 M) to pH 2 and extracted with EtOAc. The combined extracts were washed with water and brine, dried (Na₂SO₄), concentrated and purified by chromatography. The crude product was crystallised and then recrystallised from EtOH to give the title compound (82 mg, 93%).

200 MHz ¹H-NMR (DMSO-d₆, ppm) δ 13.1-13.0 (1H, br s), 8.27 (1H, s), 8.01-7.91 (2H, m), 7.85-7.75 (2H, m), 7.65 (1H, dd, J=8.7 1.3 Hz), 7.29-7.18 (2H, m), 7.08 (1H, d, J=8.7 Hz), 7.06-6.96 (2H, m), 4.93-4.81 (1H, m), 3.49 (2H, t, J=7.2 Hz), 2.88 (2H, t, J=7.2 Hz), 2.05-1.50 (8H, m).

### Example 12

### 1-(4-Cyclopentyloxyphenyl)-3-(2-pyridin-4-yl-ethyl)-5-(4-trifluoromethylphenyl)-indole-2-carboxylic acid

### (a) 1-(4-Cyclopentyloxyphenyl)-34(E)-2-pyridin-4-yl-vinyl)-5-(4-trifluoro-methylphenyl)indole-2-carboxylic acid ethyl ester

A mixture of 1-(4-cyclopentyloxyphenyl)-3-iodo-5-(4-trifluoromethylphenyl)-indole-2-carboxylic acid ethyl ester (250 mg, 0.40 mmol; see Example 11, step (c)), 4-vinylpyridine (169 mg, 1.6 mmol), Pd(OAc)₂ (2.3 mg, 0.01 mmol), tri-o-tolylphosphine (6.7 mg, 0.022 mmol), Cs₂CO₃ (157 mg, 0.48 mmol), tetrabutylammonium bromide (130 mg, 0.40 mmol) and DMF (2.5 mL) was stirred for 8 min at 150° C. and cooled to rt. The mixture was diluted with EtOAc and washed with NaHCO₃ (aq, sat), HCl (aq, 0.1 M), water and brine, dried (Na₂SO₄), concentrated and purified by chromatography to yield the sub-title compound (144 mg, 60%).

### (b) 1-(4-Cyclopentyloxyphenyl)-3-(2-pyridin-4-ylethyl)-5-(4-trifluoromethyl-phenyl)indole-2-carboxylic acid ethyl ester

The sub-title compound (50 mg, 55%) was prepared in accordance with Example 11, step (e) from 1-(4-cyclopentyloxyphenyl)-3-(E)-2-pyridin-4-ylvinyl)-5-(4-trifluoromethylphenyl)indole-2-carboxylic acid ethyl ester (90 mg, 0.15 mmol; see step (a) above).

### (c) 1-(4-Cyclopentyloxyphenyl)-3-(2-pyridin-4-ylethyl)-5-(4-trifluoromethylphenyl)indole-2-carboxylic acid

The title compound was prepared in accordance with Example 11 step (f) from 1-(4-cyclopentyloxyphenyl)-3-(2-pyridin-4-ylethyl)-5-(4-trifluoro-methylphenyl)-indole-2-carboxylic acid ethyl ester (46 mg, 0.077 mmol; see step (b) above). The crude product was purified by chromatography and repeated recrystallisation from EtOH to yield the title compound (44 mg, 100% yield).

200 MHz ¹H-NMR (DMSO-d₆, ppm) δ 13.0-12.8 (1H, br s), 8.45 (2H, d, J=4.4 Hz), 8.08 (1H, s), 7.96-7.85 (2H, m), 7.85-7.74 (2H, m), 7.61 (1H, d, J=8.8 Hz), 7.31 (2H, d, J=4.4 Hz), 7.28-7.17 (2H, m), 7.07 (1H, d, J=8.8 Hz), 7.05-6.96 (2H, m), 4.93-4.79 (1H, m), 3.55-3.36 (2H, m), 3.06-2.89 (2H, m), 2.06-1.50 (8H, m).

### Example 13

### 1-(4-Cyclopentyloxyphenyl)-3-[(E)-2-(4-methylthiazol-5-yl)vinyl]-5-(4-trifluoromethylphenyl)-indole-2-carboxylic acid

The title compound was prepared in accordance with Example 12 from 1-(4-cyclopentyloxyphenyl)-3-iodo-5-(4-trifluoromethylphenyl)indole-2-carboxylic acid ethyl ester and 4-methyl-5-vinylthiazole, followed by hydrolysis (see Example 11, step (f)).

200 MHz ¹H-NMR (DMSO-d₆, ppm) δ 13.3 (1H, br s), 8.89 (1H, s), 8.37 (1H, s), 8.02-7.92 (2H, m), 7.85-7.76 (2H, m), 7.68 (1H, d, J=8.8 Hz), 7.67 (1H, d, J=16.5 Hz), 7.53 (1H, d, J=16.5 Hz), 7.33-7.22 (2H, m), 7.14 (1H, d, J=8.8 Hz), 7.08-6.97 (2H, m), 4.93-4.81 (1H, m), 2.51 (3H, s), 2.06-1.50 (8H, m).

### Example 14

### 3-[2-Carboxy-1-(4-cyclopentyloxyphenyl)-5-(4-trifluoromethylphenyl)indol-3-yl]-propyl ammonium chloride

### (a) 3-(3-Aminopropyl)-1-(4-cyclopentyloxyphenyl)-5-(4-trifluoromethylphenyl)-indole-2-carboxylic acid ethyl ester

BH_{3*}THF (1 M in THF) was added to a mixture of 3-(2-cyanoethyl)-1-(4-cyclopentyloxyphenyl)-5-(4-trifluoromethylphenypindole-2-carboxylic acid ethyl ester (356 mg, 0.65 mmol; see Example 11, step (e)) and THF (4 mL) at 0° C. (ice bath) during 10 min. After 2 h at rt, the mixture was cooled to 0° C. and the pH was adjusted to 1 by addition of HCl (aq, 1 M). After 20 min the pH was adjusted to 10 with NaOH (aq). The mixture was diluted with water (10 mL) and extracted with Et₂O (3×20 mL). The combined extracts were washed with brine, dried (Na₂SO₄), concentrated and purified by chromatography to give the sub-title compound (135 mg, 38%).

### (b) 3-[2-Carboxy-1-(4-cyclopentyloxyphenyl)-5-(4-trifluoromethylphenyl)indol-3-yl]propyl ammonium chloride

A mixture of 3-(3-aminopropyl)-1-(4-cyclopentyloxyphenyl)-5-(4-trifluoromethylphenyl)indole-2-carboxylic acid ethyl ester (135 mg, 0.245 mmol, see step (a)), NaOH (98 mg, 2.45 mmol), EtOH (2 mL) and water (3 mL) was heated at reflux for 2 h. The mixture was filtered, acidified with HCl (aq) to pH 5 and extracted with EtOAc. The combined extracts were washed with brine, dried (Na₂SO₄), concentrated and purified by chromatography. The crude product was dissolved in CH₂Cl₂ (10 mL) and HCl (0.4 M in CH₂Cl₂, 0.85 mL) was added. The mixture was concentrated and crystallised from CH₂Cl₂ affording the title compound (44 mg, 32%).

200 MHz ¹H-NMR (DMSO-d₆, ppm) δ 13.2-12.8 (1H, br.s) 8.18-8.13 (1H, m) 8.05-7.74 (7H, m) 7.62 (1H, dd, J=8.5, 1.5 Hz) 7.28-7.16 (2H, m) 7.10 (1H, d, J=8.5 Hz) 7.05-6.94 (2H, m) 4.92-4.79 (1H, m) 3.26-3.11 (2H, m) 2.93-2.73 (2H, m) 2.08-1.47 (10H, m).

### Example 15

### 1-(4-Isopropoxyphenyl)-3-(2-pyridin-4-yl-ethyl)-5-(5-trifluoromethylpyridin-2-yl)indole-2-carboxylic acid

### (a) 5-(4,4,5,5-Tetramethyl-[1,3,2]dioxaborolan-2-yl)indole-2-carboxylic acid ethyl ester

Pd₂(dba)₃ (275 mg, 0.30 mmol) and tricyclohexylphosphine (504 mg, 1.80 mmol) in dioxane (30 mL) were added under argon to a stirred mixture of 5-bromoindole-2-carboxylic acid ethyl ester (6.0 g, 22.4 mmol), KOAc (3.3 g, 33.6 mmol), bis(pinacolato)diboron (6.3 g, 24.6 mmol) and dioxane (20 mL) at 80° C. The mixture was stirred at 80° C. for 3 h, cooled to rt and filtered through Celite®. The solids were washed with EtOAc and the combined filtrates were concentrated and purified by chromatography to yield the sub-title compound (6.8 g, 97%).

### (b) 5-(5-Trifluoromethylpyridin-2-yl)indole-2-carboxylic acid ethyl ester

A stirred mixture of 5-(4,4,5,5-tetramethyl-[1,3,2]dioxaborolan-2-yl)indole-2-carboxylic acid ethyl ester (3.00 g, 9.52 mmol; see step (a) above), 2-bromo-5-trifluoromethylpyridine (3.23 g, 14.28 mmol), Na₂CO₃ (aq, 2 M, 14.3 mL, 28.6 mmol), Pd(PPh₃)₄ (540 mg, 0.50 mmol), EtOH (10 mL) and toluene (40 mL) was heated at 80° C. for 24 h. The mixture was cooled to rt, poured into water and extracted with EtOAc. The combined extracts were washed with water, brine, dried (Na₂SO₄), concentrated and purified by chromatography yielding the sub-title compound (3.0 g, 94%).

### (c) 3-Indo-5-(5-trifluoromethylpyridin-2-yl)indole-2-carboxylic acid ethyl ester

The sub-title compound was prepared in accordance with the procedure described in Example 11 step (b) using 5-(5-trifluoromethylpyridin-2-yl)indole-2-carboxylic acid ethyl ester (see step (b) above).

### (d) 3-Iodo-1-(4-isopropoxyphenyl)-5-(5-trifluoromethylpyridin-2-yl)indole-2-carboxylic acid ethyl ester

The sub-title compound was prepared in accordance with the procedure described in Example 11 step (c) using 3-iodo-5-(5-trifluoromethylpyridin-2-yl)indole-2-carboxylic acid ethyl ester (see step (c) above) and 4-isopropoxyphenylboronic acid.

### (e) 1-(4-Isopropoxyphenyl)-3-((E)-2-pyridin-4-ylvinyl)-5-(5-trifluoromethylpyridin-2-yl)indole-2-carboxylic acid ethyl ester

The sub-title compound was prepared in accordance with the procedure described in Example 12 step (a) using 3-iodo-1-(4-isopropoxyphenyl)-5-(5-trifluoromethylpyridin-2-yl)indole-2-carboxylic acid ethyl ester (see step (d) above) and 4-vinylpyridine.

### (f) 1-(4-Isopropoxyphenyl)-3-(2-pyridin-4-ylethyl)-5-(5-trifluoromethylpyridin-2-yl)indole-2-carboxylic acid ethyl ester

The sub-title compound was prepared in accordance with Example 11, step (e) from 1-(4-isopropoxyphenyl)-3-((E)-2-pyridin-4-ylvinyl)-5-(5-trifluoromethylpyridin-2-yl)indole-2-carboxylic acid ethyl ester (168 mg, 0.29 mmol; see step (e) above) to give (141 mg, 84%).

### (g) 1-(4-Isopropoxyphenyl)-3-(2-pyridin-4-ylethyl)-5-(5-trifluoromethylpyridin-2-yl)indole-2-carboxylic acid

A mixture of 1-(4-isopropoxyphenyl)-3-(2-pyridin-4-yl-ethyl)-5-(5-trifluoromethylpyridin-2-yl)indole-2-carboxylic acid ethyl ester (133 mg, 0.23 mmol; see step (f) above), NaOH (46 mg, 1.2 mmol, in 1.5 mL water) and EtOH (2.5 mL) was heated at reflux for 2.5 h, cooled to rt, acidified with HCl (aq, 1M) to pH 5.6 and extracted with EtOAc. The combined extracts were washed with brine, dried (Na₂SO₄), concentrated and purified by chromatography affording the title compound (105 mg, 77%).

200 MHz ¹H-NMR (DMSO-d₆, ppm) δ 13.0-12.9 (1H, br s) 8.99 (1H, s) 8.56-8.50 (1H, m) 8.50-8.40 (2H, m) 8.30-8.20 (2H, m) 8.11 (1H, dd, J=8.8, 1.4 Hz) 7.35-7.18 (4H, m) 7.10 (1H, d, J=8.8 Hz) 7.08-6.97 (2H, m) 4.67 (1H, septet, J=6.0 Hz) 3.54-3.38 (2H, m) 3.07-2.91 (2H, m) 1.31 (6H, d, J=6.0 Hz).

### Example 16

### 1-(4-Isopropoxyphenyl)-3-((E)-2-pyridin-4-ylvinyl)-5-(5-trifluoromethylpyridin-2-yl)indole-2-carboxylic acid

The title compound was prepared in accordance with Example 15, step (g) from 1-(4-isopropoxyphenyl)-3-((E)-2-pyridin-4-yl-vinyl)-5-(5-trifluoromethylpyridin-2-yl)indole-2-carboxylic acid ethyl ester (Example 15, step (e)).

200 MHz ¹H-NMR for E isomer (DMSO-d₆, ppm) δ 9.04 (1H, s) 8.86 (1H, s) 8.58-8.49 (1H, m) 8.84 (1H, d, J=16.8 Hz) 8.31 (1H, d, J=8.6 Hz) 8.23 (1H, dd, J=8.6, 2.0 Hz) 8.04 (1H, d, J=8.8 Hz) 7.75 (1H, ddd, J=7.6, 7.6, 1.3 Hz) 7.56 (1H, d, J=7.6 Hz) 7.50-7.13 (4H, m) 7.25 (1H, d, J=16.8 Hz) 7.08-6.94 (2H, m) 4.64 (1H, septet, J=6.0 Hz) 1.30 (6H, d, J=6.0 Hz)

### Example 17

### 1-(4-Isopropoxyphenyl)-3-(2-pyridin-2-ylethyl)-5-(5-trifluoromethylpyridin-2-yl)-indole-2-carboxylic acid

The title compound was prepared in accordance with Example 15 from 3-iodo-1-(4-isopropoxyphenyl)-5-(5-trifluoromethylpyridin-2-yl)indole-2-carboxylic acid ethyl ester (Example 15, step (d)) and 2-vinylpiridine.

200 MHz ¹H-NMR (DMSO-d₆, ppm) δ 13.4-12.8 (1H, br s) 9.00 (1H, s) 8.55-8.49 (1H, m) 8.47-8.43 (1H, m) 8.28-8.15 (2H, m) 8.04 (1H, dd, J=8.9, 1.5 Hz) 7.66 (1H, ddd, J=7.5, 7.5, 1.9 Hz) 7.30-7.13 (4H, m) 7.09 (1H, d, J=8.9 Hz) 7.06-6.96 (2H, m) 4.66 (1H, septet, J=6.0 Hz) 3.63-3.44 (2H, m) 3.22-3.03 (2H, m) 1.31 (6H, d, J=6.0 Hz)

### Example 18

### 3-tert-Butylsulfanyl-1-(4-isopropoxyphenyl)-5-(5-trifluoromethylpyridin-2-yl)-indole-2-carboxylic acid

### (a) 3-tert-Butylsulfanyl-1-(4-isopropoxyphenyl)-5-(5-trifluoromethylpyridin-2-yl)indole-2-carboxylic acid ethyl ester

A solution of Pd₂(dba)₃ (9.2 mg, 0.01 mmol) and DPEphos (10.9 mg, 0.02 mmol) and tert-butylthiol (0.76 mL, 0.67 mmol) in toluene (3.3 mL) was added to a mixture of 3-iodo-1-(4-isopropoxyphenyl)-5-(5-trifluoromethylpyridin-2-yl)-indole-2-carboxylic acid ethyl ester (200 mg, 0.34 mmol, Example 15 step (d)) and potassium tert-butoxide (75.4 mg, 0.67 mmol). The mixture was stirred at 100° C. for 24 h and cooled to rt. The mixture was diluted with EtOAc and filtered through silica gel. The solids were washed with EtOAc and the combined filtrates were washed with NaHCO₃ (aq, sat) and brine, dried (Na₂SO₄), concentrated and purified by chromatography to afford the sub-title compound (170 mg, 90%).

### (b) 3-tert-Butylsulfanyl-1-(4-isopropoxyphenyl)-5-(5-trifluoromethylpyridin-2-yl)indole-2-carboxylic acid

The title compound was prepared in accordance with Example 15, step (g) from 3-tert-butylsulfanyl-1-(4-isopropoxyphenyl)-5-(5-trifluoromethylpyridin-2-yl)-indole-2-carboxylic acid ethyl ester (step (a) above).

200 MHz ¹H-NMR (DMSO-d₆, ppm) δ 13.4 (1H, br s) 9.03 (1H, s) 8.60 (1H, d, J=1.3 Hz) 8.28-8.16 (2H, m) 8.10 (1H, dd, J=8.8, 1.3 Hz) 7.40-7.30 (2H, m) 7.26 (1H, d, J=8.8 Hz) 7.12-7.02 (2H, m) 4.68 (1H, septet, J=6.0 Hz) 1.31 (6H, d, J=6.0 Hz) 1.28 (9H, s).

### Example 19

### 1-(4-Isopropoxyphenyl)-3-methyl-5-(5-trifluoromethylpyridin-2-yl)indole-2-carboxylic acid

### (a) 5-Bromo-3-methylindole-2-carboxylic acid ethyl ester

A solution of H₂SO₄ (cone, 1.76 g) in absolute EtOH (50 mL) was added to a suspension of 4-bromophenylhydrazine hydrochloride (6.57 g, 29.40 mmol) and 2-ketobutyric acid (3 g, 29.40 mmol) in EtOH (80 mL) and the mixture was heated at reflux for 4 h and kept at 4° C. for 14 h. The solid which formed was collected, washed with H₂O and dried to yield the sub-title compound (3.69 g, 44%).

### (b) 5-Bromo-1-(4-isopropoxyphenyl)-3-methylindole-2-carboxylic acid ethyl ester

The sub-title compound was prepared in accordance with Example 1 step (b) from 5-bromo-3-methylindole-2-carboxylic acid ethyl ester (see step (a) above) and 4-isopropoxyphenylboronic acid.

### (c) 1-(4-Isopropoxyphenyl)-3-methyl-5-(4,4,5,5-tetramethyl-[1,3,2]dioxaborolan-2-yl)indole-2-carboxylic acid ethyl ester

The sub-title compound was prepared in accordance with Example 9 step (a) from 5-bromo-1-(4-isopropoxyphenyl)-3-methylindole-2-carboxylic acid ethyl ester (see step (b) above) and bis(pinacolato)diboron.

### (d) 1-(4-Isopropoxyphenyl)-3-methyl-5-(5-trifluoromethylpyridin-2-yl)indole-2-carboxylic acid ethyl ester

The sub-title compound was prepared in accordance with Example 9 step (b) from 1-(4-isopropoxyphenyl)-3-methyl-5-(4,4,5,5-tetramethyl-[1,3,2]dioxaborolan-2-yl)indole-2-carboxylic acid ethyl ester (see step (c) above) and 2-bromo-5-(trifluoromethyl)pyridine.

### (e) 1-(4-Isopropoxyphenyl)-3-methyl-5-(5-trifluoromethylpyridin-2-yl)indole-2-carboxylic acid

The title compound was prepared in accordance with Example 2 step (b) from 1-(4-isopropoxyphenyl)-3-methyl-5-(5-trifluoromethylpyridin-2-yl)indole-2-carboxylic acid ethyl ester.

200 MHz ¹H-NMR (acetone -d₆, ppm) δ 9.03-8.94 (1H, m) 8.68-8.61 (1H, m) 8.31-8.11 (3H, m) 7.34-7.24 (2H, m) 7.16 (1H, d, J=8.8 Hz) 7.11-7.01 (2H, m) 4.71 (1H, septet, J=6.0 Hz) 2.76 (3H, s) 1.37 (6H, d, J=6.0 Hz).

### Example 20

### 3-Cyano-1-(4-isopropoxyphenyl)-5-(5-trifluoromethylpyrid carboxylic acid

### (a) 3-Cyano-1-(4-isopropoxyphenyl)-5-(5-trifluoromethylpyridin-2-yl)indole-2-carboxylic acid ethyl ester

A solution of hydroxylamine hydrochloride (365 mg, 5.24 mmol) and 3-formyl-1-(4-isopropoxyphenyl)-5-(5-trifluoromethylpyridin-2-yl)indole-2-carboxylic acid ethyl ester (see Example 9, step (b)) in formic acid (35 mL) was heated at reflux for 3.5 h. The mixture was allowed to cool and the pH was adjusted to 6 with NaOH (aq, 1 M). The mixture was extracted with EtOAc and the combined extracts washed with water and brine, dried (Na₂SO₄), concentrated and purified by chromatography to yield 1.73 g (87%) of sub-title product.

### (b) 3-Cyano-1-(4-isopropoxyphenyl)-5-(5-trifluoromethylpyridin-2-yl)indole-2-carboxylic acid

The title compound was prepared in accordance with Example 2 step (b) from 3-cyano-1-(4-isopropoxyphenyl)-5-(5-trifluoromethylpyridin-2-yl)indole-2-carboxylic acid ethyl ester.

200 MHz ¹H-NMR (DMSO-d₆, ppm) δ 14.5-13.5 (1H, br s) 9.10-9.04 (1H, m) 8.62 (1H, d, J=1.0 Hz) 8.37 (1H, d, J=8.4 Hz) 8.33-8.22 (2H, m) 7.47-7.37 (2H, m) 7.25 (1H, d, J=8.9 Hz) 7.14-7.04 (2H, m) 4.72 (1H, septet, J=6.0 Hz) 1.34 (6H, d, J=6.0 Hz)

### Example 21

### 2-Carboxy-1-(4-isopropoxyphenyl)-5-(5-trifluoromethylpyridin-2-yl)indol-3-yl-methyl]pyridin-2-ylmethyl ammonium dichloride

The title compound was prepared in accordance with Example 2 step (a) from 3-formyl-1-(4-isopropoxyphenyl)-5-(5-trifluoromethylpyridin-2-yl)indole-2-carboxylic acid ethyl ester (see Example 9, step (b)) and 2-(aminomethyl)pyridine, followed by hydrolysis (see Example 2, step (b)) and salt formation (see Example 5, step (b)).

¹H NMR (DMSO-d₆, 200 MHz): δ 14.0-13.0 (1H, br s) 9.7-9.3 (2H, br s) 9.08-9.03 (1H, m) 8.89-8.84 (1H, m) 8.68-8.62 (1H, m) 8.40-8.28 (2H, m) 8.21 (1H, dd, J=8.8, 1.2 Hz) 7.87 (1H, ddd, J=7.7 , 7.7, 1.7 Hz) 7.53 (1H, d, J=7.7 Hz) 7.43 (1H, dd, J=7.7, 5.1 Hz) 7.33-7.24 (2H, m) 7.16 (1H, d, J=8.8 Hz) 7.12-7.04 (2H, m) 4.86 (2H, s) 4.71 (1H, septet, J=6.0 Hz) 4.46 (2H, s) 1.34 (6H, d, J=6.0 Hz)

### Example 22

### 3-Acetyl-1-(4-isopropoxyphenyl)-5-(5-trifluoromethylpyridin-2-yl)indole-2-carboxylic acid

### (a) 3-Acetyl-5-bromoindole-2-carboxylic acid ethyl ester

Et₂AlCl (1 M in hexane, 14.9 mL, 14.9 mmol)) was added to a solution of 5-bromoindole-2-carboxylic acid ethyl ester (2.00 g, 7.46 mmol) in CH₂Cl₂ (40 mL) at 0° C. under argon. The mixture was stirred at 0° C. for 30 min and acetyl chloride (1.17 g, 14.92 mmol) in CH₂Cl₂ (40 mL) was added dropwise. The mixture was kept for 12 h at 4° C. and stirred at rt for 4 h. NaHCO₃ (aq, sat) was added and the mixture was extracted with EtOAc. The combined extracts were washed with water and brine, dried (Na₂SO₄), concentrated and purified by chromatography to yield 754 mg (33%) of the sub-title product.

### (b) 3-Acetyl-5-bromo-1-(4-isopropoxyphenyl)indole-2-carboxylic acid ethyl ester

The sub-title compound was prepared in accordance with Example 1 step (b) from 3-acetyl-5-bromoindole-2-carboxylic acid ethyl ester (see step (a) above) and 4-isopropoxyphenylboronic acid.

### (c) 3-Acetyl-1-(4-isopropoxyphenyl)-5-(4,4,5,5-tetramethyl-[1,3,2]dioxaborolan-2-yl)indole-2-carboxylic acid ethyl ester

The sub-title compound was prepared in accordance with Example 9 step (a) from 3-acetyl-5-bromo-1-(4-isopropoxyphenyl)-3-methylindole-2-carboxylic acid ethyl ester (see step (b) above) and bis(pinacolato)diboron.

### (d) 3-Acetyl-1-(4-isopropoxyphenyl)-5-(5-trifluoromethylpyridin-2-yl)indole-2-carboxylic acid ethyl ester

The sub-title compound was prepared in accordance with Example 9 step (b) from 3-acetyl-1-(4-isopropoxyphenyl)-3-methyl-5-(4,4,5,5-tetramethyl-[1,3,2]dioxaborolan-2-yl)indole-2-carboxylic acid ethyl ester (see step (c) above) and 2-bromo-5-(trifluoromethyl)pyridine.

### (e) 3-Acetyl-1-(4-isopropoxyphenyl)-5-(5-trifluoromethylpyridin-2-yl)indole-2-carboxylic acid

The title compound was prepared in accordance with Example 2 step (b) from 3-acetyl-1-(4-isopropoxyphenyl)-5-(5-trifluoromethylpyridin-2-yl)indole-2-carboxylic acid ethyl ester (see step (d) above).

200 MHz ¹H-NMR (DMSO-d₆, ppm) δ 4.6-13.9 (1H, br s) 9.09-9.04 (1H, m) 8.98 (1H, d, J=1.4 Hz) 8.32-8.19 (2H, m) 8.13 (1H, dd, J=8.8, 1.7 Hz) 7.46-7.37 (2H, m) 7.26 (1H, d, J=8.8 Hz) 7.18-7.08 (2H, m) 4.72 (1H, septet, J=6.0 Hz) 2.62 (3H, s) 1.33 (6H, d, J=6.0 Hz).

### Example 23

### 3-Ethyl-1-(4-isopropoxyphenyl)-5-(5-trifluoromethylpyridin-2-yl)indole-2-carboxylic acid

### (a) 5-Bromo-3-ethylindole-2-carboxylic acid ethyl ester

Et₃SiH (953 μL, 5.90 mmol) was added to a solution of 3-acetyl-5-bromoindole-2-carboxylic acid ethyl ester (see Example 22, step (a)) (477 mg. 1.54 mmol) in CF₃COOH (4 mL). The mixture was stirred at rt for 2.5 h, poured into water and extracted with EtOAc. The combined extracts were washed with water and brine, dried (Na₂SO₄) and concentrated. Crystallisation from EtOH gave the sub-title compound (300 mg, 66%.

### (b) 5-Bromo-3-ethyl-1-(4-isopropoxyphenyl)indole-2-carboxylic acid ethyl ester

The sub-title compound was prepared in accordance with Example 1 step (b) from 5-bromo-3-ethylindole-2-carboxylic acid ethyl ester (see step (a) above) and 4-isopropoxyphenylboronic acid.

### (c) 3-Ethyl-1-(4-isopropoxyphenyl)-5-(4,4,5,5-tetramethyl-[1,3,2]dioxaborolan-2-yl)indole-2-carboxylic acid ethyl ester

The sub-title compound was prepared in accordance with Example 9 step (a) from 5-bromo-3-ethyl-1-(4-isopropoxyphenyl)-3-methylindole-2-carboxylic acid ethyl ester (see step (b) above) and bis(pinacolato)diboron.

### (d) 3-Ethyl-1-(4-isopropoxyphenyl)-5-(5-trifluoromethylpyridin-2-yl)indole-2-carboxylic acid ethyl ester

The sub-title compound was prepared in accordance with Example 9 step (b) from 3-ethyl-1-(4-isopropoxyphenyl)-3-methyl-5-(4,4,5,5-tetramethyl-[1,3,2]dioxaborolan-2-yl)indole-2-carboxylic acid ethyl ester (see step (c) above) and 2-bromo-5-(trifluoromethyl)pyridine.

### (e) 3-Ethyl-1-(4-isopropoxyphenyl)-5-(5-trifluoromethylpyridin-2-yl)indole-2-carboxylic acid

The title compound was prepared in accordance with Example 2 step (b) from 3-ethyl-1-(4-isopropoxyphenyl)-5-(5-trifluoromethylpyridin-2-yl)indole-2-carboxylic acid ethyl ester (see step (d) above).

200 MHz ¹H-NMR (DMSO-d₆, ppm) δ 12.89 (1H, s) 9.05-9.00 (1H, m) 8.63-8.59 (1H, m) 8.34-8.21 (2H, m) 8.12 (1H, dd, J=8.8, 1.5 Hz) 7.29-7.21 (2H, m) 7.12 (1H, d, J=8.8 Hz) 7.08-6.99 (2H, m) 4.69 (1H, septet, J=6.0 Hz) 3.26-3.11 (2H, m) 1.33 (6H, d, J=6.0 Hz) 1.30 (3H, t, J=7.4 Hz).

### Example 24

### 1-(4-Isopropoxyphenyl)-3-methyl-5-(4-trifluoromethoxyphenyl)indole-2-carboxylic acid

### (a) 1-(4-Isopropoxyphenyl)-3-methyl-5-(4-trifluoromethoxyphenyl)indole-2-carboxylic acid ethyl ester

The sub-title compound was prepared in accordance with Example 1 step (c) from from 5-bromo-1-(4-isopropoxyphenyl)-3-methylindole-2-carboxylic acid ethyl ester (see Example 19, step (b)) and 4-trifluoromethoxyphenylboronic acid.

### (b) 1-(4-Isopropoxyphenyl)-3-methyl-5-(4-trifluoromethoxyphenyl)indole-2-carboxylic acid

The title compound was prepared in accordance with Example 2 step (b) from 1-(4-isopropoxyphenyl)-3-methyl-5-(4-trifluoromethoxyphenyl)indole-2-carboxylic acid ethyl ester (see step (a) above).

200 MHz ¹H-NMR (DMSO-d₆, ppm) δ 12.9-12.6 (1H, br s) 8.05-8.01 (1H, m) 7.88-7.78 (2H, m) 7.58 (1H, dd, J=8.8, 1.4 Hz) 7.49-7.40 (2H, m) 7.27-7.18 (2H, m) 7.10-6.98 (3H, m) 4.68 (1H, septet, J=6.0 Hz) 2.63 (3H, s) 1.33 (6H, d, J=6.0 Hz).

### Example 25

### 1-(4-Isopropoxyphenyl)-3-methylsulfanyl-5-(5-trifluoromethylpyridin-2-yl)-indole-2-carboxylic acid

### (a) 5-Bromo-3-iodoindole-2-carboxylic acid ethyl ester

A solution of NaI (6.66 g, 44.8 mmol) in acetone (170 mL) was added dropwise, over 15 min to a solution of N-chlorosuccinimide (6.0 g, 44.8 mmol) in acetone (70 mL). After stirring under argon for 15 min, a solution of 5-bromoindole-2-carboxylic acid ethyl ester (10.0 g, 37.3 mmol) in acetone (70 mL) was added dropwise. After stirring for 30 min at rt, the mixture was poured into Na₂S₂O₃ (aq, sat) and extracted with EtOAc (3×200 mL). The combined extracts were washed with water and brine, dried (Na₂SO₄) and concentrated. Crystallisation from EtOAc-petroleum ether gave the sub-title compound (13.5 g, 92%).

### (b) 5-Bromo-3-iodo-1-(4-isopropoxyphenyl)indole-2-carboxylic acid ethyl ester

The sub-title compound was prepared in accordance with Example 1 step (b) from 5-bromo-3-iodoindole-2-carboxylic acid ethyl ester (see step (a) above) and 4-isopropoxyphenylboronic acid.

### (c) 5-Bromo-1-(4-isopropoxyphenyl)-3-methylsulfanylindole-2-carboxylic acid ethyl ester

iPrMgCl.LiCl (1 M in THF, 5.0 mL, 5 mmol) was added at −40° C. to a solution of 5-bromo-3-iodo-1-(4-isopropoxyphenyl)indole-2-carboxylic acid ethyl ester (see step (b) above; 1.2 g, 2.27 mmol) in THF (10 mL). Me₂S₂ (1.0 mL, 11.35 mmol) was added after 30 min and the mixture was stirred at rt overnight. NH₄Cl (aq, sat) was added and the mixture was extracted with EtOAc (3×100 mL). The combined extracts were washed with water and brine, dried (Na₂SO₄), concentrated and purified by chromatography to afford the sub-title compound (1.15 g, 85%).

### (d) 1-(4-Isopropoxyphenyl)-3-methylsulfanyl-5-(5-trifluoromethylpyridin-2-yl)-indole-2-carboxylic acid ethyl ester hydrochloride

t-BuLi (1.5 M in pentane, 3.0 mL, 4.5 mmol) was added dropwise at −78° C. to Et₂O (10 mL). 2-Bromo-5-(trifluoromethyl)pyridine (504 mg, 2.23 mmol) in Et₂O (3.0 mL) was added via syringe and the mixture was stirred at −78° C. for 20 min and cannulated into ZnCl₂ (1 M in Et₂O, 4.9 mL, 4.9 mmol) cooled to −78° C. The mixture was allowed to warm to rt and was stirred for 3 h. THF (10 mL) was added and the solution was cannulated into a mixture of 5-bromo-1-(4-isopropoxyphenyl)-3-methylsulfanylindole-2-carboxylic acid ethyl ester (see step (c) above, 500 mg, 1.12 mmol), Pd(dppf)Cl₂ (109 mg, 0.13 mmol), CuI (51 mg, 0.27 mmol) and N-methyl-pyrrolidin-2-one (3.5 mL) under argon. The mixture was heated at 80° C. for 6 h, poured into NH₄Cl (aq, sat, 50 mL) and extracted with t-BuOMe (3×25 mL). The combined extracts were washed with brine, dried (Na₂SO₄) and filtered through Celite®. The solids were washed with t-BuOMe, and the combined filtrates were concentrated and dissolved in a dry Et₂O. HCl (4 M in dioxane, 500 μL, 2.0 mmol) was added and the mixture was stirred for 10 min and concentrated. Trituration with anhydrous Et₂O afforded the sub-title compound (200 mg, 32%).

### (e) 1-(4-Isopropoxyphenyl)-3-methylsulfanyl-5-(5-trifluoromethylpyridin-2-yl)-indole-2-carboxylic acid

A mixture of 1-(4-isopropoxyphenyl)-3-methylsulfanyl-5-(5-trifluoromethylpyridin-2-yl)indole-2-carboxylic acid ethyl ester hydrochloric salt (200 mg, 0.36 mmol, see step (d) above), NaOH (aq, 2 M, 2 mL) and dioxane (3 mL) was heated at 80° C. for 4 h. The mixture was acidified to pH 5 with HCl (aq, 1 M) and filtered. The solid was recrystallised from EtOAc to afford the title compound (98 mg, 56%).

200 MHz ¹H-NMR (DMSO-d₆, ppm) δ 13.4-13.3 (1H, br s) 9.04 (1H, s) 8.62 (1H, s) 8.27 (2H, m) 8.13 (1H, dd, J=8.7, 1.6 Hz) 7.35-7.27 (2H, m) 7.22 (1H, d, J=8.7 Hz) 7.09-7.00 (2H, m) 4.68 (1H, septet, J=6.0 Hz) 3.31 (3H, s, overlapped with water) 1.31 (6H, d, J=6.0 Hz).

### Example 26

### 1-(4-Isopropoxyphenyl)-3-methanesulfinyl-5-(5-trifluoromethylpyridin-2-yl)-indole-2-carboxylic acid

### (a) 5-Bromo-1-(4-isopropoxyphenyl)-3-methanesulfinylindole-2-carboxylic acid ethyl ester

A mixture of 5-bromo-1-(4-isopropoxyphenyl)-3-methylsulfanylindole-2-carboxylic acid ethyl ester (315 mg, 0.70 mmol; see Example 25, step (c)), tetrabutylammonium periodate (335 mg, 0.77 mmol) and 5,10,15,20-tetraphenyl-21H,23H-porphine iron (III) chloride (10 mg, 0.014 mmol) and CH₂Cl₂ was stirred at 0° C. for 6 h. Concentration and purification by chromatography afforded the sub-title compound (220 mg, 67%).

### (b) 1-(4-Isopropoxyphenyl)-3-methanesulfinyl-5-(5-trifluoromethylpyridin-2-yl)-indole-2-carboxylic acid

The title compound was prepared in accordance with Example 25, step (d) from 5-bromo-1-(4-isopropoxyphenyl)-3-methanesulfinylindole-2-carboxylic acid ethyl ester (see step (a) above), followed by hydrolysis (see Example 25, step (e)).

200 MHz ¹H-NMR (DMSO-d₆, ppm) δ 14.0-13.7 (1H, br s) 9.27 (1H, s) 9.04 (1H, s) 8.27 (1H, dd, J=9.0, 1.9 Hz) 8.19-8.10 (2H, m) 7.39-7.29 (2H, m) 7.18 (1H, d, J=9.0 Hz) 7.10-7.01 (2H, m) 4.69 (1H, septet, J=6.0 Hz) 3.07 (3H, s) 1.32 (6H, d, J=6.0 Hz).

### Example 27

### 1-(4-Isopropoxyphenyl)-3-methanesulfonyl-5-(5-trifluoromethylpyridin-2-yl)-indole-2-carboxylic acid

### (a) 5-Bromo-1-(4-isopropoxyphenyl)-3-methanesulfonylindole-2-carboxylic acid ethyl ester

Oxone® (2.16 g, 3.51 mmol) in water (9 mL) was added to a cooled solution of 5-bromo-1-(4-isopropoxyphenyl)-3-methylsulfanylindole-2-carboxylic acid ethyl ester (315 mg, 0.70 mmol; see Example 25, step (c)) in THF (6 mL) at 0° C. After stirring at rt for 4 days the mixture was extracted with EtOAc (3×50 mL). The combined extracts were washed with water, brine, dried (Na₂SO₄), concentrated and purified by chromatography to afford the sub-title compound (240 mg, 71%).

### (b) 1-(4-Isopropoxyphenyl)-3-methanesulfonyl-5-(5-trifluoromethylpyridin-2-yl)-(indole-2-carboxylic acid

The title compound was prepared in accordance with Example 25, step (d) from 5-bromo-1-(4-isopropoxyphenyl)-3-methanesulfonylindole-2-carboxylic acid ethyl ester (see step (a) above), followed by hydrolysis (see Example 25, step (e)).

200 MHz ¹H-NMR (DMSO-d₆, ppm) δ 14.7-14.0 (1H, br s) 9.07 (1H, s) 8.84 (1H, s) 8.30 (1H, dd, J=8.7, 1.8 Hz) 8.21 (1H, d, J=8.7 Hz) 8.16 (1H, dd, J=9.0, 1.8 Hz) 7.46-7.38 (2H, m) 7.31 (1H, d, J=9.0 Hz) 7.16-7.07 (2H, m) 4.71 (1H, septet, J=6.0 Hz) 3.40 (3H, s) 1.32 (6H, d, J=6.0 Hz).

### Example 28

### 1-(4-Isopropoxyphenyl)-3-trifluoromethyl-5-(5-trifluoromethylpyridin-2-yl)-indole-2-carboxylic acid

### (a) N-(4-Chloro-phenyl)-2,2-dimethylpropionamide

2,2-dimethylpropionyl chloride (6.3 mL, 51.0 mmol) was added dropwise to a mixture of 4-chlorophenylamine (5 g, 39.2 mmol), Et₃N (7.2 mL, 51.0 mmol) and anhydrous CH₂Cl₂ (35 mL) at 0° C. The mixture was stirred for 6 h at rt, washed with water, dried (Na₂SO₄) and concentrated. The residue was crystallised from EtOAc-petroleum ether to afford the sub-title compound (7.74 g, 93%).

### (b) N-[4-Chloro-2-(2,2,2-trifluoroacetyl)phenyl]-2,2-dimethylpropionamide

TMEDA (3.6 mL, 23.6 mmol) was added to a suspension of N-(4-chlorophenyl)-2,2-dimethylpropionamide (5 g, 23.6 mmol; see step (a) above) in anhydrous Et₂O (50 mL). The mixture was cooled to −15° C. and n-BuLi (2.5 M in hexanes, 22 mL, 54.3 mmol) was introduced via syringe. The mixture was kept at 0° C. for 2 h and cooled to −20° C. Trifluoroacetic acid methyl ester (3.33 mL, 33.1 mmol) was added rapidly. After 30 min, HCl (aq, 1 M, 150 mL) was added keeping the temperature below 25° C. The organic layer was collected and the aqueous layer was extracted with EtOAc. The combined organic phases were washed with water, brine, dried (Na₂SO₄), concentrated and purified by chromatography to give the sub-title compound (5.5 g, 75%).

### (c) 1-(2-Amino-5-chlorophenyl)-2,2,2-trifluoroethanone

HCl (aq, conc) was added to a solution of N-[4-Chloro-2-(2,2,2-trifluoroacetyl)-phenyl]-2,2-dimethyl-propionamide (5.5 g, 17.9 mmol; see step (b) above) in glacial acetic acid (50 mL) and the mixture was heated at 65-70° C. for 4 h. The slurry was cooled to 0-5° C. and the solid was filtered off, washed with petroleum ether/EtOAc (10:1) and dissolved in t-BuOMe (25 mL). Water (6.5 mL) and NaOAc (2.15 g, 32.9 mmol) were added and the mixture was stirred at rt for 30 min. The organic layer was collected, washed with water and brine, dried (Na₂SO₄) and concentrated The solid residue was recrystallised from EtOAc/petroleum ether to afford the sub-title compound (3.44 g, 86%).

### (d) N-[4-Chloro-2-(2,2,2-trifluoroacetyl)phenyl]oxalamic acid ethyl ester

A mixture of 1-(2-amino-5-chlorophenyl)-2,2,2-trifluoroethanone (3.72 g, 21.9 mmol; see step (c) above), chlorooxoacetic acid ethyl ester (2.45 mL, 21.9 mmol) and toluene (20 mL) was heated with stirring at 110° C. for 4 h. On cooling to −15° C., a precipitate was formed, which was filtered off, washed with petroleum ether and dried to afford 4.37 g (81%) of the sub-title compound.

### (e) 5-Chloro-3-trifluoromethylindole-2-carboxylic acid ethyl ester

A solution of TiCl₄ (3.6 mL, 32.8 mmol) in THF (120 mL) was added to N-[4-chloro-2-(2,2,2-trifluoroacetyl)phenyl]oxalamic acid ethyl ester (4.37 g, 13.5 mmol; see step (d) above) and zinc dust (4.24 g, 64.8 mmol) in THF (20 mL). After stirring for 2 h under argon, the mixture was absorbed on silica gel (100 mL) which was eluated with CH₂Cl₂ The eluent was concentrated and purified by chromatography affording the sub-title compound (2.0 g, 51%).

### (f) 5-Chloro-1-(4-isopropoxyphenyl)-3-trifluoromethylindole-2-carboxylic acid ethyl ester

The sub-title compound was prepared in accordance with Example 1, step (b) from 5-chloro-3-trifluoromethylindole-2-carboxylic acid ethyl ester (see step (e) above) and 4-isopropoxyphenylboronic acid.

### (g) 1-(4-Isopropoxyphenyl)-5-(4,4,5,5-tetramethyl[1,3,2]dioxaborolan-2-yl)-3-trifluoromethylindole-2-carboxylic acid ethyl ester

The sub-title compound was prepared in accordance with Example 9, step (a) from 5-chloro-1-(4-isopropoxyphenyl)-3-trifluoromethylindole-2-carboxylic acid ethyl ester (see step (f) above) and bis(pinacolato)diboron.

### (h) 1-(4-Isopropoxyphenyl)-3-trifluoromethyl-5-(5-trifluoromethylpyridin-2-yl)-indole-2-carboxylic acid ethyl ester

The sub-title compound was prepared in accordance with Example 9, step (b) from 1-(4-isopropoxyphenyl)-5-(4,4,5,5-tetramethyl-[1,3,2]dioxaborolan-2-yl)-3-trifluoromethylindole-2-carboxylic acid ethyl ester (see step (g) above) and 2-bromo-5-(trifluoromethyl)pyridine.

### (i) 1-(4-Isopropoxyphenyl)-3-trifluoromethyl-5-(5-trifluoromethylpyridin-2-yl)-indole-2-carboxylic acid

The title compound was prepared in accordance with Example 2, step (b) from 1-(4-isopropoxyphenyl)-3-trifluoromethyl-5-(5-trifluoromethylpyridin-2-yl)-indole-2-carboxylic acid ethyl ester (see step (h) above.

200 MHz ¹H-NMR (DMSO-d₆, ppm) δ 14.5-13.5 (1H, br s) 9.04 (1H, s) 8.58 (1H, s) 8.25-8.23 (2H, m) 8.15 (1H, dd, J=9.0, 1.6 Hz) 7.43-7.36 (2H, m) 7.27 (1H, d, J=9.0 Hz) 7.13-7.06 (2H, m) 4.69 (1H, septet, J=6.0 Hz) 1.31 (6H, d, J=6.0 Hz).

### Example 29

### 3-[5-(4-tert-Butylphenyl)-1-(4-cyclopentyloxyphenyl)indol-2-yl]-propionic acid

### (a) 5-(4-tert-Butylphenyl)indole-2-carboxylic acid ethyl ester

A mixture of 5-bromoindole-2-carboxylic acid ethyl ester (3.48 g, 13 mmol), 4-tert-butylphenylboronic acid (4.63 g, 26 mmol), K₃PO₄ (9.93 g, 45 mmol), Pd(OAc)₂ (146 mg, 0.65 mmol), tri-o-tolylphosphine (396 mg, 25 30 1.3 mmol), EtOH (20 ml) and toluene (10 mL) was stirred under argon for 20 min at rt followed by heating at 100° C. for 24 h. The mixture was allowed to cool to rt, poured into NaHCO₃ (aq, sat) and extracted with EtOAc. The combined extracts were washed with water and brine, dried (Na₂SO₄), concentrated and purified by chromatography to give the sub-title compound (3.27 g, 78%).

### (b) 5-(4-tert-Butylphenyl)-1-(4-cyclopentyloxyphenyl)indole-2-carboxylic acid ethyl ester

### Method A

A mixture of 5-(4-tert-butylphenyl)indole-2-carboxylic acid ethyl ester (0.95 g, 2.96 mmol; see step (a) above), CuI (56 mg, 0.30 mmol), K₃PO₄ (1.25 g, 5.90 mmol), N,N′-dimethyl-1,2-diaminoethane (91 μL, 0.89 mmol), 1-bromo-4-cyclopentyloxybenzene (1.42 g, 5:9 mmol) and toluene (10 mL) was heated at 110° C. for 24 h. The mixture was diluted with EtOAc and washed with NaHCO₃ (aq, sat), HCl (aq, 0.1 M) and brine and dried. (Na₂SO₄). Concentration and purification by chromatography gave the sub-title compound (1.96 g, 69%).

### Method B

Anhydrous CH₂Cl₂ (80 mL), followed by Et₃N (3.10 mL, 22.0 mmol) and pyridine (1.80 mL, 22.0 mmol) were added to 5-(4-tert-butylphenyl)indole-2-carboxylic acid ethyl ester (3.54 g, 11.0 mmol; see step (a) above), Cu(OAc)₂ (4.00 g, 22.0 mmol), 3 Å molecular sieves (ca. 7 g) and 4-cyclopentyloxyphenylboronic acid (4.54 g, 22.0 mmol). The mixture was stirred vigorously at rt for 48 h, then additional Et₃N (1.6 mL, 11.0 mmol), pyridine (0.90 mL, 11.0 mmol), Cu(OAc)₂ (2.00 g, 11.0 mmol) and 4-cyclopentyloxyphenylboronic acid (2.27 g, 11.0 mmol) was added, and the mixture was stirred at rt for 48 h. After the reaction was complete (as judged by TLC), the mixture was filtered through Celite® which was washed with EtOAc. The combined liquids were concentrated and purified by chromatography to afford the sub-title compound (3.7 g, 70%).

### (c) [5-(4-tert-Butylphenyl)-1-(4-cyclopentyloxyphenyl)indol-2-yl]-methanol

A solution of 5-(4-tert-butylphenyl)-1-(4-cyclopentyloxyphenyl)indole-2-carboxylic acid ethyl ester (1.93 g, 4.00 mmol; see step (b) above) in Et₂O (40 mL) was added dropwise under argon to a suspension of LiAlH₄ (300 mg, 8.0 mmol) in Et₂O (100 mL) at 0° C. The mixture was stirred at rt for 2 h, followed by addition of NH₄Cl (aq, sat) and EtOAc. The organic layer was collected and washed with NH₄Cl (aq, sat) and brine, dried (Na₂SO₄) and concentrated affording 1.67 g (95%) of the sub-title compound as a white solid.

### (d) 5-(4-tert-Butylphenyl)-1-(4-cyclopentyloxyphenyl)indole-2-carbaldehyde

To a solution of [5-(4-tert-butylphenyl)-1-(4-cyclopentyloxyphenyl)indol-2-yl]-methanol (0.53 g, 1.20 mmol; see step (c) above) in CH₂Cl₂ (10 mL) was added MnO₂ (350 mg, 4.03 mmol) at rt, and the mixture was stirred at rt for 24 h. Additional MnO₂ (350 mg, 4.03 mmol) was added, followed by two more portions (350 mg each) after 4. h and 8 h. After 20 h the mixture was filtered and concentrated. The solid residue was recrystallised from EtOH to yield 0.43 g (81%) of the sub-title compound.

### (e) 3-[5-(4-tert-Butylphenyl)-1-(4-cyclopentyloxyphenyl)indol-2-yl]acrylic acid ethyl ester

To a solution of 5-(4-tert-butylphenyl)-1-(4-cyclopentyloxyphenyl)indole-2-carbaldehyde (576 mg, 1.32 mmol; see step (d) above) in DMF (5 mL) was added (triphenylphosphoranylidene)acetic acid ethyl ester in DMF (2 mL). After 4 h at the mixture was poured into water (50 mL) and extracted with EtOAc (3×10 mL). The combined extracts were washed with water and brine, dried (Na₂SO₄), concentrated and purified by chromatography to give the sub-title compound (420 mg, 63%) as a yellow foam.

### (f) 3-[5-(4-tert-Butylphenyl)-1-(4-cyclopentyloxyphenyl)indol-2-yl]-propionic acid ethyl ester

A solution of 3-[5-(4-tert-butylphenyl)-1-(4-cyclopentyloxyphenyl)indol-2-yl]acrylic acid ethyl ester (225 mg, 1.32 mmol; see step (e) above) in a 1:1 mixture of EtOAc-EtOH (6 mL) was hydrogenated (rt, 5 atm) over 10% Pd on carbon (10 mg, 0.094 mmol) for 12 h. The mixture was filtered through a Celite®, concentrated and purified by chromatography affording the sub-title compound (210 mg, 95%) as a pale yellow oil.

### (g) 3-[5-(4-tert-Butylphenyl)-1-(4-cyclopentyloxyphenyl)indol-2-yl]propionic acid

The title compound was prepared in accordance with Example 2, step (b) from 3-[5-(4-tert-butylphenyl)-1-(4-cyclopentyloxyphenyl)indol-2-yl]-propionic acid ethyl ester (200 mg, 0.39 mmol; see step (f) above) in 59% yield (110 mg).

200 MHz ¹H-NMR (DMSO-d₆, ppm) δ 12.6-12.0 (1H, br s) 7.76-7.75 (1H, m) 7.59-7.53 (2H, m) 7.46-7.41 (2H, m) 7.34-7.28 (3H, m) 7.13-7.07 (2H, m) 6.97 (1H, d, J=8.6 Hz) 6.43 (1H, s) 4.94-4.86 (1H, m) 2.83-2.75 (2H, m) 2.60-2.53 (2H, m) 1.98-1.60 (8H, m) 1.30 (9H, s).

### Example 30

### 1-(4-Cyclopentyloxyphenyl)-2-(tetrazol-5-yl)-5-(5-trifluoromethylpyridin-2-yl)-indole

### (a) 5-Bromoindole-2-carboxylic acid amide

DMF (1.0 mL) and SOCl₂ (12.5 mL, 168 mmol) were added to a solution of 5-bromoindole-2-carboxylic acid (8.06 g, 34 mmol) in Et₂O (200 mL). After 2 h at rt, the volatiles were removed and the residue dissolved in Et₂O (200 mL) and added to NH₃ (1) at −60° C. The mixture was slowly allowed to warm to rt and was stirred for 12 h. The mixture was diluted with EtOAc (200 ml) and washed with water, NaHCO₃ (aq, sat), water and brine, dried (Na₂SO₄) and concentrated to give the sub-title compound (7.22 g, 90%), which was employed in the subsequent step without further purification.

### (b) 5-Bromoindole-2-carbonitrile

A solution of 5-bromoindole-2-carboxylic acid amide (7.22 g, 30 mmol; see step (a) above) and POCl₃ (160 mL) was heated under reflux for 15 min. The mixture was allowed to cool to rt, slowly poured into a mixture of crushed ice and cold aqueous NaOH and extracted with EtOAc. The combined extracts were washed with water and brine, dried (Na₂SO₄) and concentrated to give the sub-title compound (6.27 g, 94%), which was employed in the subsequent step without further purification.

### (c) 5-Bromo-1-(4-cyclopentyloxyphenyl)indole-2-carbonitrile

Anhydrous CH₂Cl₂ (270 mL), Et₃N (4.86 mL, 34.7 mmol) and pyridine (2.82 mL, 34.7 mmol) were added to 5-bromoindole-2-carbonitrile (3.83 g, 17.3 mmol; see step (b) above), Cu(OAc)₂ (6.29 g, 34.7 mmol), 3 Å molecular sieves (ca. 7 g) and 4-cyclopentyloxyphenylboronic acid (7.15 g, 34.7 mmol). The mixture was stirred vigorously at rt for 72 h and filtered through Celite®. The solids were washed with EtOAc, and the combined filtrates concentrated and purified by chromatography to afford the sub-title compound (3.87 g, 59%).

### (d) 1-(4-Cyclopentyloxyphenyl)-5-(4,4,5,5-tetramethyl-[1,3,2]dioxaborolan-2-yl)-indole-2-carbonitrile

Pd₂(dba)₃ (62 mg, 0.067 mmol) and tricyclohexylphosphine (113 mg, 0.40 mmol) in dioxane (13.5 mL) were added under argon to a stirred mixture of 5-bromo-1-(4-cyclopentyloxyphenyl)indole-2-carbonitrile (0.80 g, 2.1 mmol, see step (c) above), KOAc (0.30 g, 3.15 mmol), bis(pinacolato)diboron (0.59 g, 2.3 mmol) and dioxane (8 mL) at 80° C. After heating at 80° C. for 18 h, the mixture was allowed to cool and filtered through Celite®. The solids were washed with EtOAc and the combined filtrates were concentrated and purified by chromatography to yield the sub-title compound (0.55 g, 61%).

### (e) 1-(4-Cyclopentyloxyphenyl)-5-(5-trifluoromethylpyridin-2-yl)indole-2-carbonitrile

A stirred mixture of 1-(4-cyclopentyloxyphenyl)-5-(4,4,5,5-tetramethyl-[1,3,2]-dioxaborolan-2-yl)indole-2-carbonitrile (480 mg, 1.14 mmol; see step (d) above), 2-bromo-5-(trifluoromethyl)pyridine (380 mg, 1.72 mmol), Na₂CO₃ (aq, 2 M, 1.70 mL, 3.42 mmol), Pd(PPh₃)₄ (64 mg, 0.06 mmol), EtOH (5 mL) and toluene (20 mL) was heated at 80° C. for 23 h. The mixture was diluted with EtOAc, washed with brine, dried (Na₂SO₄), concentrated and purified by chromatography to give the sub-title compound (464 mg, 92%).

### (f) 1-(4-Cyclopentyloxyphenyl)-2-(tetrazol-5-yl)-5-(5-trifluoromethylpyridin-2-yl)indole

A stirred mixture of 1-(4-cyclopentyloxyphenyl)-5-(5-trifluoromethylpyridin-2-yl)indole-2-carbonitrile (147 mg, 0.33 mmol; see step (e) above), triethylammonium hydrochloride (136 mg, 0.99 mmol), sodium azide (64 mg, 0.99 mmol) and toluene (2 mL) was heated at 90° C. for 18 h. The mixture was diluted with EtOAc, washed with HCl (aq, 0.05 M) and brine, dried (Na₂SO₄), concentrated and purified by chromatography to give the title compound (143 mg, 88%).

200 MHz ¹H-NMR (DMSO-d₆, ppm) δ 9.01 (1H, s) 8.63 (1H, d, J=1.3 Hz) 8.30-8.20 (2H, m) 8.11 (1H, dd, J=8.8, 1.6 Hz) 7.45 (1H, s) 7.34-7.24 (2H, m) 7.23 (1H, d, J=8.8 Hz) 7.07-6.98 (2H, m) 4.94-4.82 (1H, m) 2.06-1.49 (8H, m).

### Example 31

### [5-(3-Chlorophenoxy)-1-(4-isopropoxyphenyl)indol-2-yl]acetic acid, triethylammonium salt

### (a) 2-Ethoxycarbonylmethyl-5-hydroxy-1-(4-isopropoxyphenyl)indole-3-carboxylic acid ethyl ester

To a solution of benzoquinone (1.41 g, 13.1 mmol) in MeCN (25 mL) was added 3-(4-isopropoxyamino)-3-ethoxycarbonylmethylacrylic acid ethyl ester (2.76 g, 10.5 mmol, prepared according to the procedure in J. Org. Chem. 16, 896 (1951). The mixture was heated under argon at 70° C. for 20 h and kept at 4° C. for 20 h. The solid was filtered off and recrystallised from MeCN to give the sub-title product (1.40 g, 40%).

### (b) 2-Carboxymethyl-5-hydroxy-1-(4-isopropoxyphenyl)indole-3-carboxylic acid

A mixture of 2-ethoxycarbonylmethyl-5-hydroxy-1-(4-isopropoxyphenyl)indole-3-carboxylic acid ethyl ester (0.40 g, 0.94 mmol; see step (a) above), NaOH (0.40 g, 10 mmol) and water (10 mL) was heated at reflux for 1 h and cooled to rt. Acidification with HCl (aq, conc) gave a precipitate which was filtered off, washed with water and dried to give the sub-title compound (0.34 g, 93%).

### (c) 2-Ethoxycarbonylmethyl-5-hydroxy-1-(4-isopropoxyphenyl)indole-3-carboxylic acid

A solution of 2-carboxymethyl-5-hydroxy-1-(4-isopropoxyphenyl)indole-3-carboxylic acid (330 mg, 0.89 mmol; see step (b) above) in HCl (0.5% in EtOH, 5 mL) was heated at reflux for 20 min. The mixture was concentrated and Na₂CO₃ (aq, 5%, 20 mL) was added. The mixture was washed with EtOAc and acidified with HCl (aq, conc) to give a precipitate which was filtered off, washed with water and dried to give the sub-title compound (207 mg, 58%).

### (d) [5-Hydroxy-1-(4-isopropoxyphenyl)indol-2-yl]acetic acid ethyl ester

2-Ethoxycarbonylmethyl-5-hydroxy-1-(4-isopropoxyphenyl)indole-3-carboxylic acid (200 mg, 0.5 mmol; see step (c) above) was heated at 230° C. under argon until the gas evolution ceased. Purification by chromatography gave the sub-title compound (113 mg, 63%) as on oil which solidified on standing.

### (e) [1-(4-Isopropoxyphenyl)-5-(3-chlorophenoxy)indol-2-yl]acetic acid ethyl ester

The sub-title compound was prepared from [5-hydroxy-1-(4-isopropoxyphenyl)-indol-2-yl]acetic acid ethyl ester (100 mg, 0.28 mmol; see step (d) above), 3-chlorophenylboronic acid (97 mg, 0.62 mmol), CH₂Cl₂, Et₃N, pyridine and Cu(OAc)₂ (see Example 30, step (c)) to afford the title compound in 49% yield. The product was used in the subsequent steps without further purification.

### (f) [5-(3-Chlorophenoxy)-1-(4-isopropoxyphenyl)indol-2-yl]acetic acid triethylammonium salt

A mixture of [1-(4-isopropoxyphenyl)-5-(3-chlorophenoxy)indol-2-yl]acetic acid ethyl ester (120 mg, 0.26 mmol; see step (e) above), LiOH monohydrate (140 mg, 3.34 mmol) and water (9 mL) was heated at reflux for 1.5 h, cooled to rt, acidified with citric acid (aq) and extracted with Et₂O. The combined extracts were dried (Na₂SO₄) and triethylamine was added. Concentration gave the title compound (105 mg, 75%) as a white foam.

200 MHz ¹H-NMR (DMSO-d₆, ppm) δ 7.30 (2H, d, J=8.8 Hz) 7.40-7.25 (4H, m) 7.13-7.03 (3H, m) 6.99 (1H, d, J=8.8 Hz) 6.95-6.85 (2H, m) 6.82 (1H, dd, J=8.6, 2.2 Hz) 6.51 (1H, s) 4.69 (1H, septet, J=6.0 Hz) 3.59 (2H, s, overlapped with water) 1.32 (6H, d, J=6.0 Hz).

### Example 32

### [5-(4-Chlorophenoxy)-1-(4-isopropoxyphenyl)indol-2-yl]acetic acid

The title compound was prepared in accordance with steps (e) and (0 in Example 31 from [5-hydroxy-1-(4-isopropoxyphenyl)indol-2-yl]acetic acid ethyl ester (see step (d) in Example 31) and 4-chlorophenylboronic acid, followed by hydrolysis and triethylamine salt formation, as described above.

200 MHz ¹H-NMR (DMSO-d₆, ppm) δ 7.39-7.29 (4H, m) 7.22 (1H, d, J=2.1 Hz) 7.10-6.88 (5H, m) 6.76 (1H, dd, J=8.8, 2.2 Hz) 6.42 (1H, s) 4.67 (1H, septet, J=6.0 Hz) 3.44 (2H, s) 1.31 (6H, d, J=6.0 Hz).

### Example 33

### [5-(2-Chlorophenoxy)-1-(4-isopropoxyphenyl)indol-2-yl]acetic acid

The title compound was prepared in accordance with steps (e) and (f) in Example 31 from [5-hydroxy-1-(4-isopropoxyphenyl)indol-2-yl]acetic acid ethyl ester (see step (d) in Example 31) and 2-chlorophenylboronic acid, followed by hydrolysis and triethylamine salt formation, as described above.

200 MHz ¹H-NMR (DMSO-d₆, ppm) δ 7.55 (1H, dd, J=8.0, 1.6 Hz) 7.35-7.21 (3H, m) 7.18 (1H, d, J=2.0 Hz) 7.15-7.03 (3H, m) 6.97 (1H, d, J=8.9 Hz) 6.88 (2H, dd, J=8.0, 1.3) 6.79 (1H, dd, J=8.7, 2.3 Hz) 6.48 (1H, s) 4.69 (1H, septet, J=6.0 Hz) 3.59 (2H, s) 1.32 (6H, d, J=6.0 Hz).

### Example 34

### 3-Chloro-1-(4-cyclopentyloxyphenyl)-2-(tetrazol-5-yl)-5-(5-trifluoromethylpyridin-2-yl)indole

### (a) 5-Bromo-3-chloroindole-2-carboxylic acid ethyl ester

A mixture of 5-bromoindole-2-carboxylic acid ethyl ester (4.00 g, 14.9 mmol), SO₂Cl₂ (1.8 mL, 22.4 mmol) and benzene (125 mL) was stirred at 90° C. for 2.5 h and cooled to rt. NaHCO₃ (aq, sat) was added and the mixture was extracted with EtOAc. The combined extracts were washed with water and brine, dried (Na₂SO₄) and concentrated. The residue was crystallised from toluene to yield the sub-title compound (3.87 g 85%).

### (b) 5-Bromo-3-chloroindole-2-'carboxylic acid

A mixture of 5-bromo-3-chloroindole-2-carboxylic acid ethyl ester (7.78 g, 25.7 mmol, see step (a) above), NaOH (5.14 g, 128 mmol), water (12 mL) and dioxane (50 mL) was stirred at 80° C. for 1 h and cooled to rt. HCl (1 M, 400 mL) was slowly added and the precipitate was collected and washed with water to give the sub-title compound (6.71 g 95%).

### (c) 5-Bromo-3-chloroindole-2-carbonitrile

The sub-title compound was prepared in accordance with steps (a) and (b) in Example 30 from 5-bromo-3-chloroindole-2-carboxylic acid (step (b) above).

### (d) 5-Bromo-3-chloro-1-(4-cyclopentyloxyphenyl)indole-2-carbonitrile

The sub-title compound was prepared in accordance with step (c) in Example 30 from 5-bromo-3-chloroindole-2-carbonitrile (step (c) above).

### (e) 3-Chloro-1-(4-cyclopentyloxyphenyl)-2-(tetrazol-5-yl)-5-(5-trifluoromethylpyridin-2-yl)indole

The title compound was prepared in accordance with steps (d), (e) and (f) in Example 30 from 5-bromo-3-chloro-1-(4-cyclopentyloxyphenyl)indole-2-carbonitrile (step (d) above).

200 MHz ¹H-NMR (DMSO-d₆, ppm) δ 9.05 (1H, s) 8.54 (1H, s) 8.22 (1H, d, J=8.6 Hz) 8.26 (1H, dd, J=8.6, 1.7 Hz) 8.02 (1H, dd, J=8.9, 1.6 Hz) 7.34 (1H, d, J=8.9 Hz) 7.30-7.21 (2H, m) 7.03-6.93 (2H, m) 4.90-4.78 (1H, m) 2.02-1.50 (8H, m)

### Example 35

### 1-(4-Cyclopentyloxyphenyl)-2-(tetrazol-5-yl)-5-(4-trifluoromethylphenyl)indole

A mixture of 5-bromo-1-(4-cyclopentyloxyphenyl)indole-2-carbonitrile (see step (c) in Example 30) (5.0 g, 13 mmol), 4-trifluorobenzeneboronic acid (4.94 g, 26 mmol), K₃PO₄ (9.93 g, 45 mmol), Pd(OAc)₂ (146 mg, 0.65 mmol), tri-o-tolylphosphine (396 mg, 1.3 mmol), EtOH (20 mL) and toluene (10 mL) was stirred under argon for 20 min at rt and heated at 100° C. for 24 h. The mixture was allowed to cool to rt, poured into NaHCO₃ (aq, sat) and extracted with EtOAc. The combined extracts were washed with water and brine and dried (Na₂SO₄). The tetrazole was subsequently prepared in accordance with step (f) in Example 30.

200 MHz ¹H-NMR (DMSO-d₆, ppm) δ 8.17 (1H, d, J=1.3 Hz) 8.00-7.89 (2H, m) 7.87-7.79 (2H, m) 7.65 (1H, dd, J=8.8, 1.3 Hz) 7.42 (1H, s) 7.34-7.25 (2H, m) 7.23 (1H, d, J=8.8 Hz) 7.09-6.99 (2H, m) 4.93-4.87 (1H, m) 2.11-1.51 (8H, m)

### Example 36

### 1-(4-Cyclopentyloxyphenyl)-2-(1H-tetrazol-5-yl)-5-(4-trifluoromethoxyphenyl)indole

The title compound was prepared in accordance with Example 34 from 5-bromo-1-(4-cyclopentyloxyphenyl)indole-2-carbonitrile (see step (c) in Example 30) and 4-trifluoromethoxybenzeneboronic acid.

200 MHz ¹H-NMR (DMSO-d₆, ppm) δ 8.08 (1H, d, J=1.3 Hz) 7.89-7.88 (2H, m) 7.59 (1H, dd, J=8.8, 1.3 Hz) 7.52-7.41 (2H, m) 7.40 (1H, s) 7.34-7.24 (2H, m) 7.21 (1H, d, J=8.8 Hz) 7.08-6.98 (2H, m) 4.95-4.83 (1H, m) 2.10-1.51 (8H, m)

### Example 37

### 3-Chloro-1-(4-cyclopentyloxyphenyl)-2-(tetrazol-5-yl)-5-(4-trifluoromethoxyphenyl)indole

The title compound was prepared in accordance with step (e) in Example 30 from 5-bromo-3-chloro-1-(4-cyclopentyloxyphenyl)indole-2-carbonitrile (see step (d) in Example 34) and 4-trifluoromethoxybenzeneboronic acid, followed by tetrazole formation in accordance with step (f) in Example 30.

200 MHz ¹H-NMR (DMSO-d₆, ppm) δ 7.93 (1H, s) 7.92-7.80 (2H, m) 7.67 (1H, d, J=8.9 Hz) 7.51-7.40 (2H, m) 7.28 (1H, d, J=8.9 Hz) 7.30-7.17 (2H, m) 7.02-6.90 (2H, m) 4.89-4.77 (1H, m) 2.04-1.44 (8H, m)

### Example 38

### 3-Chloro-1-(4-isopropoxyphenyl)-2-(tetrazol-5-yl)-5-(4-trifluoromethoxyphenyl)-indole

The title compound was prepared in accordance with Example 37 from 5-bromo-3-chloroindole-2-carbonitrile (see step (c) in Example 34), 4-isopropoxybenzene-boronic acid and 4-trifluoromethoxybenzeneboronic acid.

200 MHz ¹H-NMR (DMSO-d₆, ppm) δ 7.98-7.82 (3H, m) 7.67 (1H, dd, J=8.8, 1.6 Hz) 7.52-7.42 (2H, m) 7.30 (1H, d, J=8.8 Hz) 7.29-7.19 (2H, m) 7.05-6.94 (2H, m) 4.66 (1H, septet, J=6.0 Hz) 1.30 (6H, d, J=6.0 Hz)

### Example 39

### 3-Chloro-1-(4-cyclopropoxyphenyl)-2-(tetrazol-5-yl)-5-(4-trifluoromethoxyphenyl)indole

### (a) 1-Bromo-4-(2-bromoethoxy)benzene

A mixture of 4-bromophenol (30 g, 173 mmol), dibromoethane (40 mL, 464 mmol), NaOH (11.0 g, 275 mmol) and water (430 mL) was heated at reflux for 11 h. The layers were separated and the organic phase was concentrated and distilled to afford the sub-title compound (40.1 g 83%).

### (b) 1-Bromo-4-vinyloxybenzene

KOt-Bu (14.0 g, 125 mmol) was added in portions over 10 min to a solution of 1-bromo-4-(2-bromoethoxy)benzene (19.9 g, 100 mmol see step (a) above) in THF (120 mL) at 0° C. After 16 h at rt and dilution with water (400 mL), the mixture was extracted with petroleum ether (4×100 mL). The combined extracts were washed with brine, dried (Na₂SO₄) and concentrated. Vacuum distillation afforded the sub-title compound (11.5 g, 58%).

### (c) 1-Bromo-4-cyclopropoxybenzene

Diethylzinc (15% in hexanes, 95.5 mL, 116 mmol) was added to a mixture of 1-bromo-4-vinyloxybenzene (11.5 g, 58 mmol), chloroiodomethane (41 g, 232 mmol) and dichloroethane (180 mL) over 3 h at 0° C. After 30 min, NH₄Cl (aq, sat, 200 mL) and petroleum ether (300 mL) were added. The organic phase was collected and concentrated. The residue was dissolved in petroleum ether, filtered and concentrated to afford the sub-title compound (11.7 g, 94%).

### (d) 4-Cyclopropoxybenzene boronic acid

n-BuLi (2.5 M in hexane, 9.76 mL, 24.4 mmol) was added over 17 min to a solution of 1-bromo-4-cyclopropoxybenzene (5.0 g, 23.4 mmol) in THF (80 mL) at −78° C. After 40 min, B(OEt)₃ (5.9 mL, 34.3 mmol) was added and the mixture was allowed to reach rt and was stirred at rt for 18 h. The mixture was cooled to 0° C. and HCl (aq, 1 M, 70 mL) was added. After 30 min the mixture was extracted with t-BuOMe (3×50 mL) and the combined extracts were washed with brine, dried (Na₂SO₄) and concentrated. The residue was washed with petroleum ether to yield the sub-title compound (1.5 g, 34%).

### (e) 3-Chloro-1-(4-cyclopropoxyphenyl)-2-(tetrazol-5-yl)-5-(4-trifluoromethoxyphenyl)indole

The title compound was prepared in accordance with Example 37 from 5-bromo-3-chloroindole-2-carbonitrile (see step (c) in Example 34), 4-cyclopropoxybenzeneboronic acid (see step (d) above) and 4-trifluoromethoxybenzeneboronic acid.

200 MHz ¹H-NMR (DMSO-d₆, ppm) δ 7.97-7.83 (3H, m) 7.66 (1H, dd, J=8.8, 1.6 Hz) 7.53-7.42 (2H, m) 7.34-7.23 (3H, m) 7.20-7.10 (2H, m) 3.94-3.86 (1H, m) 0.88-0.64 (4H, m)

### Example 40

### 3-Chloro-1-(4-isopropoxyphenyl)-2-(tetrazol-5-yl)-5-(4-trifluoromethylphenyl)-indole

The title compound was prepared in accordance with Example 37 from 5-bromo-3-chloroindole-2-carbonitrile (see step (c) in Example 34), 4-isopropoxybenzeneboronic acid and 4-trifluoromethoxybenzeneboronic acid.

200 MHz ¹H-NMR (DMSO-d₆, ppm) δ 8.08-7.93 (3H, m) 7.89-7.79 (2H, m) 7.74 (1H, dd, J=8.8, 1.4 Hz) 7.34 (1H, d, J=8.8 Hz) 7.30-7.20 (2H, m) 7.06-6.95 (2H, m) 4.66 (1H, septet, J=6.0 Hz) 1.30 (6H, d, J=6.0 Hz)

### Example 41

### 1-(4-Isopropoxyphenyl)-2-(tetrazol-5-yl)-5-(4-trifluoromethylphenoxy)indole

### (a) 5-Benzyloxy-1-(4-isopropoxyphenyl)indole-2-carboxylic acid ethyl ester

5-Benzyloxyindole-2-carboxylic acid ethyl ester (2.38 g, 8.1 mmol), CuI (153 mg, 0.81 mmol), K₃PO₄ (3.43 g, 16.2 mmol), N,N′-dimethyl-1,2-diaminoethane (260 pt, 2.42 mmol) and 1-bromo-4-isopropoxybenzene (3.48 g, 16.2 mmol) in toluene (30 mL) were heated at 120° C. for 24 h. The mixture was diluted with EtOAc and washed with NaHCO₃ (aq, sat), HCl (aq, 0.1 M) and brine and dried (Na₂SO₄), concentrated and purified by chromatography to give the sub-title compound (2.99 g, 89%).

### (b) 5-Hydroxy-1-(4-isopropoxyphenyl)indole-2-carboxylic acid ethyl ester

A solution of 5-benzyloxy-1-(4-isopropoxyphenyl)indole-2-carboxylic acid ethyl ester (2.97 g, 6.9 mmol; see step (a) above) in EtOAc (60 mL) and EtOH (40 mL) was hydrogenated for 1 h at ambient temperature and pressure over Pd—C. Filtration through Celite® and concentration gave the sub-title compound (2.33 g, 99%).

### (c) 1-(4-Isopropoxyphenyl)-5-(4-trifluoromethylphenoxy)indole-2-carboxylic acid ethyl ester

Anhydrous CH₂Cl₂ (15 mL), Et₃N (0.40 mL, 2.94 mmol) and pyridine (0.23 g, 2.94 mmol) were added to 5-hydroxy-1-(4-isopropoxyphenyl)indole-2-carboxylic acid ethyl ester (0.50 g, 1.47 mmol; see step (b) above), Cu(OAc)₂ (0.27 g, 1.47 mmol) and trifluorobenzeneboronic acid (0.56 g, 2.94 mmol). The mixture was stirred vigorously at rt for 72 h. After the reaction was complete (as judged by TLC), the mixture was filtered through Celite®, concentrated and purified by chromatography to afford the sub-title compound (0.32 g, 55%).

### (d) 1-(4-Isopropoxyphenyl)-5-(4-trifluoromethylphenoxy)indole-2-carbonitrile

The sub-title compound was prepared in accordance with step (b) and (c) in Example 34 from 1-(4-isopropoxyphenyl)-5-(4-trifluoromethylphenoxy)indole-2-carboxylic acid ethyl ester (step (c) above).

### (e) 1-(4-Isopropoxyphenyl)-2-(tetrazol-5-yl)-5-(4-trifluoromethylphenoxy)indole

The title compound was prepared in accordance with step (1) in Example 30 from 1-(4-isopropoxyphenyl)-5-(4-trifluoromethylphenoxy)indole-2-carbonitrile (see step (d) above).

200 MHz ¹H-NMR (DMSO-d₆, ppm) δ 7.78-7.65 (2H, m) 7.59 (1H, d, J=1.8 Hz) 7.39-7.24 (3H, m) 7.23-6.98 (6H, m) 4.70 (1H, septet, J=6.1 Hz) 1.33 (6H, d, J=6.1 Hz)

### Example 42

### 3-Chloro-1-(4-isopropoxyphenyl)-2-(tetrazol-5-yl)-5-(4-trifluoromethylphenoxy)-indole

### (a) 3-Chloro-1-(4-isopropoxyphenyl)-5-(4-trifluoromethylphenoxy)indole-2-carboxylic acid ethyl ester

A solution of SO₂Cl₂ (243 μL, 3.90 mmol) in anhydrous Et₂O (20 mL) was added to solution of 1-(4-isopropoxyphenyl)-5-(4-trifluoromethylphenoxy)indole-2-carboxylic acid ethyl ester (0.967 g, 2.0 mmol, see Example 41, step (c)) in anhydrous Et₂O (75 mL) over 10 min at −9° C. The mixture was stirred at 0° C. for 24 h, washed with NaHCO₃ (aq, sat), water and brine, dried (Na₂SO₄) and concentrated. The residue was washed with a small amount of petroleum ether to give the sub-title compound (0.85 g, 82%).

### (b) 3-Chloro-1-(4-isopropoxyphenyl)-2-(tetrazol-5-yl)-5-(4-trifluoromethylphenoxy)indole

The title compound was prepared in accordance with steps (d) and (e) in Example 41 from 3-chloro-1-(4-isopropoxyphenyl)-5-(4-trifluoromethylphenoxy)indole-2-carboxylic acid ethyl ester (see step (a) above).

200 MHz ¹H-NMR (DMSO-d₆, ppm) S 7.80-7.67 (2H, m) 7.45 (1H, d, J=1.8 Hz) 7.36-7.10 (6H, m) 7.07-6.95 (2H, m) 4.66 (1H, septet, J=6.0 Hz) 1.29 (6H, d, J=6.0 Hz).

### Example 43

### 3-[5-(4-tert-Butyl-phenyl)-1-(4-cyclopentyloxyphenyl)indol-2-yl]acrylic acid

The title compound was prepared in accordance with Example 29, step (g) from 3-[5-(4-tert-butylphenyl)-1-(4-cyclopentyloxyphenyl)indol-2-yl]-acrylic acid ethyl ester (see Example 29, step (e)).

200 MHz ¹H-NMR (DMSO-d₆, ppm) δ 7.83 (1H, d, J=1.3 Hz) 7.61-7.55 (2H, m) 7.47-7.38 (3H, m) 7.33-7.26 (2H, m) 7.13-6.99 (5H, m) 6.37 (1H, d, J=16.0 Hz) 4.94-4.86 (1H, m) 1.99-1.59 (8H, m) 1.30 (9H, s).

### Example 44

### ((5-(4-tert-Butylphenyl)-1-(4-cyclopentyloxyphenyl)indol-2-ylmethyl)methylamino)acetic acid

### (a) ((5-(4-tert-Butylphenyl)-1-(4-cyclopentyloxyphenyl)indol-2-ylmethyl)methylamino)acetic acid ethyl ester

A mixture of 5-(4-tert-butylphenyl)-1-(4-cyclopentyloxyphenyl)indole-2-carbaldehyde (200 mg, 0.46 mmol; see Example 29, step (d)), N-methyl glycine ethyl ester hydrochloride (138 mg, 0.90 mmol), sodium acetate (52 mg, 0.72 mmol) and methanol (11 mL) was stirred for 1 h at rt. NaCNBH₃ (93 mg, 1.48 mmol) was added and the mixture was stirred at rt for a 24 h, poured into water and extracted with EtOAc. The combined extracts were washed with water and brine, dried (Na₂SO₄), concentrated and purified by chromatography to give the sub-title compound (120 mg, 49%).

### (b) ((5-(4-tert-Butylphenyl)-1-(4-cyclopentyloxyphenyl)indol-2-ylmethyl)methylamino)acetic acid

The title compound was prepared in accordance with Example 29, step (g) from ((5-(4-tert-butylphenyl)-1-(4-cyclopentyloxyphenyl)indol-2-ylmethyl)methylamino)acetic acid ethyl ester (see step (a) above).

200 MHz ¹H-NMR (DMSO-d₆, ppm) δ 12.5-11.5 (1H, br s) 7.82-7.77 (1H, m) 7.60-7.52 (2H, m) 7.47-7.29 (5H, m) 7.08-7.00 (3H, m) 6.59-6.56 (1H, m) 4.94-4.82 (1H, m) 3.67 (2H, s) 3.13 (2H, s) 2.56 (3H, s) 2.05-1.52 (8H, m) 1.29 (9H, s).

### Example 45

### 3-[3-Chloro-1-(4-isopropoxyphenyl)-5-(5-trifluoromethylpyridin-2-yl)indol-2-yl]-acrylic acid

### (a) 3-Chloro-5-(4,4,5,5-tetramethyl-[1,3,2]dioxaborolan-2-yl)indole-2-carboxylic acid ethyl ester

The sub-title compound was prepared in accordance with Example 30, step (d) from 5-bromo-3-chloroindole-2-carboxylic acid ethyl ester (see Example 34, step (a)) and bis(pinacolato)diboron.

### (b) 3-Chloro-5-(5-trifluoromethylpyridin-2-yl)indole-2-carboxylic acid ethyl ester

The sub-title compound was prepared in accordance with Example 30, step (e) from 3-chloro-5-(4,4,5,5-tetramethyl-[1,3,2]dioxaborolan-2-yl)indole-2-carboxylic acid ethyl ester (see step (a) above) and 2-bromo-5-(trifluoromethyl)pyridine.

### (c) 3-Chloro-1-(4-isopropoxyphenyl)-5-(5-trifluoromethylpyridin-2-yl)indole-2-carboxylic acid ethyl ester

The sub-title compound was prepared in accordance with Example 30, step (c) with 3-chloro-5-(5-trifluoromethylpyridin-2-yl)indole-2-carboxylic acid ethyl ester (see step (b) above) and 4-isopropoxyboronic acid.

### (d) [3-Chloro-1-(4-isopropoxyphenyl)-5-(5-trifluoromethylpyridin-2-yl)indol-2-yl]methanol

The sub-title compound was prepared in accordance with Example 29, step (c) from 3-chloro-1-(4-isopropoxyphenyl)-5-(5-trifluoromethylpyridin-2-yl)indole-2-carboxylic acid ethyl ester (see step (c) above).

### (e) 3-Chloro-1-(4-isopropoxy-phenyl)-5-(5-trifluoromethyl-pyridin-2-yl)indole-2-carbaldehyde

The sub-title compound was prepared in accordance with Example 29, step (d) from [3-chloro-1-(4-isopropoxyphenyl)-5-(5-trifluoromethylpyridin-2-yl)indol-2-yl]methanol (see step (d) above).

### (f) 3-[3-Chloro-1-(4-isopropoxyphenyl)-5-(5-trifluoromethylpyridin-2-yl)indol-2-yl]acryl ic acid ethyl ester

The sub-title compound was prepared in accordance with Example 29, step (e) from 3-chloro-1-(4-isopropoxyphenyl)-5-(5-trifluoromethylpyridin-2-yl)indole-2-carbaldehyde (see step (e) above) and triphenylphosphanylidene acetic acid ethyl ester.

### (g) 3-[3-Chloro-1-(4-isopropoxyphenyl)-5-(5-trifluoromethylpyridin-2-yl)indol-2-yl]acrylic acid

The title compound was prepared in accordance with Example 29, step (g) from 3-[3-chloro-1-(4-isopropoxyphenyl)-5-(5-trifluoromethylpyridin-2-yl)indol-2-yl]-acrylic acid ethyl ester (see step (f) above).

200 MHz ¹H-NMR (DMSO-d₆, ppm) δ12.7-12.5 (1H, br s) 9.05-9.01 (1H, m) 8.49-8.45 (1H, m) 8.34-8.20 (2H, m) 8.14 (1H, dd, J=8.8, 1.6 Hz) 7.45-7.37 (2H, m) 7.36 (1H, d, J=16 Hz) 7.21-7.12 (3H, m) 6.29 (1H, d, J=16 Hz) 4.74 (1H, septet, J=6.0 Hz) 1.33 (6H, d, J=6.0 Hz)

### Example 46

### 3-[1-(4-Isopropoxyphenyl)-5-(5-trifluoromethylpyridin-2-yl)indol-2-yl]propionic acid

### (a) 3-[1-(4-Isopropoxyphenyl)-5-(5-trifluoromethylpyridin-2-yl)indol-2-yl]-propionic acid ethyl ester

Cyclohexene (2.0 mL) and 10% Pd—C (120 mg, 1.13 mmol) were added to a solution of 3-[3-chloro-1-(4-isopropoxyphenyl)-5-(5-trifluoromethylpyridin-2-yl)-indol-2-yl]acrylic acid ethyl ester (see Example 45 step (f)) in EtOH (3 mL). The mixture was heated at 135° C. for 20 min by microwave irradiation and filtered through Celite®. The filtrate was concentrated to afford 190 mg (91%) of the sub-title product.

### (b) 3-[1-(4-Isopropoxyphenyl)-5-(5-trifluoromethylpyridin-2-yl)indol-2-yl]propionic acid

The title compound was prepared in accordance with Example 29, step (g) from 3-[1-(4-isopropoxyphenyl)-5-(5-trifluoromethylpyridin-2-yl)indol-2-yl]propionic acid ethyl ester (see step (a) above).

200 MHz ¹H-NMR (DMSO-d₆, ppm) δ 12.22 (1H, s) 8.98-8.94 (1H, m) 8.39-8.35 (1H, m) 8.24-8.11 (2H, m) 7.89 (1H, dd, J=8.5, 1.6 Hz) 7.38-7.30 (2H, m) 7.15-7.08 (2H, m) 7.04 (1H, d, J=8.8 Hz) 6.52 (1H, s) 4.71 (1H, septet, J=6.0 Hz) 2.84-2.73 (2H, m) 2.63-2.53 (2H, m) 1.32 (6H, d, J=6.0 Hz)

### Example 47

### [5-(4-Chloro-3-trifluoromethoxyphenyl)-1-(4-isopropoxyphenyl)-3-methylindol-2-yl]phosphonic acid monoethyl ester sodium salt

### (a) Propionylphosphonic Acid Diethyl Ester

Propionyl chloride (8.9 mL, 100 mmol) was added dropwise to phosphorous acid triethyl ester (16.7 mL, 100 mmol) at 0° C. After 1 h at 0° C., the mixture was stirred overnight at rt. Concentration and distillation (bp 200-210° C. at 11 Torr) afforded 12.8 g (66%) of the sub-title compound.

### (b) (5-Bromo-3-methylindol)-2-phosphonic acid diethyl ester

To a suspension of N′-(4-bromophenyl)hydrazinium chloride (7.83 g, 35 mmol) in anhydrous toluene (70 mL) was added propionyl phosphonic acid diethyl ester (6.79 g, 35 mmol; see step (a) above). After stirring for 5 min under argon, polyphosphoric acid (14 g) was added and the reaction was heated at reflux for 5 min. The clear solution was poured into water (200 mL), extracted with t-BuOMe (3×100 mL) and the combined extracts were washed with brine and dried (Na₂SO₄). Concentration afforded an oily residue which was purified by chromatography to afford the sub-title compound (5.82 g, 48%).

### (c) [5-Bromo-1-(4-isopropoxyphenyl)-3-methylindol]-2-phosphonic acid diethyl ester

The sub-title compound was prepared in accordance with Example 29, step (b), Method B from (5-bromo-3-methylindol)-2-phosphonic acid diethyl ester (see step (b) above) and 4-isopropoxyphenylboronic acid.

### (d) 4-Bromo-1-chloro-2-trifluoromethoxybenzene

NaNO₂ (2.43 g, 0.035 mol) in water (10 mL) was added in portions over 30 min to 4-bromo-2-trifluoromethoxyaniline (9 g, 35 mmol) in a mixture of HCl (aq, conc, 25 mL) and water (25 mL) at (0-2° C.). The mixture was stirred at 0-2° C. for 15 min and CuCl (6 g, 61 mmol) in HCl (aq, cone, 10 mL) was added dropwise. After 10 min at rt, the mixture was heated at reflux for 15 min. Steam-distillation followed by extraction (CH₂Cl₂), drying (Na₂SO₄) of the distillate followed by concentration and distillation (bp 82-84° C. at 20 Torr) gave 3.86 g (40%) of the sub-title compound.

### (e) 4-chloro-3-trifluoromethoxyphenyl boronic acid

n-BuLi (2.5 M in hexanes; 6.25 mL, 12.5 mmol) was added dropwise to 4-bromo-1-chloro-2-trifluoromethoxybenzene (3.4 g, 12.3 mmol; see step (d) above) in anhydrous THF (50 mL) at −78° C. After 30 min, triethylborate (2.1 mL, 12.5 mmol) was added and the mixture was allowed to warm to rt and stirred at rt for 2 h. The mixture was poured into water (100 mL), acidified to pH 4 with HCl (aq, 1 M) and extracted with EtOAc (3×50 mL). The combined extracts were washed with brine, dried (Na₂SO₄) and concentrated. The residue was recrystallised from petroleum ether to yield 2.07 g (70%) of the sub-title compound.

### (f) [5-(4-Chloro-3-trifluoromethoxyphenyl)-1-(4-isopropoxyphenyl)-3-methylindol-2-yl]-phosphonic acid diethyl ester

The sub-title compound was prepared in accordance with Example 29, step (a) from [5-bromo-1-(4-isopropoxy-phenyl)-3-methylindol-2-yl]phosphonic acid diethyl ester (see step (c) above) and 4-chloro-3-trifluoromethoxyphenyl boronic acid (see step (e) above).

### (g) [5-(4-Chloro-3-trifluoromethoxyphenyl)-1-(4-isopropoxyphenyl)-3-methylindol]-2-phosphonic acid monoethyl ester sodium salt

A mixture of [5-(4-chloro-3-trifluoromethoxyphenyl)-1-(4-isopropoxyphenyl)-3-methylindol]-2-phosphonic acid diethyl ester (290 mg, 0.49 mmol, see step (f) above), NaOH (aq, 2 M, 2 mL) and dioxane (3 mL) was heated by microwave irradiation at 140° C. for 2 h, cooled and acidified with HCl (aq, 1 M) to pH 2. The mixture was extracted with EtOAc (3×10 mL) and the combined extracts were washed with water and brine, dried (Na₂SO₄), concentrated and purified by reverse-phase HPLC affording 111 mg (40%) of the title compound.

200 MHz ¹H-NMR (DMSO-d₆, ppm) δ 7.90-7.29 (7H, m) 7.01-6.87 (3H, m) 4.74-4.37 (1H, m) 3.50-3.25 (2H, m, overlapped with water) 2.62 (3H, s) 1.32 (4H, d, J=6.0 Hz) 1.26-1.12 (2H, m) 0.82 (2H, t, J=6.5 Hz) 0.76-0.58 (1H, m).

### Example 48

### [1-(4-Isopropoxyphenyl)-5-(4-isopropoxy-3-trifluoromethoxyphenyl)-3-methylindol]-2-phosphonic acid monoethyl ester sodium salt

### (a) 4-Bromo-2-trifluoromethoxyphenol

Bromine (1.0 M in CH₂Cl₂, 45 mL, 45 mmol) was added dropwise over 20 min to a solution of 2-trifluoromethoxyphenol (7.40 g, 41.5 mmol) in CH₂Cl₂ (100 mL) at −78° C. The mixture was allowed to warm to rt and was stirred at rt for 48 h. Na₂SO₃ (aq, sat, 100 mL) was added and the mixture was stirred vigorously until the orange colour disappeared. The mixture was diluted with CH₂Cl₂ (200 mL) and the organic layer was washed with brine, dried (Na₂SO₄) and concentrated to afford 9.6 g (91%) of the sub-title product.

### (b) 4-Bromo-1-isopropoxy-2-trifluoromethoxybenzene

A mixture of 4-bromo-2-trifluoromethoxyphenol (9.6 g, 37.4 mmol), 2-bromopropane (7.0 mL, 74.7 mmol) and NaOH (3.0 g, 74.7 mmol) in anhydrous DMF (25 mL) was heated at 70° C. for 2 h, poured into water (100 mL) and extracted with t-BuOMe (3×100 mL). The combined extracts were washed with brine, dried (Na₂SO₄), concentrated and distilled (bulb-to bulb, 150° C., 9.8×10⁻² Torr) to yield 9.5 g (85%) of the sub-title compound.

### (c) 4-Isopropoxy-3-trifluoromethoxyphenyl boronic acid

The sub-title compound was prepared in accordance with Example 47, step (e) from 4-bromo-1-isopropoxy-2-trifluoromethoxybenzene (see step (b) above).

### (d) 1-(4-Isopropoxyphenyl)-5-(4-isopropoxy-3-trifluoromethoxyphenyl)-3-methylindol]-2-phosphonic acid diethyl ester

The sub-title compound was prepared in accordance with Example 29, step (a) from [5-bromo-1-(4-isopropoxyphenyl)-3-methylindol]-2-phosphonic acid diethyl ester (see step Example 47, step (c)) and 4-isopropoxy-3-trifluoromethoxyphenyl boronic acid (see step (e) above).

### (e) [1-(4-Isopropoxyphenyl)-5-(4-isopropoxy-3-trifluoromethoxyphenyl)-3-methylindol]-2-phosphonic acid monoethyl ester sodium salt

The title compound was prepared in accordance with Example 47, step (g) from 1-(4-isopropoxyphenyl)-5-(4-isopropoxy-3-trifluoromethoxyphenyl)-3-methylindol]-2-phosphonic acid diethyl ester (see step (d) above).

600 MHz ¹H-NMR (DMSO-d₆, ppm) δ 7.77-7.73 (1H, m) 7.65-7.61 (1H, m) 7.60-7.57 (1H, m) 7.48-7.31 (3H, m) 7.30 (1H, d, J=8.8 Hz) 6.98-6.88 (3H, m) 4.73 (1H, septet, J=6.0 Hz) 4.65 (0.7H, septet, J=6.0 Hz) 4.54-4.44 (0.3H, m) 3.51-3.35 (2H, m) 2.61 (3H, s) 1.32 (6H, d, J=6.0 Hz) 1.30-1.15 (6H, m) 0.82 (2.3H, t, J=6.6 Hz) 0.73-0.55 (0.7H, m)

### Example 49

### 3-Chloro-5-(4-chloro-3-trifluoromethoxyphenoxy)-1-(4-isopropoxyphenyl)-2-(tetrazol-5-yl)indole

### (a) 5-Benzyloxy-1-(4-isopropoxyphenyl)indole-2-carboxylic acid ethyl ester

An oven dried pressure tube (35 mL) was charged with K₃PO₄ (2.9 g, 13.7 mmol), 5-benzyloxyindole-2-carboxylic acid ethyl ester (2.0 g, 6.77 mmol) and flushed with argon. A solution of 4-isopropoxyphenylbromide (1.75 g, 8.14 mmol) in toluene (7.0 mL) was added, followed by a solution of CuI (193 mg, 1.01 mmol) and N,N′-dimethyl-1,2-diaminoethane (216 μL, 2.03 mmol) in toluene (5.0 mL). The mixture was heated at 90° C. for 48 h, cooled, poured into NH₄Cl (aq, sat, 50 mL) and extracted with EtOAc (3×50 mL). The combined extracts were washed with brine, dried (Na₂SO₄), filtered through silica gel and concentrated. The solid residue was recrystallised from EtOAc/petroleum ether to afford 2.5 g (86%) of the sub-title compound.

### (b) 5-Hydroxy-1-(4-isopropoxyphenyl)indole-2-carboxylic acid ethyl ester

A solution of 5-benzyloxy-1-(4-isopropoxyphenyl)indole-2-carboxylic acid ethyl ester (2.0 g, 4.6 mmol; see step (a) above) in EtOAc (30 mL) and EtOH (30 mL) was hydrogenated at ambient temperature and pressure over 10% Pd on carbon (490 mg, 0.546 mmol) for 2 h. The mixture was filtered through silica gel, concentrated and crystallised from EtOAc/petroleum ether to give the sub-title compound (1.3 g, 83%).

### (c) 5-Acetoxy-1-(4-isopropoxyphenyl)indole-2-carboxylic acid ethyl ester

Acetyl chloride (850 μL, 11.9 mmol) was added to a solution of 5-hydroxy-1-(4-isopropoxyphenyl)indole-2-carboxylic acid ethyl ester (2.7 g, 7.96 mmol; see step (b) above), DMAP (486 mg, 3.98 mmol) and Et₃N (3.4 mL, 23.9 mmol) in anhydrous CH₂Cl₂ (80 mL). After 12 h at rt, the mixture was poured into water (100 mL). HCl (1M, 100 mL) was added and the mixture was extracted with EtOAc (3×50 mL). The combined extracts were washed with brine, dried (Na₂SO₄) and concentrated to afford 2.9 g (95%) of the sub-title compound.

### (d) 5-Acetoxy-3-chloro-1-(4-isopropoxyphenyl)indole-2-carboxylic acid ethyl ester

SO₂Cl₂ (950 μL, 11.8 mmol) was added dropwise over 15 min to a solution of 5-acetoxy-1-(4-isopropoxyphenyl)indole-2-carboxylic acid ethyl ester (4.5 g, 11.8 mmol; see step (c) above) in anhydrous CH₂Cl₂ (200 mL) at 0° C. (dry ice bath). After 2 h at 0° C., the mixture was poured into NaHCO₃ (aq, sat, 200 mL) and extracted with EtOAc (3×100 mL). The combined extracts were washed with water and brine, dried (Na₂SO₄) and concentrated to afford 4.0 g (82%) of the sub-title compound.

### (e) 3-Chloro-5-hydroxy-1-(4-isopropoxyphenyl)indole-2-carboxylic acid ethyl ester

5-Acetoxy-3-chloro-1-(4-isopropoxyphenyl)indole-2-carboxylic acid ethyl ester (1.41 g, 3.39 mmol; see step (d) above) was dissolved in MeOH saturated with ammonia (75 mL). The solution was kept at 5° C. for 20 h and concentrated. The residue was dissolved in CH₂Cl₂, filtered through silica gel and concentrated to afford 1.16 g (91%) of the sub-title compound.

### (f) 3-Chloro-1-(4-isopropoxyphenyl)-5-(4-chloro-3-trifluoromethoxyphenoxy)-indole-2-carboxylic acid ethyl ester

Anhydrous CH₂Cl₂ (60 mL), Et₃N (380 μL, 2.68 mmol) and pyridine (220 mL, 2.68 mmol) were added to 3-chloro-5-hydroxy-1-(4-isopropoxyphenyl)indole-2-carboxylic acid ethyl ester (500 mg, 1.34 mmol; see step (e) above), Cu(OAc)₂ (487 mg, 2.68 mmol) and 4-chloro-3-trifluoromethoxyphenyl boronic acid (644 mg, 2.68 mmol; see Example 47, step (e)). The mixture was vigorously stirred at rt for 24 h. After the reaction was complete (as judged by TLC), the mixture was filtered through Celite®, concentrated and purified by chromatography to afford the sub-title compound (492 mg, 65%).

### (g) 3-Chloro-5-(4-chloro-3-trifluoromethoxyphenoxy)-1-(4-isopropoxyphenyl)-indole-2-carboxylic acid

The sub-title compound was prepared in accordance with Example 29, step (g) from 3-chloro-1-(4-isopropoxyphenyl)-5-(4-trifluoromethoxyphenoxy)indole-2-carboxylic acid ethyl ester (see step (f) above).

### (h) 3-Chloro-5-(4-chloro-3-trifluoromethoxyphenoxy)-1-(4-isopropoxyphenyl)-indole-2-carbonitrile

The sub-title compound was prepared in accordance with Example 30, steps (a) and (b) from 3-chloro-1-(4-isopropoxyphenyl)-5-(4-trifluoromethoxyphenoxy)-indole-2-carboxylic acid (see step (g) above).

### (i) 3-Chloro-5-(4-chloro-3-trifluoromethoxyphenoxy)-1-(4-isopropoxyphenyl)-2-(tetrazol-5-yl)indole

The title compound was prepared in accordance with Example 30, step (f) from 3-chloro-5-(4-chloro-3-trifluoromethoxyphenoxy)-1-(4-isopropoxyphenyl)indole-2-carbonitrile (see step (h) above).

200 MHz ¹H-NMR (DMSO-d₆, ppm) δ 7.66 (1H, d, J=9.0 Hz) 7.74 (1H, d, J=2.2 Hz)) 7.35-7.19 (4H, m) 7.16 (1H, d, J=9.0; 2.2 Hz) 7.08-6.93 (3H, m) 4.65 (1H, septet, J=6.0 Hz) 1.29 (6H, d, J=6.0 Hz).

### Example 50

### 3-Chloro-1-(4-isopropoxyphenyl)-2-(tetrazol-5-yl)-5-(4-trifluoromethoxyphenoxy)indole

The title compound was prepared in accordance with Example 49 from 3-chloro-5-hydroxy-1-(4-isopropoxyphenyl)indole-2-carboxylic acid ethyl ester (Example 49, step (e)) and 4-trifluoromethoxybenzene boronic acid, followed by the conversion to the tetrazole (see Example 49, steps (g-i)).

200 MHz ¹H-NMR (DMSO-d₆, ppm) δ 7.44-7.32 (3H, m) 7.32-7.20 (3H, m) 7.19-7.06 (3H, m) 7.04-6.94 (2H, m) 4.65 (1H, septet, J=6.0 Hz) 1.29 (6H, d, J=6.0 Hz).

### Example 51

The following compounds are prepared in accordance with techniques described herein:
1-(4-isopropoxyphenyl)-3-methyl-5-(5-trifluormethylpyridin-2-yl)indole-2-carboxylic acid;
1-(4-cyclopentyloxyphenyl)-5-(6-methyl-5,6,7,8-tetrahydroquinolin-2-yl)-3-trifluoromethyl indole-2-carboxylic acid;
3-cyclohexyl-1-(4-cyclopentyloxyphenyl)-5-(5-trifluormethylpyridin-2-yl)indole-2-carboxylic acid;
1-(4-cyclopentyloxyphenyl)-3-(piperidin-3-yl)-5-(4-trifluoromethylphenyl)indole-2-carboxylic acid; and
1-(4-isopropoxyphenyl)-3-(trifluoromethyl)-5-(5-(trifluoromethyl)pyridin-2-yl)-indole-2-carboxylic acid;
5-(4-cyclohexylphenyl)-1-(4-isopropoxyphenyl)indol-2-boronic acid;
5-(4-cyclohexylphenyl)-1-(4-isopropoxyphenyl)indole-2-sulfonic acid;
5-(4-cyclohexylphenyl)-1-(4-isopropoxyphenyl)indol-2-phosphonic acid;
3-(1-(4-isopropoxyphenyl)-5-(6-isopropoxypyridin-3-yl)-3-(trifluoromethyl)indol-2-yl)-2,2-dimethyl-3-oxopropanoic acid; and
4-(1-(4-isopropoxyphenyl)-5-(6-isopropoxypyridin-3-yl)-3-(trifluoromethyl)indol-2-yl)-4-oxobutanoic acid.

### Example 52

Title compounds of the examples were tested in the biological test described above and were found to exhibit 50% inhibition of mPGES-1 at a concentration of 10 μM or below. For example, the following representative compounds of the examples exhibited the following IC₅₀ values:

### Example 2: 2600 nM

### Example 8: 560 nM

### Example 9: 2100 nM

### Example 29: 780 nM

Example 32: 3200 nM

1. A compound of formula I, 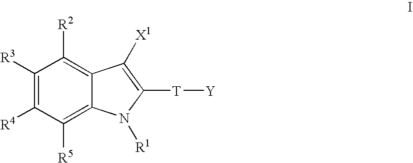
wherein
one of the groups R², R³, R⁴ and R⁵ represents -D-E and:
a) the other groups are independently selected from hydrogen, G¹, an aryl group, a heteroaryl group (which latter two groups are optionally substituted by one or more substituents selected from A), C₁₋₈ alkyl and a heterocycloalkyl group (which latter two groups are optionally substituted by one or more substituents selected from G¹ and/or Z¹); and/or
b) any two other groups which are adjacent to each other are optionally linked to form, along with two atoms of the essential benzene ring in the compound of formula I, a 3- to 8-membered ring, optionally containing 1 to 3 heteroatoms, which ring is itself optionally substituted by one or more substituents selected from halo, —R⁶, —OR⁶ and ═O;
D represents a single bond, —O—, —C(R⁷)(R⁸)—, C₂₋₄ alkylene, —C(O)— or —S(O)ₘ—;
R¹ and E independently represent an aryl group or a heteroaryl group, both of which groups are optionally substituted by one or more substituents selected from A;
R⁷ and R⁸ independently represent H, halo or C₁₋₆ alkyl, which latter group is optionally substituted by halo, or R⁷ and R⁸ may together form, along with the carbon atom to which they are attached, a 3- to 6-membered ring, which ring optionally contains a heteroatom and is optionally substituted by one or more substituents selected from halo and C₁₋₃ alkyl, which latter group is optionally substituted by one or more halo substituents;
X¹ represents H, halo, —N(R^{9a})-J-R^{10a} or -Q-X²;
J represents a single bond, —C(O)— or —S(O)ₘ—;
Q represents a single bond, —O—, —C(O)— or —S(O)ₘ—;
X² represents:
(a) an aryl group or a heteroaryl group, both of which are optionally substituted by one or more substituents selected from A; or
(b) C₁₋₈ alkyl or a heterocycloalkyl group, both of which are optionally substituted by one or more substituents selected from G¹ and/or Z¹; or, when Q is a single bond,
(c) cyano;
T represents:
(a) a single bond;
(b) a C₁₋₈ alkylene or a C₂₋₈ heteroalkylene chain, both of which latter two groups:
(i) optionally contain one or more unsaturations;
(ii) are optionally substituted by one or more substituents selected from G¹ and/or Z¹; and/or
(iii) may comprise an additional 3- to 8-membered ring formed between any one or more members of the C₁₋₈ alkylene or C₂₋₈ heteroalkylene chain, which ring optionally contains 1 to 3 heteroatoms and/or 1 to 3 unsaturations and which ring is itself optionally substituted by one or more substituents selected from G¹ and/or Z¹;
(c) an arylene group or a heteroarylene group, both of which groups are optionally substituted by one or more substituents selected from A; or
(d) -T¹-W¹-T²-;
one of T¹ and T² represents a C₁₋₈ alkylene or a C₂₋₈ heteroalkylene chain, both of which latter two groups:
(i) optionally contain one or more unsaturations;
(ii) are optionally substituted by one or more substituents selected from G¹ and/or Z¹; and/or
(iii) may comprise an additional 3- to 8-membered ring formed between any one or more members of the C₁₋₈ alkylene or C₂₋₈ heteroalkylene chain, which ring optionally contains 1 to 3 heteroatoms and/or 1 to 3 unsaturations and which ring is itself optionally substituted by one or more substituents selected from G¹ and/or Z¹;
and the other represents an arylene group or a heteroarylene group chain, both of which groups are optionally substituted by one or more substituents selected from A;
W¹ represents —O— or —S(O)ₘ—;
m represents 0, 1 or 2;
Y represents —C(H)(CF₃)OH, —C(O)CF₃, —C(OH)₂CF₃, —C(O)OR^{9b}, —S(O)₃R^{9c}, —P(O)(OR^{9d})₂, —P(O)(OR^{9c})N(R^{10f})R^{9f}, —P(O)(N(R^{10g})R^{9g})₂, —B(OR^{9h})₂, —C(CF₃)₂OH, —S(O)₂N(R¹⁰ⁱ)R⁹ⁱ or any one of the following groups: 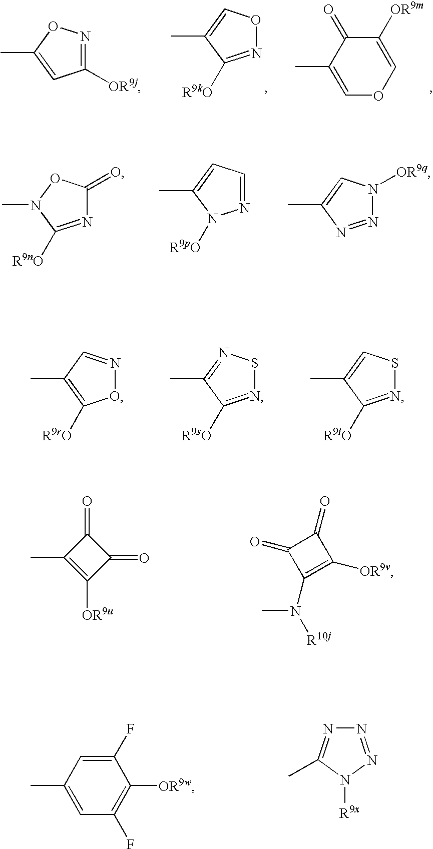
R⁶, R^{9a} to R^{x}, R^{10a}, R^{10f}, R^{10g}, R¹⁰ⁱ and R^{10j} independently represent:
I) hydrogen;
II) an aryl group or a heteroaryl group, both of which are optionally substituted by one or more substituents selected from B; or
III) C₁₋₈ alkyl or a heterocycloalkyl group, both of which are optionally substituted by one or more substituents selected from G¹ and/or Z¹; or
any pair of R^{9a} to R^{9x} and R^{10a}, R^{10f}, R^{10g}, R¹⁰ⁱ or R^{10j}, may be linked together to form, along with the atom(s) and/or group(s) to which they are attached, a 3- to 8-membered ring, optionally containing 1 to 3 heteroatoms and/or 1 to 3 double bonds, which ring is optionally substituted by one or more substituents selected from G¹ and/or Z¹;
A represents:
I) an aryl group or a heteroaryl group, both of which are optionally substituted by one or more substituents selected from B;
II) C₁₋₈ alkyl or a heterocycloalkyl group, both of which are optionally substituted by one or more substituents selected from G¹ and/or Z¹; or
III) a G¹ group;
G¹ represents halo, cyano, —N₃, —NO₂, —ONO₂ or -A¹-R^{11a};
wherein A¹ represents a single bond or a spacer group selected from —C(O)A²-, —S(O)₂A³-, —N(R^{12a})A⁴- or —OA⁵-, in which:
A² represents a single bond, —O—, —N(R^{12b})— or —C(O)—;
A³ represents a single bond, —O— or —N(R^{12c})—;
A⁴ and A⁵ independently represent a single bond, —C(O)—, —C(O)N(R^{12d})—, —C(O)O—, —S(O)₂— or —S(O)₂N(R^{12e})—;
Z¹ represents ═O, ═S, ═NOR^{11b}, ═NS(O)₂N(R^{12f})R^{11c}, ═NCN or ═C(H)NO₂;
B represents:
I) an aryl group or a heteroaryl group, both of which are optionally substituted by one or more substituents selected from G²;
II) C₁₋₈ alkyl or a heterocycloalkyl group, both of which are optionally substituted by one or more substituents selected from G² and/or Z²; or
III) a G² group;
G² represents halo, cyano, —N₃, —NO₂, —ONO₂ or -A⁶-R^{13a};
wherein A⁶ represents a single bond or a spacer group selected from —C(O)A⁷-, —S(O)₂A⁸-, —N(R^{14a})A⁹- or —OA¹⁰-, in which:
A⁷ represents a single bond, —O—, —N(R^{14b})— or —C(O)—;
A⁸ represents a single bond, —O— or —N(R^{14c})—;
A⁹ and A¹⁰ independently represent a single bond, —C(O)—, —C(O)N(R^{14d})—, —C(O)O—, —S(O)₂— or —S(O)₂N(R^{14e})—;
Z² represents ═O, ═S, ═NOR^{13b}, ═NS(O)₂N(R^{14f})R^{13c}, ═NCN or ═C(H)NO₂;
R^{11a}, R^{11b}, R^{11c}, R^{12a}, R^{12b}, R^{12c}, R^{12d}, R^{12e}, R^{12f}, R^{13a}, R^{13b}, R^{13c}, R^{14a}, R^{14b}, R^{14c}, R^{14d}, R^{14e} and R^{14f} are independently selected from:
i) hydrogen;
ii) an aryl group or a heteroaryl group, both of which are optionally substituted by one or more substituents selected from G³;
iii) C₁₋₈ alkyl or a heterocycloalkyl group, both of which are optionally substituted by G³ and/or Z³; or
any pair of R^{11a} to R^{11c} and R^{12a} to R^{12f}, and/or R^{13a} to R^{13c} and R^{14a} to R^{14f}, may be linked together to form with those, or other relevant, atoms a further 3- to 8-membered ring, optionally containing 1 to 3 heteroatoms and/or 1 to 3 double bonds, which ring is optionally substituted by one or more substituents selected from G³ and/or Z³;
G³ represents halo, cyano, —N₃, —NO₂, —ONO₂ or -A¹¹-R^{15a};
wherein A¹¹ represents a single bond or a spacer group selected from —C(O)A^{l2}-, —S(O)₂A¹³-, —N(R^{16a})A¹⁴- or —OA¹⁵-, in which:
A^{l2} represents a single bond, —O—, —N(R^{16b})— or —C(O)—;
A¹³ represents a single bond, —O— or —N(R^{16c})—;
A¹⁴ and A¹⁵ independently represent a single bond, —C(O)—, —C(O)N(R^{16d})—, —C(O)O—, —S(O)₂— or —S(O)₂N(R^{16e})—;
Z³ represents ═O, ═S, ═NOR^{15b}, ═NS(O)₂N(R^{16f})R^{15c}, ═NCN or ═C(H)NO₂;
R^{15a}, R^{15b}, R^{15c}, R^{16a}, R^{16b}, R^{16c}, R^{16d}, R^{16e} and R^{16f} are independently selected from:
i) hydrogen;
ii) C₁₋₆ alkyl or a heterocycloalkyl group, both of which groups are optionally substituted by one or more substituents selected from halo, C₁₋₄ alkyl, —N(R^{17a})R^{18d}, —OR^{17b} and ═O; and
iii) an aryl or heteroaryl group, both of which are optionally substituted by one or more substituents selected from halo, C₁₋₄ alkyl, —N(R^{17c})R^{18b} and —OR^{17d}; or
any pair of R^{15a} to R^{15c} and R^{16a} to R^{16f} may be linked together to form with those, or other relevant, atoms a further 3- to 8-membered ring, optionally containing 1 to 3 heteroatoms and/or 1 to 3 double bonds, which ring is optionally substituted by one or more substituents selected from halo, C₁₋₄ alkyl, —N(R^{17e})R^{18c}, —OR^{17f} and ═O;
R^{17a}, R^{17b}, R^{17c}, R^{17d}, R^{17e}, R^{17f}, R^{18a}, R^{18b} and R^{18c} are independently selected from hydrogen and C₁₋₄ alkyl, which latter group is optionally substituted by one or more halo groups;
wherein:
(I) when X¹ represents H, halo, —N(R^{9a})-J-R^{10a} or -Q-X² in which Q is a single bond and X² is an aryl or heteroaryl group (both of which are optionally substituted by one or more substituents selected from A), then T does not represent a single bond when Y is —C(O)OR^{9b}; and
(II) when T represents a single bond and Y represents —C(O)OR^{9b}, then D represents a single bond,
or a pharmaceutically-acceptable salt thereof,
provided that, when X¹ represents -Q-X², R², R⁴ and R⁵ all represent H, R³ represents -D-E, E represents unsubstituted phenyl, T represents a single bond, Y represents —C(O)OR^{9b}, R^{9b} represents ethyl, and R¹ represents 2,4-dinitrophenyl, then:
(a) when Q represents a single bond, X² does not represent methyl; and
(b) when Q represents —O—, X² does not represent methyl or ethyl.

2. A compound as claimed in claim 1, wherein A represents G¹ or C₁₋₆ alkyl optionally substituted by one or more G¹ groups.

3. A compound as claimed in claim 1 or claim 2, wherein G¹ represents halo, cyano or -A¹-R^{11a}.

4. A compound as claimed in claim 1, wherein, A¹ represents a single bond, —N(R^{12a})A⁴- or —OA⁵-.

5. A compound as claimed in claim 1, wherein A⁴ and A⁵ independently represent a single bond.

6. A compound as claimed in claim 1, wherein Z¹ represents ═O.

7. A compound as claimed in claim 1, wherein R² and/or R⁵ represent H.

8. A compound as claimed in claim 1, wherein T represents C₂₋₄ heteroalkylene, or, a single bond or linear or branched C₁₋₅ alkylene, which latter group is optionally substituted by one or more Z¹ substituent.

9. A compound as claimed in claim 1, wherein Y represents —C(O)OR^{9b}, —B(OR^{9h})₂, —S(O)₃R^{9c}, —P(O)(OR^{9d})₂, —S(O)₂N(R¹⁰ⁱ)R⁹ⁱ or a tetrazolyl group.

10. A compound as claimed in claim 1, wherein D represents a single bond or —O—.

11. A compound as claimed in claim 1, wherein X¹ represents halo, -Q-X² or H.

12. A compound as claimed in claim 1, wherein one of R⁴ and R³ represents -D-E and the other represents H.

13. A compound as claimed in claim 1, wherein X² represents cyano, or a 5- or 6-membered nitrogen-containing heterocycloalkyl group, or optionally unsaturated linear, branched or cyclic C₁₋₆ alkyl, which latter two groups are optionally substituted with one or more G¹ and/or Z¹ substituents.

14. A compound as claimed in claim 1, wherein Q represents —O—, —S— or a single bond.

15. A compound as claimed in claim 1, wherein R^{11a}, R^{11b} and R^{11c} independently represent H or, a heteroaryl group or an optionally branched, optionally unsaturated and/or optionally cyclic C₁₋₆ alkyl group, both of which groups are optionally substituted by one or more G³ groups.

16. A compound as claimed in claim 1, wherein R^{12a}, R^{12b}, R^{12c}, R^{12d}, R^{12e} and R^{12f} independently represent H or C₁₋₂ alkyl.

17. A compound as claimed in claim 1, wherein R¹, E and X² (when X² represents such aryl or heteroaryl groups) represent optionally substituted phenyl, naphthyl, pyrrolyl, furanyl, thienyl, pyrazolyl, imidazolyl, oxazolyl, isoxazolyl, thiazolyl, pyridyl, indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, and/or benzodioxanyl, groups.

18. A compound as claimed in claim 17, wherein R¹ and E independently represent optionally substituted phenyl, pyridyl or imidazolyl.

19. A compound as claimed in claim 17 or claim 18, wherein the optional substituents are selected from halo, cyano, —NO₂, C₁₋₆ alkyl (which alkyl group may be linear or branched, cyclic, part-cyclic, unsaturated and/or optionally substituted with one or more halo group), heterocycloalkyl (which heterocycloalkyl group is optionally substituted by one or more substituents selected from C₁₋₃ alkyl and ═O), —OR¹⁹, —N(R¹⁹)R²⁰, wherein R¹⁹ and R²⁰ independently represent H or C₁₋₆ alkyl (which alkyl group is optionally substituted by one or more halo groups).

20. A compound as claimed in claim 1, wherein G³ represents halo or -A¹¹-R^{15a} (in which A¹¹ represents a single bond, —N(R^{16a})— or —O—, R^{15a} represents H, C₁₋₂ alkyl or a nitrogen-containing heteroaryl group and R^{16a} represents C₁₋₂ alkyl).

21. A compound as claimed in claim 1, wherein R^{9a} to R^{9x} independently represent H or C₁₋₄ alkyl.

22. A compound as claimed in claim 1, wherein R^{10a}, R^{10f}, R^{10g}, R¹⁰ⁱ and R^{10j} independently represent C₁₋₃ alkyl or H.

23. A compound as defined in claim 1, but without the proviso, or a pharmaceutically-acceptable salt thereof, for use as a pharmaceutical.

24. A pharmaceutical formulation including a compound as defined in claim 1, but without the proviso, or a pharmaceutically-acceptable salt thereof, in admixture with a pharmaceutically acceptable adjuvant, diluent or carrier.

25. (canceled)

26. (canceled)

27. (canceled)

28. A method as claimed in claim 30, wherein the disease is inflammation.

29. A use method as claimed in claim 30 wherein the disease is asthma, chronic obstructive pulmonary disease, pulmonary fibrosis, inflammatory bowel disease, irritable bowel syndrome, inflammatory pain, fever, migraine, headache, low back pain, fibromyalgia, a myofascial disorder, a viral infection, a bacterial infection, a fungal infection, dysmenorrhea, a burn, a surgical or dental procedure, a malignancy, hyperprostaglandin E syndrome, classic Bartter syndrome, atherosclerosis, gout, arthritis, osteoarthritis, juvenile arthritis, rheumatoid arthritis, rheumatic fever, ankylosing spondylitis, Hodgkin's disease, systemic lupus erythematosus, vasculitis, pancreatitis, nephritis, bursitis, conjunctivitis, iritis, scleritis, uveitis, wound healing, dermatitis, eczema, psoriasis, stroke, diabetes mellitus, a neurodegenerative disorder, an autoimmune disease, an allergic disorder, rhinitis, an ulcer, coronary heart disease, sarcoidosis, any other disease with an inflammatory component, osteoporosis, osteoarthritis, Paget's disease or a periodontal disease.

30. A method of treatment of a disease in which inhibition of the activity of a member of the MAPEG family is desired and/or required, which method comprises administration of a therapeutically effective amount of a compound as defined in claim 1, but without the provisos, or a pharmaceutically-acceptable salt thereof, to a patient suffering from, or susceptible to, such a condition.

31. A method as claimed in claim 30, wherein the member of the MAPEG family is microsomal prostaglandin E synthase-1, leukotriene C₄ and/or 5-lipoxygenase-activating protein.

32. A method as claimed in claim 31, wherein the member of the MAPEG family is microsomal prostaglandin E synthase-1.

33. A combination product comprising:
(A) a compound as defined in claim 1, but without the provisos, or a pharmaceutically-acceptable salt thereof; and
(B) another therapeutic agent that is useful in the treatment of inflammation,
wherein each of components (A) and (B) is formulated in admixture with a pharmaceutically-acceptable adjuvant, diluent or carrier.

34. A combination product as claimed in claim 33 which comprises a pharmaceutical formulation including a compound as defined in any one of claims 1 to 22, but without the provisos, or a pharmaceutically-acceptable salt thereof, another therapeutic agent that is useful in the treatment of inflammation, and a pharmaceutically-acceptable adjuvant, diluent or carrier.

35. A combination product as claimed in claim 33 which comprises a kit of parts comprising components:
(a) a pharmaceutical formulation including a compound as defined in any one of claims 1 to 22, but without the provisos, or a pharmaceutically-acceptable salt thereof, in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
which components (a) and (b) are each provided in a form that is suitable for administration in conjunction with the other.

36. A process for the preparation of a compound as defined in claim 1, which comprises:
(i) reaction of a compound of formula II, 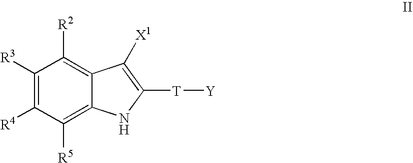
wherein X¹, R², R³, R⁴, R⁵, T and Y are as defined in claim 1, with a compound of formula III,
R¹L¹ III
wherein L¹ represents a suitable leaving group R¹ is as defined in claim 1;
(ii) for compounds of formula I in which X¹ represents -Q-X², in which Q is a single bond or —C(O)—, reaction of a compound of formula IV, 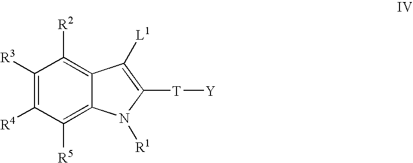
wherein R¹, R², R³, R⁴, R⁵, T and Y are as defined in claim 1 and L¹ is as defined above, with a compound of formula V,
X²-Q^{a}-L² V
wherein Q^{a} represents a single bond or —C(O)—, L² represents a suitable leaving group and X² is as defined in claim 1;
(iii) for compounds of formula I in which X¹ represents -Q-X² and Q represents —C(O)—, reaction of a compound of formula I in which X¹ represents H, with a compound of formula V in which Q^{a} represents —C(O)— and L² represents a suitable leaving group;
(iv) for compounds of formula I in which X¹ represents —N(R^{9a})-J-R^{10a} or -Q-X² in which Q represents —O— or —S—, reaction of a compound of formula IV as defined above with a compound of formula VI,
X^{1b}H VI
in which X^{1b} represents —N(R^{9a})-J-R^{10a} or -Q-X² in which Q represents —O— or —S— and R^{9a}, J, R^{10a} and X² are as defined in claim 1;
(v) for compounds of formula I in which X¹ represents -Q-X² and Q represents —S—, reaction of a compound of formula I in which X¹ represents H, with a compound of formula VI in which X^{1b} represents -Q-X², Q represents —S— and X² is as defined in claim 1;
(vi) for compounds of formula I in which X¹ represents -Q-X² and Q represents —S(O)— or —S(O)₂—, oxidation of a corresponding compound of formula I in which Q represents —S—;
(vii) for compounds of formula I in which X¹ represents -Q-X², X² represents C₁₋₈ alkyl substituted by G¹, G¹ represents -A¹-R^{11a}, A¹ represents —N(R^{12a})A⁴- and A⁴ is a single bond (provided that Q represents a single bond when X² represents substituted C₁ alkyl), reaction of a compound of formula VII, 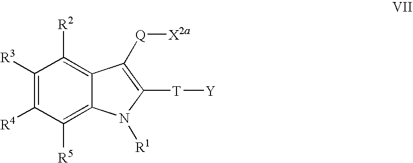
wherein X^{2a} represents a C₁₋₈ alkyl group substituted by a —Z¹ group in which Z¹ represents ═O, Q is as defined in claim 1, provided that it represents a single bond when X^{2a} represents C₁ alkyl substituted by ═O, and R¹, R², R³, R⁴, R⁵, T and Y are as defined in claim 1, under reductive amination conditions in the presence of a compound of formula VIII,
R^{11a}(R^{12a})NH VIII
wherein R^{11a} and R^{12a} are as defined in claim 1;
(viia) for compounds of formula I in which X¹ represents -Q-X², Q represents a single bond, X² represents methyl substituted by G¹, G¹ represents -A¹-R^{11a}, A¹ represents —N(R^{12a})A⁴- and A⁴ is a single bond, reaction of a corresponding compound of formula I in which X¹ represents H, with a mixture of formaldehyde (or equivalent reagent) and a compound of formula VIII as defined above;
(viii) for compounds of formula I in which X¹ represents -Q-X², Q represents a single bond and X² represents optionally substituted C₂₋₈ alkenyl (in which a point of unsaturation is between the carbon atoms that are É and é to the indole ring), reaction of a corresponding compound of formula IV in which L¹ represents halo with a compound of formula IXA,
H₂C═C(H)X^{2b} IXA
or reaction of a compound of formula VII in which Q represents a single bond and X^{2a} represents —CHO with either a compound of formula IXB,
(EtO)₂P(O)CH₂X^{2b} IXB
or the like, or a compound of formula IXC,
(Ph)₃P═CHX^{2b} IXC
or the like, wherein, in each case, X^{2b} represents H, G¹ or C₁₋₆ alkyl optionally substituted with one of more substituents selected from G¹ and/or Z¹ and G¹ and Z¹ are as defined in claim 1;
(ix) for compounds of formula I in which X¹ represents -Q-X² and X² represents optionally substituted, saturated C₂₋₈ alkyl, saturated cycloalkyl, saturated heterocycloalkyl, C₂₋₈ alkenyl, cycloalkenyl or heterocycloalkenyl, reduction of a corresponding compound of formula I in which X² represents optionally substituted C₂₋₈ alkenyl, cycloalkenyl, heterocycloalkenyl, C₂₋₈ alkynyl, cycloalkynyl or heterocycloalkynyl (as appropriate);
(x) for compounds of formula I in which D represents a single bond, —C(O)—, —C(R⁷)(R⁸)—, C₂₋₄ alkylene or —S(O)₂—, reaction of a compound of formula X, 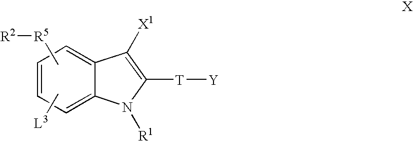
wherein L³ represents L¹ or L² as defined above, which group is attached to one or more of the carbon atoms of the benzenoid ring of the indole, R²-R⁵ represents whichever of the three other substituents on the benzenoid ring, i.e. R², R³, R⁴ and R⁵, are already present in that ring, and X¹, R¹, R², R³, R⁴, R⁵, T and Y are as defined in claim 1, with a compound of formula XI,
E-D^{a}-L⁴ XI
wherein D^{a} represents a single bond, —C(O)—, —C(R⁷)(R⁸)—, C₂₋₄ alkylene or —S(O)₂—, L⁴ represents L¹ (when L³ is L²) or L² (when L³ is L⁵, and E, R⁷ and R⁸ are as defined in claim 1 and L¹ and L² are as defined above;
(xi) for compounds of formula I in which D represents —S—, —O— or C₂₋₄ alkynylene in which the triple bond is adjacent to E, reaction of a compound of formula X as defined above in which L³ represents L² as defined above with a compound of formula XII,
E-D^{b}-H XII
wherein D^{b} represents —S—, —O— or C₂₋₄ alkynylene in which the triple bond is adjacent to E and E is as defined in claim 1;
(xii) for compounds of formula I in which D represents —S(O)— or —S(O)₂—, oxidation of a corresponding compound of formula I in which D represents —S—;
(xiii) for compounds of formula I in which D represents —O— or —S—, reaction of a compound of formula XIII, 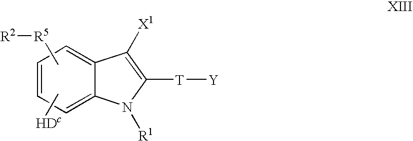
wherein the -D^{c}-H group is attached to one or more of the carbon atoms of the benzenoid ring of the indole, D^{c} represents —O— or —S—, and X¹, R¹, T and Y are as defined in claim 1, and R²-R⁵ is as defined above, with a compound of formula XIV,
E-L² XIV
wherein L² is as defined above and E is as defined in claim 1;
(xiv) for compounds of formula I in which X¹ represents —N(R^{9a})-J-R^{10a}, reaction of a compound of formula XV, 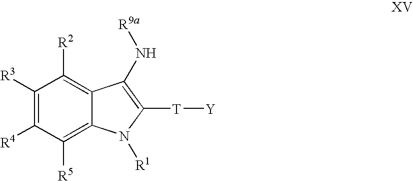
wherein R¹, R², R³, R⁴, R⁵, T, Y and R^{9a} are as defined in claim 1, with a compound of formula XVI,
R^{10a}-J-L¹ XVI
wherein J and R^{10a} are as defined in claim 1 and L¹ is as defined above;
(xv) for compounds of formula I in which X¹ represents —N(R^{9a})-J-R^{10a}, J represents a single bond and R^{10a} represents a C₁₋₈ alkyl group, reduction of a corresponding compound of formula I, in which J represents —C(O)— and R^{10a} represents H or a C₁₋₇ alkyl group;
(xvi) for compounds of formula I in which X¹ represents halo, reaction of a compound of formula I wherein X¹ represents H, with a reagent or mixture of reagents known to be a source of halide atoms;
(xvii) for compounds of formula I in which T and Y are as defined in claim 1, provided that when Y represents —C(O)OR^{9b}, —S(O)₃R^{9c}, —P(O)(OR^{9d})₂,
—P(O)(OR^{9e})N(R^{10f})R^{9f}, —P(O)(N(R^{10g})R^{9g})₂, —B(OR^{9h})₂ or —S(O)₂N(R¹⁰ⁱ)R⁹ⁱ, R^{9b} to R⁹ⁱ, R^{10f}, R^{10g} and R¹⁰ⁱ are other than H, reaction of a compound of formula XVII, 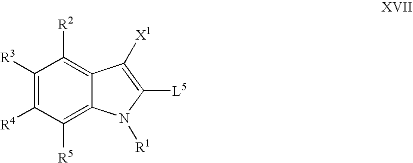
wherein L⁵ represents an appropriate alkali metal group, a —Mg-halide, a zinc-based group or a suitable leaving group, and X¹, R¹, R², R³, R⁴ and R⁵ are as defined in claim 1, with a compound of formula XVIII,
L⁶-T^{a}-Y^{a} XVIII
wherein T^{a} represents T and Y^{a} represents Y, provided that when Y represents —C(O)OR^{9b}, —S(O)₃R^{9c}, —P(O)(OR^{9d})₂, —P(O)(OR^{9e})N(R^{10f})R^{9f}, —P(O)(N(R^{10g})R^{9g})₂, —B(OR^{9h})₂ or —S(O)₂N(R¹⁰ⁱ)R⁹ⁱ, R^{9b} to R⁹ⁱ, R^{10f}, R^{10g} and R¹⁰ⁱ are other than H, and L⁶ represents a suitable leaving group;
(xviii) for compounds of formula I in which T represents a single bond, Y represents —B(OR^{9h})₂ and R^{9h} represents H, reaction of a compound of formula XVII as defined above with boronic acid or a protected derivative thereof, followed by (if necessary) deprotection;
(xix) for compounds of formula I in which T represents a single bond and Y represents —S(O)₃R^{9c}, reaction of a compound of formula XVII as defined above with:
(A) for such compounds in which R^{9c} represents H, either SO₃ or with SO₂ followed by treatment with N-chlorosuccinimide and then hydrolysis;
(B) for such compounds in which R^{9c} is other than H, chlorosulfonic acid followed by reaction with a compound of formula XXIII as defined below in which R^{9za} represents R^{9c};
(xx) for compounds of formula I in which T represents a single bond and Y represents 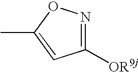
in which R^{9j} represents hydrogen, reaction of a corresponding compound of formula I in which T represents a C₂ alkylene group substituted at the carbon atom that is attached to the indole ring system by Z¹, in which Z¹ represents ═O and Y represents —C(O)OR^{9b}, in which R^{9b} represents C₁₋₆ alkyl with hydroxylamine or an acid addition salt thereof;
(xxi) for compounds of formula I in which T represents a single bond and Y represents 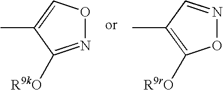
in which R^{9k} and R^{9r} represent hydrogen, reaction of a corresponding compound of formula I in which T represents a C₁ alkylene group substituted with G¹, in which G¹ represents -A¹-R^{11a}, A¹ represents —C(O)A²-, A² represents a single bond and R^{11a} represents H, and Y represents —C(O)OR^{9b}, in which R^{9b} represents methyl, or ethyl, respectively, with hydroxylamine or an acid addition salt thereof;
(xxii) for compounds of formula I in which T represents a single bond and Y represents 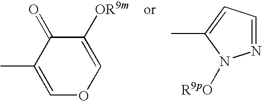
in which R^{9m} and R^{9p} represent hydrogen, reaction of a corresponding compound of formula I in which T represents a single bond, Y represents —B(OR^{9h})₂ and R^{9h} represents H with a compound of formula XVIII in which T^{a} represents a single bond, Y^{a} represents 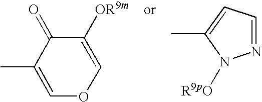
respectively, in which R^{9m} and R^{9p} represent hydrogen, and L⁶ represents a halo group, or a protected derivative of either compound;
(xxiii) for compounds of formula I in which T represents a single bond and Y represents 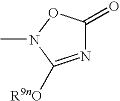
in which R⁹ⁿ represents hydrogen, reaction of a compound of formula XIX, 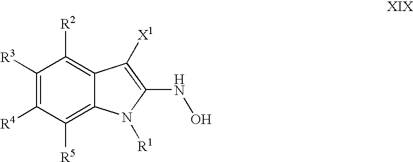
wherein X¹, R¹, R², R³, R⁴ and R⁵ are as defined in claim 1 with ethoxycarbonyl isocyanate;
(xxiv) for compounds of formula I in which T represents a single bond and Y represents 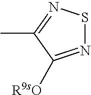
in which R^{9s} represents hydrogen, reaction of a compound of formula I in which T represents a single bond and Y represents —C(O)OR^{9b}, in which R^{9b} represents H with trimethylsilyl chloride, followed by reaction of the resultant intermediate with N₄S₄;
(xxv) for compounds of formula I in which T represents a single bond and Y represents 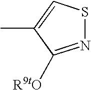
in which R^{9t} represents hydrogen, reaction of a compound of formula XX, 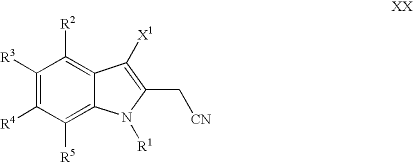
wherein X¹, R¹, R², R³, R⁴ and R⁵ are as defined in claim 1 with a base and CS₂, oxidation of the resultant intermediate, and heating the resultant intermediate in the presence of a strong acid;
(xxvi) for compounds of formula I in which T represents a single bond and Y represents 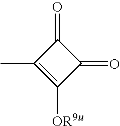
in which R^{9u} represents hydrogen, reaction of a corresponding compound of formula I in which T represents C₁ alkylene, Y represents —C(O)OR^{9b} and R^{9b} represents H or an activated derivative thereof with 1,1,2,2-tetraethoxyethene, followed by acid;
(xxvii) for compounds of formula I in which T represents a single bond and Y represents 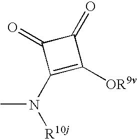
in which R^{9v} and R^{10j} independently represent H, reaction of a compound of formula XIX as defined above with 3,4-dimethoxycyclobutene-1,2-dione;
(xxviii) for compounds of formula I in which T represents a single bond and Y represents 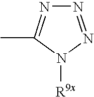
in which R^{9x} represents hydrogen, reaction of a compound of formula XXI, 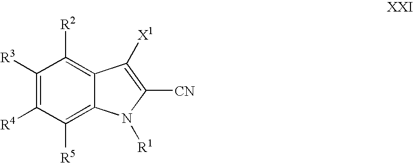
wherein X¹, R¹, R², R³, R⁴ and R⁵ are as defined in claim 1 with NaN₃;
(xxix) for compounds of formula I in which T represents optionally substituted C₂₋₈ alkenylene or C₂₋₈ heteroalkylene (in which a point of unsaturation is between the carbon atoms that are É and é to the indole ring), reaction of a compound of formula XXII, 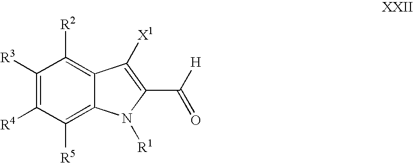
wherein X¹, R¹, R², R³, R⁴ and R⁵ are as defined in claim 1 with a compound of formula XXIIA,
(Ph)₃P═CH-T^{a}Y XXIIA
or the like, wherein T^{a} represents a single bond or optionally substituted C₁₋₆ alkylene or C₂₋₆ heteroalkylene and Y is as defined in claim 1;
(xxx) for compounds of formula I in which T represents optionally substituted, saturated C₂₋₈ alkylene, saturated cycloalkylene, saturated C₂₋₈ heteroalkylene, saturated heterocycloalkylene, C₂₋₈ alkenylene, cycloalkenylene, C₂₋₈ heteroalkenylene or heterocycloalkenylene, reduction of a corresponding compound of formula I in which T represents optionally substituted C₂₋₈ alkenylene, cycloalkenylene, C₂₋₈ heteroalkenylene, heterocycloalkenylene, C₂₋₈ alkynylene, cycloalkynylene, C₂₋₈ heteroalkynylene or heterocycloalkynylene (as appropriate);
(xxxi) for compounds of formula I in which Y represents —C(O)OR^{9b},
—S(O)₃R^{9c}, —P(O)(OR^{9d})₂, or —B(OR^{9h})₂, in which R^{9b}, R^{9c}, R^{9d} and R^{9h} represent H, hydrolysis of a corresponding compound of formula I in which R^{9b}, R^{9c}, R^{9d} or R^{9h} (as appropriate) does not represent H, or, for compounds of formula I in which Y represents —P(O)(OR^{9d})₂ or S(O)₃R^{9c}, in which R^{9c} and R^{9d} represent H, a corresponding compound of formula I in which Y represents either
—P(O)(OR^{9e})N(R^{10f})R^{9f}, —P(O)(N(R^{10f})R^{9g})₂ or —S(O)₂N(R¹⁰ⁱ)R⁹ⁱ (as appropriate);
(xxxii) for compounds of formula I in which Y represents —C(O)OR^{9b}, S(O)₃R^{9c}, —P(O)(OR^{9d})₂, —P(O)(OR^{9e})N(R^{10f})R^{9f} or —B(OR^{9h})₂ and R^{9b} to R^{9e} and R^{9h} do not represent H:
(A) esterification of a corresponding compound of formula I in which R^{9b} to R^{9e} and R^{9h} represent H; or
(B) trans-esterification of a corresponding compound of formula I in which R^{9b} to R^{9e} and R^{9h} do not represent H (and does not represent the same value of the corresponding R^{9b} to R^{9e} and R^{9h} group in the compound of formula I to be prepared),
in the presence of the appropriate alcohol of formula XXIII,
R^{9za}OH XXIII
in which R^{9za} represents R^{9b} to R^{9e} or R^{9h} provided that it does not represent H;
(xxxiii) for compounds of formula I in which T represents a single bond, Y represents —C(O)OR^{9b} and R^{9b} is other than H, reaction of a compound of formula XXIIIA, 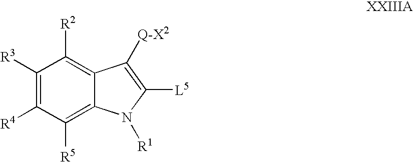
wherein Q, X², R¹, R², R³, R⁴ and R⁵ are as defined in claim 1 and L⁵ is as defined above, with a compound of formula XXIIIB,
L⁶C(O)OR^{9b1} XXIIIB
wherein R^{9b1} represents R^{9b} provided that it does not represent H, and L⁶ is as defined above;
(xxxiv) for compounds of formula I in which T represents a single bond, Y represents —C(O)OR^{9b} and R^{9b} is H, reaction of a compound of formula XXIIIA in which L⁵ represents either:
(I) an alkali metal; or
(II) —Mg-halide,
with carbon dioxide, followed by acidification;
(xxxv) for compounds of formula I in which T represents a single bond and Y represents —C(O)OR^{9b}, reaction of a corresponding compound of formula XXIIIA in which L⁵ is a suitable leaving group with CO (or a reagent that is a suitable source of CO), in the presence of a compound of formula XXIIIC,
R^{9b}OH XXIIIC
wherein R^{9b} is as defined in claim 1, and an appropriate catalyst system;
(xxxvi) for compounds of formula I in which Y represents —C(O)OR^{9b} and R^{9b} represents H, hydrolysis of a corresponding compound of formula I in which R^{9b} does not represent H;
(xxxvii) for compounds of formula I in which Y represents —C(O)OR^{9b} and R^{9b} does not represent H:
(A) esterification of a corresponding compound of formula I in which R^{9b} represents H; or
(B) trans-esterification of a corresponding compound of formula I in which R^{9b} does not represent H (and does not represent the same value of R^{9b} as the compound of formula Ito be prepared),
in the presence of the appropriate alcohol of formula XXIIIC as defined above but in which R^{9b} represents R^{9b1} as defined above;
(xxxviii) for compounds of formula I in which X¹ represents -Q-X² and Q represents —O—, reaction of a compound of formula XXIV, 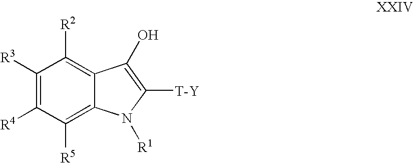
wherein R¹, R², R³, R⁴, R⁵, T and Y are as defined in claim 1, with a compound of formula XXV,
X²L⁷ XXV
wherein L⁷ represents a suitable leaving group, and X² is as defined in claim 1;
(xxxix) for compounds of formula I in which T represents a C₁ alkylene group substituted with G¹, in which G¹ represents -A¹-R^{11a}, A¹ represents
—C(O)A²-, A² represents a single bond and R^{11a} represents H, and Y represents —C(O)OR^{9b}, in which R^{9b} is other than H, reaction of a corresponding compound of formula I in which the C₁ alkylene group that T represents is unsubstituted with a C₁₋₆ alkyl formate in the presence of a suitable base;
(xl) for compounds of formula I in which X¹ represents -Q-X², Q represents a single bond and X² represents C₁₋₈ alkyl or heterocycloalkyl substituted α to the indole ring by a G¹ substituent in which G¹ represents -A¹-R^{11a}, A¹ represents
—OA⁵-, A⁵ represents a single bond and R^{11a} represents H, reaction of a corresponding compound of formula I in which X¹ represents H with a compound corresponding to a compound of formula VI, but in which X^{1b} represents -Q-X², Q represents a single bond and X² represents C₁₋₈ alkyl or heterocycloalkyl, both of which groups are substituted by a Z¹ group in which Z¹ represents ═O;
(xli) for compounds of formula I in which X¹ represents -Q-X², Q represents a single bond and X² represents C₂₋₈ alkyl substituted by a G¹ substituent in which G¹ represents -A¹-R^{11a}, A¹ represents —OA⁵-, A⁵ represents a single bond and R^{11a} represents H, reaction of a corresponding compound of formula I in which X² represents C₁₋₇ alkyl substituted by a Z¹ group in which Z¹ represents ═O, with the corresponding Grignard reagent derivative of a compound of formula V in which L² represents chloro, bromo or iodo, Q^{a} is a single bond and X² represents C₁₋₇ alkyl;
(xlii) for compounds of formula I in which X¹ represents -Q-X², Q represents a single bond, and X² represents C₁₋₈ alkyl or heterocycloalkyl, both of which are unsubstituted in the position α to the indole ring, reduction of a corresponding compound of formula I in which X² represents C₁₋₈ alkyl substituted a to the indole ring by a G¹ substituent in which G¹ represents -A¹-R^{11a}, A¹ represents
—OA⁵-, A⁵ represents a single bond and R^{11a} represents H, in the presence of a suitable reducing agent;
(xliii) for compounds of formula I in which X¹ represents -Q-X², Q represents a single bond and X² represents C₁₋₈ alkyl or heterocycloalkyl, neither of which are substituted by Z¹ in which Z¹ represents ═O, reduction of a corresponding compound of formula I in which X² represents C₁₋₈ alkyl or heterocycloalkyl, which groups are substituted by one or more Z¹ groups in which Z¹ represents ═O; or
(xliv) for compounds of formula I in which X¹ represents —N(R^{9a})-J-R^{10a}, reaction of a compound of formula XXIV as defined above, with a compound of formula VI in which X^{1b} represents —N(R^{9a})-J-R^{10a} and R^{9a}, R^{10a} and J are as defined in claim 1.
